(19) United States
(12) Patent Application Publication
(10) Pub. No.: US 2025/0092459 A1
(43) Pub. Date: Mar. 20, 2025

(54) NANOZYME-CATALYSED DETECTION OF NUCLEIC ACIDS

(71) Applicant: IMPERIAL COLLEGE INNOVATIONS LIMITED, London (GB)

(72) Inventors: Molly Morag STEVENS, LONDON (GB); Marta Broto AVILES, LONDON (GB); Christopher ADRIANUS, LONDON (GB); Schan DISSANAYAKE-PERERA, LONDON (GB)

(21) Appl. No.: 18/292,053

(22) PCT Filed: Jul. 26, 2022
(86) PCT No.: PCT/EP2022/070866

§ 371 Date: Jan. 25, 2024

(30) Foreign Application Priority Data
Jul. 26, 2021 (GB) 2110729.7
May 31, 2022 (GB) 2208018.8

(51) Int. Cl. C12Q 1/6883; C12N 9/22; C12N 15/11; C12Q 1/6816; G01N 33/543

(52) U.S. Cl. CPC C12Q 1/6883; C12N 9/22; C12N 15/11; C12Q 1/6816; G01N 33/54388; C12N 2310/20; C12N 2320/10; C12Q 2600/158

(57) ABSTRACT

The present disclosure relates to systems and methods of detecting nucleic acids using a combination of a Cas-based reaction with catalytic nanoparticles. Provided is a nucleic acid detection system for detecting one or more target nucleic acids, the system comprising a CRISPR effector protein; one or more guide RNAs (gRNA), each of which is specific for the one or more target nucleic acids; and a reporter RNA molecule; and a catalytic nanoparticle. Also provided is a reporter RNA molecule for a CRISPR detection assay. The disclosure also relates to uses, methods and kits for detecting one or more target nucleic acids. The uses, methods and kits extend to diagnosing a disease in a patient based on the presence of a nucleic acid of interest.

The present invention relates to systems and methods of detecting nucleic acids using a combination of a Cas-based reaction with a catalytic nanoparticle.

### BACKGROUND

CRISPR-based diagnostics have emerged as a useful tool that allows sensitive and specific detection of target DNA and RNA sequences. CRISPR-based diagnostics exploit Cas protein nuclease activity which can be triggered upon binding of a guide RNA (gRNA) to a complementary sequence. The nuclease activity can be measured through the subsequent cleavage of reporter molecules.

Due to the limited nuclease activity of Cas proteins, most current CRISPR-based diagnostics require the creation of a lot of copies of the target DNA or RNA, called preamplification. Preamplification allows such diagnostics to reach sufficient sensitivity for clinical applications. Nevertheless, this adds complexity to the reaction chemistry and limits quantification capability due to the exponential nature of most of the preamplification techniques.

Since 2017, CRISPR-based diagnostics have been used as a platform for the detection of nucleic acids [1,2,3,4]. In these assays, sensing of DNA or RNA is mediated through a complementary guide RNA (gRNA), which induces the activation of a Cas enzyme that indicates the presence of a target analyte. To this end, a wide variety of assays have been reported which differ in their usage of different Cas enzymes, reporter molecules or readout technologies.

To date, different advances in the design of assays that utilize Cas protein have focused in the usage of different Cas enzymes, reporter molecules or readout technologies. These different assay designs have allowed the sensitive and specific detection of different pathogens [5], a range of biomarkers of human disease [6,7], and genetic variants [2,8]. CRISPR-based diagnostics have also been applied to the sensing of small molecules or proteins [9].

However, to reach the clinically relevant concentrations of DNA or RNA in biological samples, most current CRISPR-based nucleic acid detection systems are combined with a preamplification step where many copies of the target RNA or DNA are produced. The main examples of this preamplification step are polymerase chain reaction (PCR) [10] or isothermal amplification methods [8]. While these primer-based amplification techniques allow some diagnostic assays to reach the concentration of single-molecule analysis, PCR requires thermal cycling which limits its use as a point-of-care diagnostic, and isothermal amplification requires controlled temperature, complex primer design [8,11], can suffer from non-specific amplification [12], and has restricted multiplexing capability [3].

Furthermore, preamplification strategies lack specificity for the detection of specific mutations or sensing of very short target sequences, such as microRNAs (miRs) since the introduction of these steps can restrict the flexibility to design an optimal gRNA. Thus, it is of utmost importance to develop a preamplification-free CRISPR-based diagnostic to fully exploit the usability and programmability of this technology and to enable the quantitative detection of nucleic acids with single-nucleotide specificity in resource limited settings, such as in point-of-case assays.

Some strategies have been employed to tackle some of these limitations, for example CRISPR type Ill RNA nucleases such as Csm6 [3] or Cas10 [13] have been used to achieve a 3.5-fold and 100-fold increase in sensitivity, respectively. Alternatively, the combination of two different gRNAs in the same reaction mixture has also shown to improve the sensitivity [14,15], although the degree of signal enhancement is limited and this approach is not suitable for very short targets such as miRs.

Here, we disclose the design of a new amplification-free assay named CrisprZyme. CrisprZyme is the combination of a Cas-based reaction with a Nanozyme-Linked ImmunoSorbent Assay (NLISA). NLISA quantifies the cleaved reporter RNA by catalysing the readout signal; it is a stepwise addition of reagents onto an immobilised surface that ends with the catalysis of a chromogenic substrate to generate a readout signal. The catalytic conversion rate of nanozymes, nanometer-sized catalytic metallic particles, is higher than their enzymatic counterparts [16], thus making NLISA even more sensitive than Enzyme-Linked ImmunoSorbent Assays (ELISA). Furthermore, NLISA offers high-throughput capability and ease of use.

We designed nanozymes that are immobilised through an RNA linker oligonucleotide and catalyse the oxidation of a chromogenic substrate. In the presence of the target RNA analyte, the reporter RNA gets cleaved by Cas13-mediated nuclease activity resulting in the absence of the step by step addition of reagents onto a solid support and resulting in the lack of colour change.

The present invention demonstrates that the combination of a CRISPR/Cas-based reaction with a Nanozyme-Linked ImmunoSorbent Assay (NLISA) (termed ‘CrisprZyme’), allows for the quantitative and colorimetric readout of Cas-mediated oligonucleotide detection through catalytic metallic nanoparticles (nanozymes). We validated CrisprZyme using synthetic standards and RNA isolated from cell lines, patient tissue biopsies and human plasma.

Biology and nanotechnology both inhabit the same length scale, with some biomolecules being on the order of 1-200 nm, which makes nanomaterials in this range ideally suited for use in biosensing applications. Nanoparticles have unique physicochemical properties and can be produced in a diverse range of well-controlled sizes, shapes, and can be capped with a variety of different ligands that enable stability in physiological environments. Based on the composition of the nanomaterial, a variety of functionalization chemistries (e.g. electrostatic, covalent, and physical adsorption) are available for modifying the surface with biomolecules, such as proteins, peptides, and nucleic acids that can be used for sensing.

Both inorganic catalysts and biological enzymes facilitate chemical reactions by lowering the activation energy, thus increasing the rate of reaction, allowing it to proceed at reduced temperatures and pressures. Biological enzymes are used in a variety of immunoassay configurations for amplification to enable sensitive analyte detection. However, enzymes suffer from instability in harsh environments such as varying pH and increasing temperature, and are susceptible to denaturation by proteases, hindering their application at the PoC.

This has motivated the development of artificial enzymes and nanomaterials with enzyme-like characteristics to replace their biological counterparts as labels in immunoassays. Over the last decade, a variety of nanomaterials have been explored to mimic the function and sometimes structure of biological enzymes. This class of materials is sometimes referred to as “nanozymes” or nanoparticles possessing enzyme-like characteristics, including peroxidase, oxidase, glucose oxidase, and catalase-like activities [17]. Of interest for immunoassay development is a class of nanomaterials with intrinsic peroxidase-like activity. Peroxidases are a class of metalloenzymes, containing an iron heme group, used to reduce hydrogen peroxide to water through a redox cycle. Peroxidases use hydrogen peroxide to oxidize both inorganic and organic compounds, such as TMB [18,19]. Under specific reaction conditions, iron oxide [20], carbon [21], gold [22], platinum [23], palladium [24], nickel [25] and iridium [26]nanoparticles have all demonstrated peroxidase-like activity through the oxidation of substrates in the presence of hydrogen peroxide.

Yan and coworkers [23] first reported the intrinsic peroxidase-like activity of ferromagnetic iron oxide nanoparticles, where Fe₃O₄ magnetic nanoparticles were used as labels to replace HRP in a simple solution-phase immunoassay. The ferromagnetic nanozymes showed high stability across a broad range of pH and temperature conditions and recyclability of the nanozyme label. Since this first “nanozyme” report over a decade ago, many new enzyme-mimetic nanoparticle formulations have been developed, spanning a broad range of morphologies, sizes, compositions and surface chemistries.

Since the catalytic activity of the nanomaterials is highly dependent on these physiochemical properties, Yan and coworkers [21] recently reported a standardized protocol to define nanozyme catalytic activity that will allow for comparison between different nanozyme formulations. The protocol involves calculating the catalytic efficiency of nanozymes by evaluating the Michaelis-Menten kinetics of peroxidase-mimicking nanomaterial catalyzed oxidation of chromogenic substrates to determine their catalytic constants.

CrisprZyme showed an improvement in sensitivity compared to the Cas13 reaction alone of 1000-fold, allowing the detection of a range of non-coding RNAs; namely microRNAs (miRNA), long non-coding RNAs (IncRNA) and circular RNAs (circRNA). We used CrisprZyme to classify cardiovascular disease patients with different levels of Inc-RNA correlated to the levels of high-sensitivity troponin T (hsTnT). We have also evaluated cellular differentiation in vitro by measuring the levels of a mi-RNA. This invention presents CripsrZyme as a universally applicable colorimetric readout catalyst for CRISPR-based diagnostics that provides preamplification-free and quantitative characteristics in highly sensitive diagnostics tests.

### SUMMARY OF THE INVENTION

This summary introduces concepts that are described in more detail in the detailed description. It should not be used to identify essential features of the claimed subject matter, nor to limit the scope of the claimed subject matter.

The present invention provides a nucleic acid detection system for detection of one or more target nucleic acids comprising:
a CRISPR detection composition comprising:
   a. a CRISPR effector protein;
   b. one or more guide RNAs (gRNA), each of which is specific for the one or more target nucleic acids; and
   c. a reporter RNA molecule; and
a catalytic nanoparticle.

In some embodiments, the nucleic acid detection system further comprises an RNase inhibitor.

In some embodiments, the CRISPR effector protein is Cas 13, Cas 12, Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3 or Csf4. In some embodiments, the CRISPR effector protein is Cas 13, for example Cas13a or Cas13b. In some embodiments, the CRISPR effector protein is Cas 12, for example Cas12a or Cas12b.

In some embodiments, the one or more target nucleic acids detected is RNA, optionally wherein the RNA is double stranded RNA or single stranded RNA. In some embodiments, the one or more target nucleic acids detected is DNA, optionally wherein the DNA is double stranded DNA or single stranded DNA.

In some embodiments, the nucleic acid detection system comprises more than one guide RNA, each of which is specific for a different target nucleic acid. In some embodiments, the nucleic acid detection system comprises 2, 3, 4, 5, 6, 7, 8, 9, 10, 15, 20, 25, 30, 35, 40, 45, 50, 55, 60, 65, 70, 75, 80, 85, 90, 95 or 100 or more guide RNAs, each of which is specific for a different target nucleic acid.

In some embodiments, the catalytic nanoparticle comprises one or more compounds selected from the list consisting of: platinum, iron oxide, carbon, gold, palladium, nickel and iridium. In some embodiments, the catalytic nanoparticle is a platinum, iron oxide, carbon, gold, palladium, nickel or iridium nanoparticle. In some embodiments, the catalytic nanoparticle comprises platinum and gold. In some embodiments, the catalytic nanoparticle is a platinum nanoparticle, for example a porous platinum nanoparticle.

In some embodiments, the average diameter of the catalytic nanoparticle is from about 1 nm to about 10 μm, for example about 1 nm to about 9 μm, about 1 nm to about 8 μm, about 1 nm to about 7 μm, about 1 nm to about 6 μm, about 1 nm to about 5 μm, about 1 nm to about 4 μm, about 1 nm to about 3 μm, about 1 nm to about 2 μm, about 1 nm to about 1 μm, about 1 nm to about 900 nm, about 1 nm to about 800 nm, about 1 nm to about 700 nm, about 1 nm to about 600 nm, about 1 nm to about 500 nm, about 1 nm to about 400 nm, about 1 nm to about 350 nm, about 1 nm to about 300 nm, about 1 nm to about 250 nm, about 1 nm to about 200 nm, about 1 nm to about 150 nm, about 1 nm to about 100 nm, about 1 nm to about 50 nm, about 1 nm to about 40 nm, about 1 nm to about 30 nm, about 1 nm to about 20 nm, about 1 nm to about 10 nm, about 10 nm to about 10 μm, about 10 nm to about 9 μm, about 10 nm to about 8 μm, about 10 nm to about 7 μm, about 10 nm to about 6 μm, about 10 nm to about 5 μm, about 10 nm to about 4 μm, about 10 nm to about 3 μm, about 10 nm to about 2 μm, about 10 nm to about 1 μm, about 10 nm to about 900 nm, about 10 nm to about 800 nm, about 10 nm to about 700 nm, about 10 nm to about 600 nm, about 10 nm to about 500 nm, about 10 nm to about 400 nm, about 10 nm to about 350 nm, about 10 nm to about 300 nm, about 10 nm to about 250 nm, about 10 nm to about 200 nm, about 10 nm to about 150 nm, about 10 nm to about 100 nm, about 10 nm to about 50 nm, about 10 nm to about 40 nm, about 10 nm to about 30 nm, about 10 nm to about 20 nm, about 20 nm to about 10 μm, about 20 nm to about 9 μm, about 20 nm to about 8 μm, about 20 nm to about 7 μm, about 20 nm to about 6 μm, about 20 nm to about 5 μm, about 20 nm to about 4 μm, about 20 nm to about 3 μm, about 20 nm to about 2 μm, about 20 nm to about 1 μm, about 20 nm to about 900 nm, about 20 nm to about 800 nm, about 20 nm to about 700 nm, about 20 nm to about 600 nm, about 20 nm to about 500 nm, about 20 nm to about 400 nm, about 20 nm to about 350 nm, about 20 nm to about 300 nm, about 20 nm to about 250 nm, about 20 nm to about 200 nm, about 20 nm to about 150 nm, about 20 nm to about 100 nm, about 20 nm to about 50 nm, about 20 nm to about 40 nm, about 20 nm to about 30 nm, about 30 nm to about 10 μm, about 30 nm to about 9 μm, about 30 nm to about 8 μm, about 30 nm to about 7 μm, about 30 nm to about 6 μm, about 30 nm to about 5 μm, about 30 nm to about 4 μm, about 30 nm to about 3 μm, about 30 nm to about 2 μm, about 30 nm to about 1 μm, about 30 nm to about 900 nm, about 30 nm to about 800 nm, about 30 nm to about 700 nm, about 30 nm to about 600 nm, about 30 nm to about 500 nm, about 30 nm to about 400 nm, about 30 nm to about 350 nm, about 30 nm to about 300 nm, about 30 nm to about 250 nm, about 30 nm to about 200 nm, about 30 nm to about 150 nm, about 30 nm to about 100 nm, about 30 nm to about 50 nm, about 30 nm to about 40 nm, about 40 nm to about 10 μm, about 40 nm to about 9 μm, about 40 nm to about 8 μm, about 40 nm to about 7 μm, about 40 nm to about 6 μm, about 40 nm to about 5 μm, about 40 nm to about 4 μm, about 40 nm to about 3 μm, about 40 nm to about 2 μm, about 40 nm to about 1 μm, about 40 nm to about 900 nm, about 40 nm to about 800 nm, about 40 nm to about 700 nm, about 40 nm to about 600 nm, about 40 nm to about 500 nm, about 40 nm to about 400 nm, about 40 nm to about 350 nm, about 40 nm to about 300 nm, about 40 nm to about 250 nm, about 40 nm to about 200 nm, about 40 nm to about 150 nm, about 40 nm to about 100 nm, about 40 nm to about 50 nm, about 50 nm to about 10 μm, about 50 nm to about 9 μm, about 50 nm to about 8 μm, about 50 nm to about 7 μm, about 50 nm to about 6 μm, about 50 nm to about 5 μm, about 50 nm to about 4 μm, about 50 nm to about 3 μm, about 50 nm to about 2 μm, about 50 nm to about 1 μm, about 50 nm to about 900 nm, about 50 nm to about 800 nm, about 50 nm to about 700 nm, about 50 nm to about 600 nm, about 50 nm to about 500 nm, about 50 nm to about 400 nm, about 50 nm to about 350 nm, about 50 nm to about 300 nm, about 50 nm to about 250 nm, about 50 nm to about 200 nm, about 50 nm to about 150 nm, about 50 nm to about 100 nm, about 100 nm to about 10 μm, about 100 nm to about 9 μm, about 100 nm to about 8 μm, about 100 nm to about 7 μm, about 100 nm to about 6 μm, about 100 nm to about 5 μm, about 100 nm to about 4 μm, about 100 nm to about 3 μm, about 100 nm to about 2 μm, about 100 nm to about 1 μm, about 100 nm to about 900 nm, about 100 nm to about 800 nm, about 100 nm to about 700 nm, about 100 nm to about 600 nm, about 100 nm to about 500 nm, about 100 nm to about 400 nm, about 100 nm to about 350 nm, about 100 nm to about 300 nm, about 100 nm to about 250 nm, about 100 nm to about 200 nm, about 100 nm to about 150 nm, about 150 nm to about 10 μm, about 150 nm to about 9 μm, about 150 nm to about 8 μm, about 150 nm to about 7 μm, about 150 nm to about 6 μm, about 150 nm to about 5 μm, about 150 nm to about 4 μm, about 150 nm to about 3 μm, about 150 nm to about 2 μm, about 150 nm to about 1 μm, about 150 nm to about 900 nm, about 150 nm to about 800 nm, about 150 nm to about 700 nm, about 150 nm to about 600 nm, about 150 nm to about 500 nm, about 150 nm to about 400 nm, about 150 nm to about 350 nm, about 150 nm to about 300 nm, about 150 nm to about 250 nm, about 150 nm to about 200 nm, about 200 nm to about 10 μm, about 200 nm to about 9 μm, about 200 nm to about 8 μm, about 200 nm to about 7 μm, about 200 nm to about 6 μm, about 200 nm to about 5 μm, about 200 nm to about 4 μm, about 200 nm to about 3 μm, about 200 nm to about 2 μm, about 200 nm to about 1 μm, about 200 nm to about 900 nm, about 200 nm to about 800 nm, about 200 nm to about 700 nm, about 200 nm to about 600 nm, about 200 nm to about 500 nm, about 200 nm to about 400 nm, about 200 nm to about 350 nm, about 200 nm to about 300 nm, about 200 nm to about 250 nm, about 250 nm to about 10 μm, about 250 nm to about 9 μm, about 250 nm to about 8 μm, about 250 nm to about 7 μm, about 250 nm to about 6 μm, about 250 nm to about 5 μm, about 250 nm to about 4 μm, about 250 nm to about 3 μm, about 250 nm to about 2 μm, about 250 nm to about 1 μm, about 250 nm to about 900 nm, about 250 nm to about 800 nm, about 250 nm to about 700 nm, about 250 nm to about 600 nm, about 250 nm to about 500 nm, about 250 nm to about 400 nm, about 250 nm to about 350 nm, about 250 nm to about 300 nm, about 300 nm to about 10 μm, about 300 nm to about 9 μm, about 300 nm to about 8 μm, about 300 nm to about 7 μm, about 300 nm to about 6 μm, about 300 nm to about 5 μm, about 300 nm to about 4 μm, about 300 nm to about 3 μm, about 300 nm to about 2 μm, about 300 nm to about 1 μm, about 300 nm to about 900 nm, about 300 nm to about 800 nm, about 300 nm to about 700 nm, about 300 nm to about 600 nm, about 300 nm to about 500 nm, about 300 nm to about 400 nm, about 300 nm to about 350 nm, about 350 nm to about 10 μm, about 350 nm to about 9 μm, about 350 nm to about 8 μm, about 350 nm to about 7 μm, about 350 nm to about 6 μm, about 350 nm to about 5 μm, about 350 nm to about 4 μm, about 350 nm to about 3 μm, about 350 nm to about 2 μm, about 350 nm to about 1 μm, about 350 nm to about 900 nm, about 350 nm to about 800 nm, about 350 nm to about 700 nm, about 350 nm to about 600 nm, about 350 nm to about 500 nm, about 350 nm to about 400 nm, about 400 nm to about 10 μm, about 400 nm to about 9 μm, about 400 nm to about 8 μm, about 400 nm to about 7 μm, about 400 nm to about 6 μm, about 400 nm to about 5 μm, about 400 nm to about 4 μm, about 400 nm to about 3 μm, about 400 nm to about 2 μm, about 400 nm to about 1 μm, about 400 nm to about 900 nm, about 400 nm to about 800 nm, about 400 nm to about 700 nm, about 400 nm to about 600 nm, about 400 nm to about 500 nm, about 500 nm to about 10 μm, about 500 nm to about 9 μm, about 500 nm to about 8 μm, about 500 nm to about 7 μm, about 500 nm to about 6 μm, about 500 nm to about 5 μm, about 500 nm to about 4 μm, about 500 nm to about 3 μm, about 500 nm to about 2 μm, about 500 nm to about 1 μm, about 500 nm to about 900 nm, about 500 nm to about 800 nm, about 500 nm to about 700 nm, about 500 nm to about 600 nm, about 600 nm to about 10 μm, about 600 nm to about 9 μm, about 600 nm to about 8 μm, about 600 nm to about 7 μm, about 600 nm to about 6 μm, about 600 nm to about 5 μm, about 600 nm to about 4 μm, about 600 nm to about 3 μm, about 600 nm to about 2 μm, about 600 nm to about 1 μm, about 600 nm to about 900 nm, about 600 nm to about 800 nm, about 600 nm to about 700 nm, about 700 nm to about 10 μm, about 700 nm to about 9 μm, about 700 nm to about 8 μm, about 700 nm to about 7 μm, about 700 nm to about 6 μm, about 700 nm to about 5 μm, about 700 nm to about 4 μm, about 700 nm to about 3 μm, about 700 nm to about 2 μm, about 700 nm to about 1 μm, about 700 nm to about 900 nm, about 700 nm to about 800 nm, about 800 nm to about 10 μm, about 800 nm to about 9 μm, about 800 nm to about 8 μm, about 800 nm to about 7 μm, about 800 nm to about 6 μm, about 800 nm to about 5 μm, about 800 nm to about 4 μm, about 800 nm to about 3 μm, about 800 nm to about 2 μm, about 800 nm to about 1 μm, about 800 nm to about 900 nm, about 900 nm to about 10 μm, about 900 nm to about 9 μm, about 900 nm to about 8 μm, about 900 nm to about 7 μm, about 900 nm to about 6 μm, about 900 nm to about 5 μm, about 900 nm to about 4 μm, about 900 nm to about 3 μm, about 900 nm to about 2 μm, about 900 nm to about 1 μm, about 1 μm to about 10 μm, about 1 μm to about 9 μm, about 1 μm to about 8 μm, about 1 μm to about 7 μm, about 1 μm to about 6 μm, about 1 μm to about 5 μm, about 1 μm to about 4 μm, about 1 μm to about 3 μm, about 1 μm to about 2 μm, about 2 μm to about 10 μm, about 2 μm to about 9 μm, about 2 μm to about 8 μm, about 2 μm to about 7 μm, about 2 μm to about 6 μm, about 2 μm to about 5 μm, about 2 μm to about 4 μm, about 2 μm to about 3 μm, about 3 μm to about 10 μm, about 3 μm to about 9 μm, about 3 μm to about 8 μm, about 3 μm to about 7 μm, about 3 μm to about 6 μm, about 3 μm to about 5 μm, about 3 μm to about 4 μm, about 4 μm to about 10 μm, about 4 μm to about 9 μm, about 4 μm to about 8 μm, about 4 μm to about 7 μm, about 4 μm to about 6 μm, about 4 μm to about 5 μm, about 5 μm to about 10 μm, about 5 μm to about 9 μm, about 5 μm to about 8 μm, about 5 μm to about 7 μm, about 5 μm to about 6 μm, about 6 μm to about 10 μm, about 6 μm to about 9 μm, about 6 μm to about 8 μm, about 6 μm to about 7 μm, about 7 μm to about 10 μm, about 7 μm to about 9 μm, about 7 μm to about 8 μm, about 8 μm to about 10 μm, about 8 μm to about 9 μm or about 9 μm to about 10 μm.

In some embodiments, the average diameter of the catalytic nanoparticle is about 100 nm to 300 nm, for example about 150 nm to about 250 nm. In some embodiments, the average diameter of the catalytic nanoparticle is about 188 nm.

In some embodiments, the catalytic nanoparticle is functionalised with high affinity binders such as biotin-binding proteins, antibodies, nanobodies, affibodies, peptides and/or aptamers. In some embodiments, the catalytic nanoparticle is functionalised with streptavidin, neutravidin, antiFAM or antidigoxigenin. In some embodiments, the catalytic nanoparticle is functionalised with streptavidin.

In some embodiments, the concentration of the CRISPR effector protein is from about 1 nM to about 500 nM, for example about 1 nM to about 450 nM, about 1 nM to about 400 nM, about 1 nM to about 350 nM, about 1 nM to about 300 nM, about 1 nM to about 250 nM, about 1 nM to about 200 nM, about 1 nM to about 150 nM, about 1 nM to about 100 nM, about 1 nM to about 90 nM, about 1 nM to about 80 nM, about 1 nM to about 70 nM, about 1 nM to about 60 nM, about 1 nM to about 50 nM, about 1 nM to about 40 nM, about 1 nM to about 30 nM, about 1 nM to about 20 nM, about 1 nM to about 10 nM, about 10 nM to about 500 nM, about 10 nM to about 450 nM, about 10 nM to about 400 nM, about 10 nM to about 350 nM, about 10 nM to about 300 nM, about 10 nM to about 250 nM, about 10 nM to about 200 nM, about 10 nM to about 150 nM, about 10 nM to about 100 nM, about 10 nM to about 90 nM, about 10 nM to about 80 nM, about 10 nM to about 70 nM, about 10 nM to about 60 nM, about 10 nM to about 50 nM, about 10 nM to about 40 nM, about 10 nM to about 30 nM, about 10 nM to about 20 nM, about 20 nM to about 500 nM, about 20 nM to about 450 nM, about 20 nM to about 400 nM, about 20 nM to about 350 nM, about 20 nM to about 300 nM, about 20 nM to about 250 nM, about 20 nM to about 200 nM, about 20 nM to about 150 nM, about 20 nM to about 100 nM, about 20 nM to about 90 nM, about 20 nM to about 80 nM, about 20 nM to about 70 nM, about 20 nM to about 60 nM, about 20 nM to about 50 nM, about 20 nM to about 40 nM, about 20 nM to about 30 nM, about 30 nM to about 500 nM, about 30 nM to about 450 nM, about 30 nM to about 400 nM, about 30 nM to about 350 nM, about 30 nM to about 300 nM, about 30 nM to about 250 nM, about 30 nM to about 200 nM, about 30 nM to about 150 nM, about 30 nM to about 100 nM, about 30 nM to about 90 nM, about 30 nM to about 80 nM, about 30 nM to about 70 nM, about 30 nM to about 60 nM, about 30 nM to about 50 nM, about 30 nM to about 40 nM, about 40 nM to about 500 nM, about 40 nM to about 450 nM, about 40 nM to about 400 nM, about 40 nM to about 350 nM, about 40 nM to about 300 nM, about 40 nM to about 250 nM, about 40 nM to about 200 nM, about 40 nM to about 150 nM, about 40 nM to about 100 nM, about 40 nM to about 90 nM, about 40 nM to about 80 nM, about 40 nM to about 70 nM, about 40 nM to about 60 nM, about 40 nM to about 50 nM, about 50 nM to about 500 nM, about 50 nM to about 450 nM, about 50 nM to about 400 nM, about 50 nM to about 350 nM, about 50 nM to about 300 nM, about 50 nM to about 250 nM, about 50 nM to about 200 nM, about 50 nM to about 150 nM, about 50 nM to about 100 nM, about 50 nM to about 90 nM, about 50 nM to about 80 nM, about 50 nM to about 70 nM, about 50 nM to about 60 nM, about 60 nM to about 500 nM, about 60 nM to about 450 nM, about 60 nM to about 400 nM, about 60 nM to about 350 nM, about 60 nM to about 300 nM, about 60 nM to about 250 nM, about 60 nM to about 200 nM, about 60 nM to about 150 nM, about 60 nM to about 100 nM, about 60 nM to about 90 nM, about 60 nM to about 80 nM, about 60 nM to about 70 nM, about 70 nM to about 500 nM, about 70 nM to about 450 nM, about 70 nM to about 400 nM, about 70 nM to about 350 nM, about 70 nM to about 300 nM, about 70 nM to about 250 nM, about 70 nM to about 200 nM, about 70 nM to about 150 nM, about 70 nM to about 100 nM, about 70 nM to about 90 nM, about 70 nM to about 80 nM, about 80 nM to about 500 nM, about 80 nM to about 450 nM, about 80 nM to about 400 nM, about 80 nM to about 350 nM, about 80 nM to about 300 nM, about 80 nM to about 250 nM, about 80 nM to about 200 nM, about 80 nM to about 150 nM, about 80 nM to about 100 nM, about 80 nM to about 90 nM, about 90 nM to about 500 nM, about 90 nM to about 450 nM, about 90 nM to about 400 nM, about 90 nM to about 350 nM, about 90 nM to about 300 nM, about 90 nM to about 250 nM, about 90 nM to about 200 nM, about 90 nM to about 150 nM, about 90 nM to about 100 nM, about 100 nM to about 500 nM, about 100 nM to about 450 nM, about 100 nM to about 400 nM, about 100 nM to about 350 nM, about 100 nM to about 300 nM, about 100 nM to about 250 nM, about 100 nM to about 200 nM, about 100 nM to about 150 nM, about 150 nM to about 500 nM, about 150 nM to about 450 nM, about 150 nM to about 400 nM, about 150 nM to about 350 nM, about 150 nM to about 300 nM, about 150 nM to about 250 nM, about 150 nM to about 200 nM, about 200 nM to about 500 nM, about 200 nM to about 450 nM, about 200 nM to about 400 nM, about 200 nM to about 350 nM, about 200 nM to about 300 nM, about 200 nM to about 250 nM, about 250 nM to about 500 nM, about 250 nM to about 450 nM, about 250 nM to about 400 nM, about 250 nM to about 350 nM, about 250 nM to about 300 nM, about 300 nM to about 500 nM, about 300 nM to about 450 nM, about 300 nM to about 400 nM, about 300 nM to about 350 nM, about 350 nM to about 500 nM, about 350 nM to about 450 nM, about 350 nM to about 400 nM, about 400 nM to about 500 nM, about 400 nM to about 450 nM or about 450 nM to about 500 nM.

In some embodiments, the concentration of the CRISPR effector protein is from about 100 nM to 500 nM, for example about 200 nM to about 400 nM. In some embodiments, the concentration of the CRISPR effector protein is about 300 nM.

In some embodiments, the CRISPR effector protein is derived from a bacterial strain selected from the list consisting of: Leptotrichia wadei (F0279), Leptotrichia shahii, Lachnospiraceae bacterium (MA2020), Lachnospiraceae bacterium (NK4A179), Clostridium aminophilum (DSM 10710), Carnobacterium gallinarum (DSM 4847), Paludibacter propionicigenes (WB4), Listeria weihenstephanensis (FSL R9-0317), Listeriaceae bacterium (FSL M6-0635), Listeria newyorkensis (FSL M6-0635), Rhodobacter capsulatus (SB 1003), Rhodobacter capsulatus (R121), Rhodobacter capsulatus (DE442), Leptotrichia wadei (Lw2), Leptotrichia buccalis (Lbu) and Listeria seeligeri. In some embodiments, the CRISPR effector protein is derived from Leptotrichia wadei.

In some embodiments, the CRISPR effector protein is a Cas13 protein, such as a Cas13a or Cas13b protein, derived from a bacterial strain selected from the list consisting of: Leptotrichia wadei (F0279), Leptotrichia shahii, Lachnospiraceae bacterium (MA2020), Lachnospiraceae bacterium (NK4A179), Clostridium aminophilum (DSM 10710), Carnobacterium gallinarum (DSM 4847), Paludibacter propionicigenes (WB4), Listeria weihenstephanensis (FSL R9-0317), Listeriaceae bacterium (FSL M6-0635), Listeria newyorkensis (FSL M6-0635), Rhodobacter capsulatus (SB 1003), Rhodobacter capsulatus (R121), Rhodobacter capsulatus (DE442), Leptotrichia wadei (Lw2), Leptotrichia buccalis (Lbu) and Listeria seeligeri. In some embodiments, the CRISPR effector protein is a Cas13 protein, such as a Cas13a or Cas13b protein, derived from Leptotrichia wadei.

In some embodiments, the CRISPR effector protein is a Cas12 protein, such as a Cas12a or Cas12b protein, derived from a bacterial strain selected from the list consisting of: Leptotrichia wadei (F0279), Leptotrichia shahii, Lachnospiraceae bacterium (MA2020), Lachnospiraceae bacterium (NK4A179), Clostridium aminophilum (DSM 10710), Carnobacterium gallinarum (DSM 4847), Paludibacter propionicigenes (WB4), Listeria weihenstephanensis (FSL R9-0317), Listeriaceae bacterium (FSL M6-0635), Listeria newyorkensis (FSL M6-0635), Rhodobacter capsulatus (SB 1003), Rhodobacter capsulatus (R121), Rhodobacter capsulatus (DE442), Leptotrichia wadei (Lw2), Leptotrichia buccalis (Lbu) and Listeria seeligeri. In some embodiments, the CRISPR effector protein is a Cas12 protein, such as a Cas12a or Cas12b protein, derived from Leptotrichia wadei.

In some embodiments, the concentration of the reporter RNA molecule is from about 0.1 nM to about 500 nM, for example about 0.1 nM to about 400 nM, about 0.1 nM to about 300 nM, about 0.1 nM to about 200 nM, about 0.1 nM to about 100 nM, about 0.1 nM to about 90 nM, about 0.1 nM to about 80 nM, about 0.1 nM to about 70 nM, about 0.1 nM to about 60 nM, about 0.1 nM to about 50 nM, about 0.1 nM to about 40 nM, about 0.1 nM to about 30 nM, about 0.1 nM to about 20 nM, about 0.1 nM to about 10 nM, about 0.1 nM to about 9 nM, about 0.1 nM to about 8 nM, about 0.1 nM to about 7 nM, about 0.1 nM to about 6 nM, about 0.1 nM to about 5 nM, about 0.1 nM to about 4 nM, about 0.1 nM to about 3 nM, about 0.1 nM to about 2 nM, about 0.1 nM to about 1 nM, about 0.1 nM to about 0.9 nM, about 0.1 nM to about 0.8 nM, about 0.1 nM to about 0.7 nM, about 0.1 nM to about 0.6 nM, about 0.1 nM to about 0.5 nM, about 0.1 nM to about 0.4 nM, about 0.1 nM to about 0.3 nM, about 0.1 nM to about 0.2 nM, about 0.2 nM to about 500 nM, about 0.2 nM to about 400 nM, about 0.2 nM to about 300 nM, about 0.2 nM to about 200 nM, about 0.2 nM to about 100 nM, about 0.2 nM to about 90 nM, about 0.2 nM to about 80 nM, about 0.2 nM to about 70 nM, about 0.2 nM to about 60 nM, about 0.2 nM to about 50 nM, about 0.2 nM to about 40 nM, about 0.2 nM to about 30 nM, about 0.2 nM to about 20 nM, about 0.2 nM to about 10 nM, about 0.2 nM to about 9 nM, about 0.2 nM to about 8 nM, about 0.2 nM to about 7 nM, about 0.2 nM to about 6 nM, about 0.2 nM to about 5 nM, about 0.2 nM to about 4 nM, about 0.2 nM to about 3 nM, about 0.2 nM to about 2 nM, about 0.2 nM to about 1 nM, about 0.2 nM to about 0.9 nM, about 0.2 nM to about 0.8 nM, about 0.2 nM to about 0.7 nM, about 0.2 nM to about 0.6 nM, about 0.2 nM to about 0.5 nM, about 0.2 nM to about 0.4 nM, about 0.2 nM to about 0.3 nM, about 0.3 nM to about 500 nM, about 0.3 nM to about 400 nM, about 0.3 nM to about 300 nM, about 0.3 nM to about 200 nM, about 0.3 nM to about 100 nM, about 0.3 nM to about 90 nM, about 0.3 nM to about 80 nM, about 0.3 nM to about 70 nM, about 0.3 nM to about 60 nM, about 0.3 nM to about 50 nM, about 0.3 nM to about 40 nM, about 0.3 nM to about 30 nM, about 0.3 nM to about 20 nM, about 0.3 nM to about 10 nM, about 0.3 nM to about 9 nM, about 0.3 nM to about 8 nM, about 0.3 nM to about 7 nM, about 0.3 nM to about 6 nM, about 0.3 nM to about 5 nM, about 0.3 nM to about 4 nM, about 0.3 nM to about 3 nM, about 0.3 nM to about 2 nM, about 0.3 nM to about 1 nM, about 0.3 nM to about 0.9 nM, about 0.3 nM to about 0.8 nM, about 0.3 nM to about 0.7 nM, about 0.3 nM to about 0.6 nM, about 0.3 nM to about 0.5 nM, about 0.3 nM to about 0.4 nM, about 0.4 nM to about 500 nM, about 0.4 nM to about 400 nM, about 0.4 nM to about 300 nM, about 0.4 nM to about 200 nM, about 0.4 nM to about 100 nM, about 0.4 nM to about 90 nM, about 0.4 nM to about 80 nM, about 0.4 nM to about 70 nM, about 0.4 nM to about 60 nM, about 0.4 nM to about 50 nM, about 0.4 nM to about 40 nM, about 0.4 nM to about 30 nM, about 0.4 nM to about 20 nM, about 0.4 nM to about 10 nM, about 0.4 nM to about 9 nM, about 0.4 nM to about 8 nM, about 0.4 nM to about 7 nM, about 0.4 nM to about 6 nM, about 0.4 nM to about 5 nM, about 0.4 nM to about 4 nM, about 0.4 nM to about 3 nM, about 0.4 nM to about 2 nM, about 0.4 nM to about 1 nM, about 0.4 nM to about 0.9 nM, about 0.4 nM to about 0.8 nM, about 0.4 nM to about 0.7 nM, about 0.4 nM to about 0.6 nM, about 0.4 nM to about 0.5 nM, about 0.5 nM to about 500 nM, about 0.5 nM to about 400 nM, about 0.5 nM to about 300 nM, about 0.5 nM to about 200 nM, about 0.5 nM to about 100 nM, about 0.5 nM to about 90 nM, about 0.5 nM to about 80 nM, about 0.5 nM to about 70 nM, about 0.5 nM to about 60 nM, about 0.5 nM to about 50 nM, about 0.5 nM to about 40 nM, about 0.5 nM to about 30 nM, about 0.5 nM to about 20 nM, about 0.5 nM to about 10 nM, about 0.5 nM to about 9 nM, about 0.5 nM to about 8 nM, about 0.5 nM to about 7 nM, about 0.5 nM to about 6 nM, about 0.5 nM to about 5 nM, about 0.5 nM to about 4 nM, about 0.5 nM to about 3 nM, about 0.5 nM to about 2 nM, about 0.5 nM to about 1 nM, about 0.5 nM to about 0.9 nM, about 0.5 nM to about 0.8 nM, about 0.5 nM to about 0.7 nM, about 0.5 nM to about 0.6 nM, about 0.6 nM to about 500 nM, about 0.6 nM to about 400 nM, about 0.6 nM to about 300 nM, about 0.6 nM to about 200 nM, about 0.6 nM to about 100 nM, about 0.6 nM to about 90 nM, about 0.6 nM to about 80 nM, about 0.6 nM to about 70 nM, about 0.6 nM to about 60 nM, about 0.6 nM to about 50 nM, about 0.6 nM to about 40 nM, about 0.6 nM to about 30 nM, about 0.6 nM to about 20 nM, about 0.6 nM to about 10 nM, about 0.6 nM to about 9 nM, about 0.6 nM to about 8 nM, about 0.6 nM to about 7 nM, about 0.6 nM to about 6 nM, about 0.6 nM to about 5 nM, about 0.6 nM to about 4 nM, about 0.6 nM to about 3 nM, about 0.6 nM to about 2 nM, about 0.6 nM to about 1 nM, about 0.6 nM to about 0.9 nM, about 0.6 nM to about 0.8 nM, about 0.6 nM to about 0.7 nM, about 0.7 nM to about 500 nM, about 0.7 nM to about 400 nM, about 0.7 nM to about 300 nM, about 0.7 nM to about 200 nM, about 0.7 nM to about 100 nM, about 0.7 nM to about 90 nM, about 0.7 nM to about 80 nM, about 0.7 nM to about 70 nM, about 0.7 nM to about 60 nM, about 0.7 nM to about 50 nM, about 0.7 nM to about 40 nM, about 0.7 nM to about 30 nM, about 0.7 nM to about 20 nM, about 0.7 nM to about 10 nM, about 0.7 nM to about 9 nM, about 0.7 nM to about 8 nM, about 0.7 nM to about 7 nM, about 0.7 nM to about 6 nM, about 0.7 nM to about 5 nM, about 0.7 nM to about 4 nM, about 0.7 nM to about 3 nM, about 0.7 nM to about 2 nM, about 0.7 nM to about 1 nM, about 0.7 nM to about 0.9 nM, about 0.7 nM to about 0.8 nM, about 0.8 nM to about 500 nM, about 0.8 nM to about 400 nM, about 0.8 nM to about 300 nM, about 0.8 nM to about 200 nM, about 0.8 nM to about 100 nM, about 0.8 nM to about 90 nM, about 0.8 nM to about 80 nM, about 0.8 nM to about 70 nM, about 0.8 nM to about 60 nM, about 0.8 nM to about 50 nM, about 0.8 nM to about 40 nM, about 0.8 nM to about 30 nM, about 0.8 nM to about 20 nM, about 0.8 nM to about 10 nM, about 0.8 nM to about 9 nM, about 0.8 nM to about 8 nM, about 0.8 nM to about 7 nM, about 0.8 nM to about 6 nM, about 0.8 nM to about 5 nM, about 0.8 nM to about 4 nM, about 0.8 nM to about 3 nM, about 0.8 nM to about 2 nM, about 0.8 nM to about 1 nM, about 0.8 nM to about 0.9 nM, about 0.9 nM to about 500 nM, about 0.9 nM to about 400 nM, about 0.9 nM to about 300 nM, about 0.9 nM to about 200 nM, about 0.9 nM to about 100 nM, about 0.9 nM to about 90 nM, about 0.9 nM to about 80 nM, about 0.9 nM to about 70 nM, about 0.9 nM to about 60 nM, about 0.9 nM to about 50 nM, about 0.9 nM to about 40 nM, about 0.9 nM to about 30 nM, about 0.9 nM to about 20 nM, about 0.9 nM to about 10 nM, about 0.9 nM to about 9 nM, about 0.9 nM to about 8 nM, about 0.9 nM to about 7 nM, about 0.9 nM to about 6 nM, about 0.9 nM to about 5 nM, about 0.9 nM to about 4 nM, about 0.9 nM to about 3 nM, about 0.9 nM to about 2 nM, about 0.9 nM to about 1 nM, about 1 nM to about 500 nM, about 1 nM to about 400 nM, about 1 nM to about 300 nM, about 1 nM to about 200 nM, about 1 nM to about 100 nM, about 1 nM to about 90 nM, about 1 nM to about 80 nM, about 1 nM to about 70 nM, about 1 nM to about 60 nM, about 1 nM to about 50 nM, about 1 nM to about 40 nM, about 1 nM to about 30 nM, about 1 nM to about 20 nM, about 1 nM to about 10 nM, about 1 nM to about 9 nM, about 1 nM to about 8 nM, about 1 nM to about 7 nM, about 1 nM to about 6 nM, about 1 nM to about 5 nM, about 1 nM to about 4 nM, about 1 nM to about 3 nM, about 1 nM to about 2 nM, about 2 nM to about 500 nM, about 2 nM to about 400 nM, about 2 nM to about 300 nM, about 2 nM to about 200 nM, about 2 nM to about 100 nM, about 2 nM to about 90 nM, about 2 nM to about 80 nM, about 2 nM to about 70 nM, about 2 nM to about 60 nM, about 2 nM to about 50 nM, about 2 nM to about 40 nM, about 2 nM to about 30 nM, about 2 nM to about 20 nM, about 2 nM to about 10 nM, about 2 nM to about 9 nM, about 2 nM to about 8 nM, about 2 nM to about 7 nM, about 2 nM to about 6 nM, about 2 nM to about 5 nM, about 2 nM to about 4 nM, about 2 nM to about 3 nM, about 3 nM to about 500 nM, about 3 nM to about 400 nM, about 3 nM to about 300 nM, about 3 nM to about 200 nM, about 3 nM to about 100 nM, about 3 nM to about 90 nM, about 3 nM to about 80 nM, about 3 nM to about 70 nM, about 3 nM to about 60 nM, about 3 nM to about 50 nM, about 3 nM to about 40 nM, about 3 nM to about 30 nM, about 3 nM to about 20 nM, about 3 nM to about 10 nM, about 3 nM to about 9 nM, about 3 nM to about 8 nM, about 3 nM to about 7 nM, about 3 nM to about 6 nM, about 3 nM to about 5 nM, about 3 nM to about 4 nM, about 4 nM to about 500 nM, about 4 nM to about 400 nM, about 4 nM to about 300 nM, about 4 nM to about 200 nM, about 4 nM to about 100 nM, about 4 nM to about 90 nM, about 4 nM to about 80 nM, about 4 nM to about 70 nM, about 4 nM to about 60 nM, about 4 nM to about 50 nM, about 4 nM to about 40 nM, about 4 nM to about 30 nM, about 4 nM to about 20 nM, about 4 nM to about 10 nM, about 4 nM to about 9 nM, about 4 nM to about 8 nM, about 4 nM to about 7 nM, about 4 nM to about 6 nM, about 4 nM to about 5 nM, about 5 nM to about 500 nM, about 5 nM to about 400 nM, about 5 nM to about 300 nM, about 5 nM to about 200 nM, about 5 nM to about 100 nM, about 5 nM to about 90 nM, about 5 nM to about 80 nM, about 5 nM to about 70 nM, about 5 nM to about 60 nM, about 5 nM to about 50 nM, about 5 nM to about 40 nM, about 5 nM to about 30 nM, about 5 nM to about 20 nM, about 5 nM to about 10 nM, about 5 nM to about 9 nM, about 5 nM to about 8 nM, about 5 nM to about 7 nM, about 5 nM to about 6 nM, about 6 nM to about 500 nM, about 6 nM to about 400 nM, about 6 nM to about 300 nM, about 6 nM to about 200 nM, about 6 nM to about 100 nM, about 6 nM to about 90 nM, about 6 nM to about 80 nM, about 6 nM to about 70 nM, about 6 nM to about 60 nM, about 6 nM to about 50 nM, about 6 nM to about 40 nM, about 6 nM to about 30 nM, about 6 nM to about 20 nM, about 6 nM to about 10 nM, about 6 nM to about 9 nM, about 6 nM to about 8 nM, about 6 nM to about 7 nM, about 7 nM to about 500 nM, about 7 nM to about 400 nM, about 7 nM to about 300 nM, about 7 nM to about 200 nM, about 7 nM to about 100 nM, about 7 nM to about 90 nM, about 7 nM to about 80 nM, about 7 nM to about 70 nM, about 7 nM to about 60 nM, about 7 nM to about 50 nM, about 7 nM to about 40 nM, about 7 nM to about 30 nM, about 7 nM to about 20 nM, about 7 nM to about 10 nM, about 7 nM to about 9 nM, about 7 nM to about 8 nM, about 8 nM to about 500 nM, about 8 nM to about 400 nM, about 8 nM to about 300 nM, about 8 nM to about 200 nM, about 8 nM to about 100 nM, about 8 nM to about 90 nM, about 8 nM to about 80 nM, about 8 nM to about 70 nM, about 8 nM to about 60 nM, about 8 nM to about 50 nM, about 8 nM to about 40 nM, about 8 nM to about 30 nM, about 8 nM to about 20 nM, about 8 nM to about 10 nM, about 8 nM to about 9 nM, about 9 nM to about 500 nM, about 9 nM to about 400 nM, about 9 nM to about 300 nM, about 9 nM to about 200 nM, about 9 nM to about 100 nM, about 9 nM to about 90 nM, about 9 nM to about 80 nM, about 9 nM to about 70 nM, about 9 nM to about 60 nM, about 9 nM to about 50 nM, about 9 nM to about 40 nM, about 9 nM to about 30 nM, about 9 nM to about 20 nM, about 9 nM to about 10 nM, about 10 nM to about 500 nM, about 10 nM to about 400 nM, about 10 nM to about 300 nM, about 10 nM to about 200 nM, about 10 nM to about 100 nM, about 10 nM to about 90 nM, about 10 nM to about 80 nM, about 10 nM to about 70 nM, about 10 nM to about 60 nM, about 10 nM to about 50 nM, about 10 nM to about 40 nM, about 10 nM to about 30 nM, about 10 nM to about 20 nM, about 20 nM to about 500 nM, about 20 nM to about 400 nM, about 20 nM to about 300 nM, about 20 nM to about 200 nM, about 20 nM to about 100 nM, about 20 nM to about 90 nM, about 20 nM to about 80 nM, about 20 nM to about 70 nM, about 20 nM to about 60 nM, about 20 nM to about 50 nM, about 20 nM to about 40 nM, about 20 nM to about 30 nM, about 30 nM to about 500 nM, about 30 nM to about 400 nM, about 30 nM to about 300 nM, about 30 nM to about 200 nM, about 30 nM to about 100 nM, about 30 nM to about 90 nM, about 30 nM to about 80 nM, about 30 nM to about 70 nM, about 30 nM to about 60 nM, about 30 nM to about 50 nM, about 30 nM to about 40 nM, about 40 nM to about 500 nM, about 40 nM to about 400 nM, about 40 nM to about 300 nM, about 40 nM to about 200 nM, about 40 nM to about 100 nM, about 40 nM to about 90 nM, about 40 nM to about 80 nM, about 40 nM to about 70 nM, about 40 nM to about 60 nM, about 40 nM to about 50 nM, about 50 nM to about 500 nM, about 50 nM to about 400 nM, about 50 nM to about 300 nM, about 50 nM to about 200 nM, about 50 nM to about 100 nM, about 50 nM to about 90 nM, about 50 nM to about 80 nM, about 50 nM to about 70 nM, about 50 nM to about 60 nM, about 60 nM to about 500 nM, about 60 nM to about 400 nM, about 60 nM to about 300 nM, about 60 nM to about 200 nM, about 60 nM to about 100 nM, about 60 nM to about 90 nM, about 60 nM to about 80 nM, about 60 nM to about 70 nM, about 70 nM to about 500 nM, about 70 nM to about 400 nM, about 70 nM to about 300 nM, about 70 nM to about 200 nM, about 70 nM to about 100 nM, about 70 nM to about 90 nM, about 70 nM to about 80 nM, about 80 nM to about 500 nM, about 80 nM to about 400 nM, about 80 nM to about 300 nM, about 80 nM to about 200 nM, about 80 nM to about 100 nM, about 80 nM to about 90 nM, about 90 nM to about 500 nM, about 90 nM to about 400 nM, about 90 nM to about 300 nM, about 90 nM to about 200 nM, about 90 nM to about 100 nM, about 100 nM to about 500 nM, about 100 nM to about 400 nM, about 100 nM to about 300 nM, about 100 nM to about 200 nM, about 200 nM to about 500 nM, about 200 nM to about 400 nM, about 200 nM to about 300 nM, about 300 nM to about 500 nM, about 300 nM to about 400 nM or about 400 nM to about 500 nM.

In some embodiments, the concentration of the reporter RNA is from about 0.1 nM to 2 nM, for example about 0.5 nM to about 1 nM. In some embodiments, the concentration of the reporter RNA is about 0.75 nM.

In some embodiments, the concentration of one or more of the components is the concentration of said component in the assay mastermix.

In some embodiments, the system is for use at a temperature of about 5° C. to about 50° C., for example about 5° C. to about 45° C., about 5° C. to about 40° C., about 5° C. to about 35° C., about 5° C. to about 30° C., about 5° C. to about 25° C., about 5° C. to about 20° C., about 5° C. to about 15° C., about 5° C. to about 10° C., about 10° C. to about 50° C., about 10° C. to about 45° C., about 10° C. to about 40° C., about 10° C. to about 35° C., about 10° C. to about 30° C., about 10° C. to about 25° C., about 10° C. to about 20° C., about 10° C. to about 15° C., about 15° C. to about 50° C., about 15° C. to about 45° C., about 15° C. to about 40° C., about 15° C. to about 35° C., about 15° C. to about 30° C., about 15° C. to about 25° C., about 15° C. to about 20° C., about 20° C. to about 50° C., about 20° C. to about 45° C., about 20° C. to about 40° C., about 20° C. to about 35° C., about 20° C. to about 30° C., about 20° C. to about 25° C., about 25° C. to about 50° C., about 25° C. to about 45° C., about 25° C. to about 40° C., about 25° C. to about 35° C., about 25° C. to about 30° C., about 30° C. to about 50° C., about 30° C. to about 45° C., about 30° C. to about 40° C., about 30° C. to about 35° C., about 35° C. to about 50° C., about 35° C. to about 45° C., about 35° C. to about 40° C., about 40° C. to about 50° C., about 40° C. to about 45° C. or about 45° C. to about 50° C. In some embodiments, the system is for use at a temperature of about 15° C. to about 37° C., for example about 20° C. or about 25° C.

In some embodiments, the system is for use in a multiwell plate format, glass slides, nitrocellulose or microfluidic device. In some embodiments, the system is for use in a 96-well or 384-well plate.

In some embodiments, the target nucleic acid is a messenger RNA (mRNA), non-coding RNA (ncRNA), microRNA (miRNA), a long non-coding RNA (IncRNA) or a circular RNA (circRNA). In some embodiments, the target nucleic acid is a DNA molecule, such as genomic DNA (gDNA) or complementary DNA (cDNA).

In some embodiments, the target nucleic acid is from a viral, bacterial or human source. In some embodiments, the target nucleic acid is from SARS-CoV-2. In some embodiments, the target nucleic acid is a cardiovascular event biomarker or a prostate cancer biomarker.

The present invention also provides a reporter RNA molecule for a CRISPR detection assay comprising at least two functional handles, for example two, three, four, five, six, seven, eight or more functional handles.

In some embodiments, the reporter RNA molecule comprises at least three functional handles, for example three, four, five, six, seven, eight or more functional handles. In some embodiments, the at least three functional handles comprise digoxigenin, biotin and FAM.

In some embodiments, the reporter RNA molecule comprises a first functional handle in the 5′-end. In some embodiments, the reporter RNA molecule comprises a second functional handle in the 3′-end, which is different to the first functional handle and has orthogonal binding to at the first functional handle of the 5′-end. In some embodiments, the reporter RNA molecule comprises a third functional handle with orthogonal binding to the first and second functional handles linked to either the 5′-end or 3′-end handle. In some embodiments, the third functional handle is attached to the reporter RNA via a covalent bond. In some embodiments, the covalent bond contains a spacer arm that can be any polymeric sequence comprising peptides, RNA or organic polymers. In some embodiments, the spacer arm comprises a PEG sequence of up to 10 KDa, for example a spacer arm comprising triethyleneglycol.

In some embodiments, the reporter RNA molecule comprises an RNA sequence of up to 50 nucleobases to be cleaved by an effector protein, such up to 10, 20, 30 or 40 nucleobases. In some embodiments, the reporter RNA molecule comprises an RNA sequence of 14 nucleobases to be cleaved by an effector protein. In some embodiments, the reporter RNA molecule comprises an RNA sequence of 6 nucleobases to be cleaved by an effector protein.

The present invention also provides a method of detecting a nucleic acid of interest comprising:
a. contacting a sample with a nucleic acid detection system for detection of one or more target nucleic acids comprising:
   i. a CRISPR detection composition comprising:
      1. a CRISPR effector protein;
      2. one or more guide RNAs (gRNA), each of which is specific for the one or more target nucleic acids; and
      3. a reporter RNA molecule; and
   ii. a catalytic nanoparticle;
b. adding a chromogenic substrate; and
c. detecting the presence of the nucleic acid of interest based on the catalysis of the chromogenic substrate.

The present invention also provides a method of detecting a nucleic acid of interest comprising:
a. contacting a sample with a nucleic acid detection system of the invention
b. adding a chromogenic substrate; and
c. detecting the presence of the nucleic acid of interest based on the catalysis of the chromogenic substrate.

In some embodiments, the presence of the nucleic acid of interest is used to diagnose a disease. In some embodiments, the presence of the nucleic acid of interest is used to diagnose a disease selected from the list consisting of respiratory diseases, HIV, tuberculosis, SARS-CoV-2, cardiovascular diseases, cancer or Alzheimer's disease.

The present invention also provides a method of diagnosing a disease in a patient comprising detecting a nucleic acid of interest in a biological sample derived from a patient comprising:
a. contacting a sample with a nucleic acid detection system for detection of one or more target nucleic acids comprising:
   i. a CRISPR detection composition comprising:
      1. a CRISPR effector protein;
      2. one or more guide RNAs (gRNA), each of which is specific for the one or more target nucleic acids; and
      3. a reporter RNA molecule; and
   ii. a catalytic nanoparticle;
b. adding a chromogenic substrate;
c. detecting the presence of the nucleic acid of interest based on the catalysis of the chromogenic substrate;
d. diagnosing the disease based on the presence of the nucleic acid of interest.

The present invention also provides a method of diagnosing a disease in a patient comprising detecting a nucleic acid of interest in a biological sample derived from a patient comprising:
a. contacting a sample with a nucleic acid detection system of the invention;
b. adding a chromogenic substrate; and
c. detecting the presence of the nucleic acid of interest based on the catalysis of the chromogenic substrate;
d. diagnosing the disease based on the presence of the nucleic acid of interest.

In some embodiments, the biological sample is a cell extract, a blood sample, for example a whole blood sample or a blood fraction such as blood serum, a tissue biopsy, amniotic fluid; aqueous humour; bile; blood plasma; breast milk; cerebrospinal fluid (CSF), endolymph, extracellular fluid, exudate, gastric acid, hemolymph, interstitial fluid, lymph, mucus, pericardial fluid, peritoneal fluid, perspiration (sweat), phlegm, pus, saliva, semen, synovial fluid, tears, urine, vaginal fluids, vomit, sputum, other biofluid or swab sample.

In some embodiments, the biological sample is a cell extract, a blood sample or a tissue biopsy. In some embodiments, detecting the presence of a nucleic acid of interest based on the catalysis of a chromogenic molecule comprises detecting the presence of a nucleic acid of interest based on generation of a chromogenic molecule. In some embodiments, detecting the presence of a nucleic acid of interest based on the catalysis of a chromogenic molecule comprises detecting the presence of a nucleic acid of interest based on the absence of a chromogenic molecule.

In some embodiments, the chromogenic substrate is selected from the list consisting of: TMB (3,3′,5,5′-tetramethylbenzidine), CN (4-chloro-1-naphthol) and DAB (3,3′-diaminobenzidine tetrahydrochloride), PNPP (p-Nitrophenyl Phosphate), ABTS (2,2′-Azinobis [3-ethylbenzothiazoline-6-sulfonic acid]), OPD (o-phenylenediamine dihydrochloride) and ONGP (ortho-Nitrophenyl-β-galactoside). In some embodiments, the chromogenic substrate is 3,3′,5,5′-tetramethylbenzidine (TMB).

In some embodiments, the concentration of the chromogenic substrate is from about 0.001% (w/v) to about 0.1% (w/v), for example about 0.001% (w/v) to about 0.09% (w/v), about 0.001% (w/v) to about 0.08% (w/v), about 0.001% (w/v) to about 0.07% (w/v), about 0.001% (w/v) to about 0.06% (w/v), about 0.001% (w/v) to about 0.05% (w/v), about 0.001% (w/v) to about 0.04% (w/v), about 0.001% (w/v) to about 0.03% (w/v), about 0.001% (w/v) to about 0.02% (w/v), about 0.001% (w/v) to about 0.01% (w/v), about 0.001% (w/v) to about 0.005% (w/v), about 0.001% (w/v) to about 0.002% (w/v), about 0.002% (w/v) to about 0.1% (w/v), about 0.002% (w/v) to about 0.09% (w/v), about 0.002% (w/v) to about 0.08% (w/v), about 0.002% (w/v) to about 0.07% (w/v), about 0.002% (w/v) to about 0.06% (w/v), about 0.002% (w/v) to about 0.05% (w/v), about 0.002% (w/v) to about 0.04% (w/v), about 0.002% (w/v) to about 0.03% (w/v), about 0.002% (w/v) to about 0.02% (w/v), about 0.002% (w/v) to about 0.01% (w/v), about 0.002% (w/v) to about 0.005% (w/v), about 0.005% (w/v) to about 0.1% (w/v), about 0.005% (w/v) to about 0.09% (w/v), about 0.005% (w/v) to about 0.08% (w/v), about 0.005% (w/v) to about 0.07% (w/v), about 0.005% (w/v) to about 0.06% (w/v), about 0.005% (w/v) to about 0.05% (w/v), about 0.005% (w/v) to about 0.04% (w/v), about 0.005% (w/v) to about 0.03% (w/v), about 0.005% (w/v) to about 0.02% (w/v), about 0.005% (w/v) to about 0.01% (w/v), about 0.01% (w/v) to about 0.1% (w/v), about 0.01% (w/v) to about 0.09% (w/v), about 0.01% (w/v) to about 0.08% (w/v), about 0.01% (w/v) to about 0.07% (w/v), about 0.01% (w/v) to about 0.06% (w/v), about 0.01% (w/v) to about 0.05% (w/v), about 0.01% (w/v) to about 0.04% (w/v), about 0.01% (w/v) to about 0.03% (w/v), about 0.01% (w/v) to about 0.02% (w/v), about 0.02% (w/v) to about 0.1% (w/v), about 0.02% (w/v) to about 0.09% (w/v), about 0.02% (w/v) to about 0.08% (w/v), about 0.02% (w/v) to about 0.07% (w/v), about 0.02% (w/v) to about 0.06% (w/v), about 0.02% (w/v) to about 0.05% (w/v), about 0.02% (w/v) to about 0.04% (w/v), about 0.02% (w/v) to about 0.03% (w/v), about 0.03% (w/v) to about 0.1% (w/v), about 0.03% (w/v) to about 0.09% (w/v), about 0.03% (w/v) to about 0.08% (w/v), about 0.03% (w/v) to about 0.07% (w/v), about 0.03% (w/v) to about 0.06% (w/v), about 0.03% (w/v) to about 0.05% (w/v), about 0.03% (w/v) to about 0.04% (w/v), about 0.04% (w/v) to about 0.1% (w/v), about 0.04% (w/v) to about 0.09% (w/v), about 0.04% (w/v) to about 0.08% (w/v), about 0.04% (w/v) to about 0.07% (w/v), about 0.04% (w/v) to about 0.06% (w/v), about 0.04% (w/v) to about 0.05% (w/v), about 0.05% (w/v) to about 0.1% (w/v), about 0.05% (w/v) to about 0.09% (w/v), about 0.05% (w/v) to about 0.08% (w/v), about 0.05% (w/v) to about 0.07% (w/v), about 0.05% (w/v) to about 0.06% (w/v), about 0.06% (w/v) to about 0.1% (w/v), about 0.06% (w/v) to about 0.09% (w/v), about 0.06% (w/v) to about 0.08% (w/v), about 0.06% (w/v) to about 0.07% (w/v), about 0.07% (w/v) to about 0.1% (w/v), about 0.07% (w/v) to about 0.09% (w/v), about 0.07% (w/v) to about 0.08% (w/v), about 0.08% (w/v) to about 0.1% (w/v), about 0.08% (w/v) to about 0.09% (w/v) or about 0.09% (w/v) to about 0.1% (w/v).

In some embodiments, the concentration of the chromogenic substrate is from about 0.005% (w/v) to about 0.02% (w/v). In some embodiments, the concentration of the chromogenic substrate is about 0.01% (w/v).

In some embodiments, the method further comprises addition of hydrogen peroxide (H₂O₂).

In some embodiments, the concentration of hydrogen peroxide is from about 0.001% (w/v) to about 0.1% (w/v), for example about 0.001% (w/v) to about 0.09% (w/v), about 0.001% (w/v) to about 0.08% (w/v), about 0.001% (w/v) to about 0.07% (w/v), about 0.001% (w/v) to about 0.06% (w/v), about 0.001% (w/v) to about 0.05% (w/v), about 0.001% (w/v) to about 0.04% (w/v), about 0.001% (w/v) to about 0.03% (w/v), about 0.001% (w/v) to about 0.02% (w/v), about 0.001% (w/v) to about 0.01% (w/v), about 0.001% (w/v) to about 0.005% (w/v), about 0.001% (w/v) to about 0.002% (w/v), about 0.002% (w/v) to about 0.1% (w/v), about 0.002% (w/v) to about 0.09% (w/v), about 0.002% (w/v) to about 0.08% (w/v), about 0.002% (w/v) to about 0.07% (w/v), about 0.002% (w/v) to about 0.06% (w/v), about 0.002% (w/v) to about 0.05% (w/v), about 0.002% (w/v) to about 0.04% (w/v), about 0.002% (w/v) to about 0.03% (w/v), about 0.002% (w/v) to about 0.02% (w/v), about 0.002% (w/v) to about 0.01% (w/v), about 0.002% (w/v) to about 0.005% (w/v), about 0.005% (w/v) to about 0.1% (w/v), about 0.005% (w/v) to about 0.09% (w/v), about 0.005% (w/v) to about 0.08% (w/v), about 0.005% (w/v) to about 0.07% (w/v), about 0.005% (w/v) to about 0.06% (w/v), about 0.005% (w/v) to about 0.05% (w/v), about 0.005% (w/v) to about 0.04% (w/v), about 0.005% (w/v) to about 0.03% (w/v), about 0.005% (w/v) to about 0.02% (w/v), about 0.005% (w/v) to about 0.01% (w/v), about 0.01% (w/v) to about 0.1% (w/v), about 0.01% (w/v) to about 0.09% (w/v), about 0.01% (w/v) to about 0.08% (w/v), about 0.01% (w/v) to about 0.07% (w/v), about 0.01% (w/v) to about 0.06% (w/v), about 0.01% (w/v) to about 0.05% (w/v), about 0.01% (w/v) to about 0.04% (w/v), about 0.01% (w/v) to about 0.03% (w/v), about 0.01% (w/v) to about 0.02% (w/v), about 0.02% (w/v) to about 0.1% (w/v), about 0.02% (w/v) to about 0.09% (w/v), about 0.02% (w/v) to about 0.08% (w/v), about 0.02% (w/v) to about 0.07% (w/v), about 0.02% (w/v) to about 0.06% (w/v), about 0.02% (w/v) to about 0.05% (w/v), about 0.02% (w/v) to about 0.04% (w/v), about 0.02% (w/v) to about 0.03% (w/v), about 0.03% (w/v) to about 0.1% (w/v), about 0.03% (w/v) to about 0.09% (w/v), about 0.03% (w/v) to about 0.08% (w/v), about 0.03% (w/v) to about 0.07% (w/v), about 0.03% (w/v) to about 0.06% (w/v), about 0.03% (w/v) to about 0.05% (w/v), about 0.03% (w/v) to about 0.04% (w/v), about 0.04% (w/v) to about 0.1% (w/v), about 0.04% (w/v) to about 0.09% (w/v), about 0.04% (w/v) to about 0.08% (w/v), about 0.04% (w/v) to about 0.07% (w/v), about 0.04% (w/v) to about 0.06% (w/v), about 0.04% (w/v) to about 0.05% (w/v), about 0.05% (w/v) to about 0.1% (w/v), about 0.05% (w/v) to about 0.09% (w/v), about 0.05% (w/v) to about 0.08% (w/v), about 0.05% (w/v) to about 0.07% (w/v), about 0.05% (w/v) to about 0.06% (w/v), about 0.06% (w/v) to about 0.1% (w/v), about 0.06% (w/v) to about 0.09% (w/v), about 0.06% (w/v) to about 0.08% (w/v), about 0.06% (w/v) to about 0.07% (w/v), about 0.07% (w/v) to about 0.1% (w/v), about 0.07% (w/v) to about 0.09% (w/v), about 0.07% (w/v) to about 0.08% (w/v), about 0.08% (w/v) to about 0.1% (w/v), about 0.08% (w/v) to about 0.09% (w/v) or about 0.09% (w/v) to about 0.1% (w/v).

In some embodiments, the concentration of the hydrogen peroxide is from about 0.01% (w/v) to about 0.03% (w/v). In some embodiments, the concentration of hydrogen peroxide is about 0.02% (w/v).

The present invention also provides use of a nucleic acid detection system of the invention in detection of a nucleic acid of interest. In some embodiments, the detection of a nucleic acid of interest is in a biological sample derived from a subject.

The present invention also provides a kit comprising:
i. a nucleic acid detection system according to the invention; and
ii. a point-of-care diagnostic test apparatus.

In some embodiments, the diagnostic test apparatus is a lateral flow immunoassay or a microfluidic chip. In some embodiments, the kit further comprises instructional material, optionally wherein the instructional material comprises instructions for conducting a method of detecting a nucleic acid of interest or diagnosing a disease in a patient according to the invention. In some embodiments, the kit further comprises a chromogenic substrate.

In some embodiments of the kits, the chromogenic substrate is selected from the list consisting of: TMB (3,3′,5,5′-tetramethylbenzidine), CN (4-chloro-1-naphthol) and DAB (3,3′-diaminobenzidine tetrahydrochloride), PNPP (p-Nitrophenyl Phosphate), ABTS (2,2′-Azinobis [3-ethylbenzothiazoline-6-sulfonic acid]), OPD (o-phenylenediamine dihydrochloride) and ONGP (ortho-Nitrophenyl-β-galactoside). In some embodiments, the chromogenic substrate is 3,3′,5,5′-tetramethylbenzidine (TMB).

In some embodiments of the kits, the diagnostic test apparatus is a lateral flow immunoassay. In some embodiments of the kits, the chromogenic substrate is 4-chloro-1-naphthol/3,3′-diaminobenzidine, tetrahydrochloride (CN/DAB).

The present invention also provides a diagnostic assay apparatus comprising the nucleic acid detection system of the invention.

The present invention describes an amplification-free CRISPR-based assay, CrisprZyme, which is able to accurately measure the concentration of a target RNA in complex samples by taking advantage of the high catalytic activity of metalling nanozymes (e.g. Pt@Au nanozymes). These nanozymes serve as signal enhancers of the NLISA used to quantify the cleavage of reporter RNA molecules. Upon functionalization of nanozymes with a biotin-binding protein, which was able to bind to one of the handles of the reporter RNA, we were able to measure reporter RNA concentration down to the femtomolar (fM) range.

CrisprZyme demonstrates an ability to accurately quantify synthetic RNA targets at room temperature and without any equipment with an LOD in the low pM range. This sensitivity represents up to about a 1000-fold improvement as compared to the Cas13a-based reaction alone. CrisprZyme design, specially the NLISA, is independent of the gRNA and Cas enzyme utilized in the assay, thus it can be used to amplify any CRISPR-based diagnostics which rely on the cleavage of reporter oligonucleotides.

The design of the assay described in this invention is composed of two parts: the Cas cleavage reaction and the NLISA (FIG. 1) to form the CrisprZyme assay when combined. CRISPR-based nucleic acid detection systems have been described in the literature, but the present invention combines the Cas reaction with a reporter RNA ultrasensitive-quantification NLISA.

NLISA is a technique that uses metallic catalytic particles (nanozymes) for the oxidation of a chromogenic substrate to produce a colorimetric signal. NLISA has been reported before in the literature for the analysis of proteins in solution in well-plate, but has never been used for the analysis of the cleavage of reporter RNA. Specifically, this NLISA takes advantage of highly catalytic platinum particles described in earlier work [27]. This previous work described the use of these particles in a lateral flow immunoassay for protein detection; instead, our NLISA exploits its catalytic activity for reporter RNA cleavage quantification.

CrisprZyme assay design is described in FIG. 1. In an exemplary embodiment using Cas13a, the sample containing the target RNA in solution is mixed with a mastermix which contains a buffer, RNase inhibitor, Cas13a protein, the RNA guide (gRNA) and a reporter RNA. When the target RNA is mixed with all the components of the mastermix, the target RNA interacts with its complementary gRNA and the Cas13a triggering a conformational change to Cas13a, which becomes a nuclease and cleaves the reporter RNA. The reporter RNA cleavage only happens in the presence of the target RNA in the sample. When the mastermix is mixed with the sample, it is known as Cas13a reaction. In some embodiments, the RNase inhibitor is added to prevent non-specific RNA degradation, for example due to contamination of the sample with RNase enzymes.

Cas13a is a CRISPR-associated (clustered regularly interspaced short palindromic repeats) endoribonuclease. This protein binds specifically to a gRNA sequence composed by two sections, the former is specific for the Cas protein [2] and the latter is specific for the desired target sequence and can be tailored to target different RNA sequences.

Upon binding of the Cas13a-gRNA with the target, the protein undergoes a conformational change and starts cleaving any RNA in solution with certain specificity, the cleavage mechanism is determined by the protein selected. In some embodiments a preferred Cas13a is produced in Leptotrichia wadei (LwaCas13a). The reporter RNA sequence (UUUUUC) has been described in the literature to be specific for LwaCas13a [3].

If using another Cas protein, either from another bacteria strain, or another type of Cas like Cas12, an alternative reporter oligonucleotide could be used and the invention could be used to detect DNA as the target molecule. This development could also be used to quantify DNA in an extracted sample if using Cas12a. In this case, the reporter oligonucleotide would have to be reporter DNA with a guide DNA specific for Cas12a. Targets could be single stranded DNA or double stranded DNA.

The nuclease activity of Cas can be quantified using a specific reporter oligonucleotide that has been designed with two binding handles in both 3′ and 5′ ends.

CrisprZyme provides a colorimetric readout that enables the determination of the absolute presence of a target oligonucleotide by the naked eye, or alternatively qualitative analysis through simple imaging, and quantitative analysis with a spectrometer. The different readout possibilities highlight the versatility of CrisprZyme in resource-limited settings, while its adaptability to 96 or 384-well plates enables high-throughput sample analysis at large scale. In addition, CrisprZyme followed by lateral-flow based readouts facilitate the rapid analysis of samples at the point of care, while the combination with LbuCas13a allows for the quantitative preamplification-free sensing in the femtomolar range.

Despite immunoassays being widely used to measure protein concentration, a plate-based analysis of the reporter RNA quantification in a sandwich complex format has never been described before. The incorporation of the plate format allows a high throughput capability and also the measurement of the results by simply taking a photo of the plate.

The present data (FIG. 4) shows that CrisprZyme can be used for the detection of different non-coding RNA species (namely miRs, IncRNAs and circRNAs). It is able to improve the sensitivity of CRISPR-based diagnostics and is able to quantitate oligonucleotides in complex samples without preamplification. We employed CrisprZyme to detect miRs indicative of cell differentiation in cultured cell extracts (FIG. 5), specifically from iPSCs to cardiomyocytes in cultured cell extracts. This presents CrisprZyme as a novel approach to monitor cell differentiation in research laboratories.

The present data (FIG. 5) also showed the use of CrisprZyme for the detection of Inc-LIPCAR in plasma from patients presenting with chest pain at hospital and circ-AURKA in tumor biopsies from patients with prostate cancer. Results correlated a significant difference between the patient with high and low levels of hsTnT and a clear performance capability for non-communicable diseases diagnosis. This also indicated the diagnostic potential of CrisprZyme in human biological samples.

CrisprZyme therefore offers the possibility for the preamplification-free, quantitative detection of ncRNA species in real samples. In an extension of the high-sensitivity of CrisprZyme, the technique could be combined with preamplification techniques to allow for the qualitative sensing of RNA at ultra-low concentrations.

The use of the NLISA detection method provides a number of advantages over method of the prior art, most notably a high-throughput assay that is field-deployable and requires no controlled temperature and no specialist preamplification equipment.

RNA/DNA has never been used before to form immunoassay bridge complexes bound to nanozymes. Furthermore, the present invention does not work with the concentration of reporter RNA described in prior art. Getting the two techniques to work together involves the step of a large decrease in reporter RNA concentration.

It has been described in other Cas-based biosensing the use of particles to produce a colorimetric readout by particle accumulation in a test strip. However, the signal produced by simple gold particle accumulation is limited to the optical density of the particles.

The present invention provides new particles with improved characteristics, specifically, platinum nanoparticles. These particles confer enzyme mimicking properties, with higher peroxidase activity than Horseradish peroxidase (HRP). The properties of these particles allow the enhancement of the generated signal by both optical density and chromophore oxidation upon substrate addition. The capability of these particles had never been explored before for molecular diagnostic applications, having previously been mostly used for protein detection.

We chose functionalization of the platinum nanoparticles with a biotin binding protein (streptavidin and neutravidin) since the immunoassay solid support is commercially available to adsorb antibodies, which fits with the binder to the other end of the reporter RNA, mAb-anti-FAM (fluorescein amidites, a single isomer derivative of fluorescein). This design could also be any other protein that binds to a small molecule with high affinity (e.g. anti-FAM, anti-FITC or anti-digoxigenin, antigen binding proteins, such as antibodies or antigen binding fragments thereof).

It is the first time these porous metallic nanoparticles composed of platinum and gold (Pt@Au) have been functionalised with streptavidin. The lack of a blocking step is an additional difference over methods of the prior art since most particles in the literature use some type of protein or polymer to block any surface unspecific interaction. In the assay of the present invention, and due to the characteristics of the reaction, this step is avoided showing better signal to noise ratio (FIG. 2).

Furthermore, the size was increased from 120 nm, of previously reported particles, to 188 nm (FIG. 3a,b) to provide the highest catalytic activity per binding event reporting better sensitivity for the detection of reporter RNA.

We optimised the amount of immunoreagents to run the NLISA, both the amount of capture antibody, the number of particles, and, finally, the quantity of reporter RNA we could detect (FIG. 15). This strategy was also applied both to 384- and 96-well plates obtaining comparable performance and proving the assay usability (FIG. 10).

The amount of reporter RNA added to the mastermix in this assay format (0.75 nM, FIG. 9) is approximately 500 times lower than in the literature [2] (375 nM). The reduction of the reporter RNA is a non-obvious step since it would be counter-intuitive to reduce the concentration of reporter RNA to improve the limit of detection (LOD). The reduction of the reporter RNA concentration allows CrisprZyme to reach a LOD in the pM concentration of target RNA.

Results showed that the cleavage buffer reported in the literature [2] was the better performing one and that Cas13a concentration was increased from 135 to 300 nM. We also tried both 37 and 22° C. to demonstrate that NLISA can also be done from 22 to 37° C. (FIG. 10).

The present invention also provides a new reporter RNA, composed of three functional handles (Digoxigenin-RNA-FAM-PEG-Biotin, wherein the functional handles are Digoxigenin, FAM and Biotin) which leads to improved sensitivity. This design was promoted by the need to increase the concentration of reporter RNA in solution. The only option to increase the reporter RNA concentration in solution, and consequently, improve the LOD would be to remove all the uncleaved reporter RNA. this new reporter provides inversed sensitivity, producing signal only in the event of target presence (FIG. 16), rather than target absence.

Removing uncleaved RNA from the solution can be achieved by this new reporter design. The cleavable RNA part is placed between Digoxigenin and FAM, the reporter molecule then becomes a PEG sequence with two handles (biotin and FAM). The inclusion of a non-cleavable part with PEG with two handles is the unique design of this reporter RNA.

In some embodiments the novel reporter RNA comprises a first functional handle in the 5′-end. In some embodiments the novel reporter RNA comprises a second functional handle in the 3′-end, which is different to the first functional handle and has orthogonal binding to at the first functional handle of the 5′-end. In some embodiments the first functional handle can be at the 3′-end and the second functional handle can be at the 5′-end.

In some embodiments the novel reporter RNA comprises an RNA sequence of up to 50 nucleobases to be cleaved by an effector protein, for example up to 10, 20, 30 or 40 nucleobases. In some embodiments the novel reporter RNA comprises an RNA sequence of up to 14 nucleobases to be cleaved by an effector protein. In some embodiments the novel reporter RNA comprises an RNA sequence of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleobases to be cleaved by an effector protein. In a preferred embodiment the novel reporter RNA comprises an RNA sequence of 6 nucleobases to be cleaved by an effector protein.

In some embodiments the reporter RNA comprises a third functional handle with orthogonal binding to the first and second functional handles linked to either the 5′-end or 3′-end handle. In some embodiments the third functional handle is attached to the reporter RNA via a covalent bond. In some embodiments the covalent bond could contain an extra spacer arm that can be any polymeric sequence comprising peptides, RNA or organic polymers. In some embodiments the spacer arm comprises a PEG sequence of up to 10 KDa. In a preferred embodiment, the spacer arm contains triethyleneglycol.

In a preferred embodiment the RNA reporter molecule has the following structure:

5′-/Digoxigenin/-/UUUUC/-/dT-Fam/-/Biotin-TEG/-3′

Wherein 5′-digoxigenin has the structure:

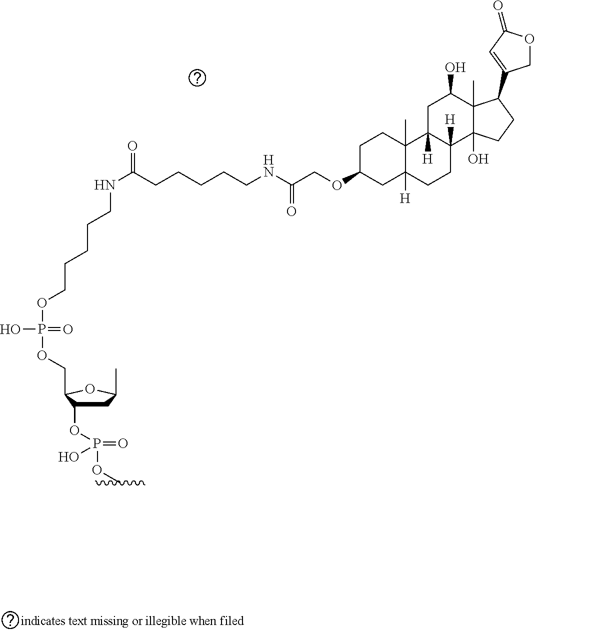

And Biotin-TEG has the structure:

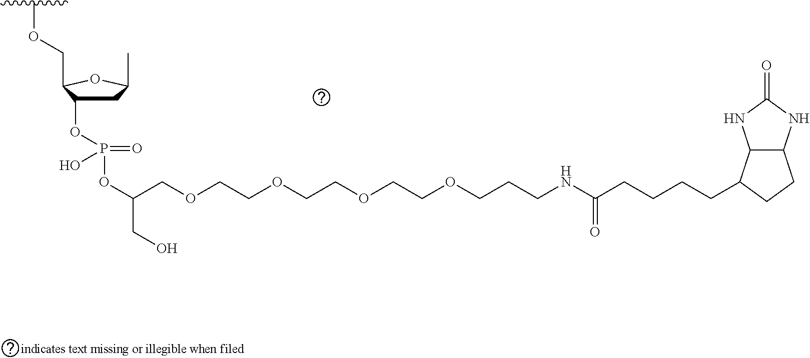

And dT-Fam, the internal modification is attached via an amino-dT building block.

In some embodiments, digoxigenin can be used as the 5′ functional handle, the 3′ functional handle or the internal modification.

In some embodiments, 6-fam can be used as the 5′ functional handle, the 3′ functional handle or the internal modification.

In some embodiments, fluorescein can be in the form of the two isomers 5-fam or 6-fam, or even the fluorescein derivative fluorescein isothiocyanate (FITC).

In some embodiments, biotin can comprise a triethyleneglycol (TEG) spacer in either the 5′ or 3′ end or in both the 5′ end and 3′ end.

In some embodiments TEG or heaxtheyleneglycol (HEX) can be introduced both at the 3′-end or 5′-end if a spacer is needed.

Briefly, the reporter (Digoxigenin-RNA-FAM-PEG-Biotin) is added to the Cas reaction. After cleavage of the RNA, it is mixed with immobilised antidigoxigenin, which will remove any reporter RNA reporter that contains digoxigenin and will leave the FAM-PEG-Biotin in solution. Then, the quantification of the signal is done through a sandwich complex between streptavidin and anti-FAM (FIG. 16). The handles of the reporter are interchangeable.

In some embodiments the functional handles of the RNA reporter molecules are any suitable binding sequences or molecules which bind to a binding partner. Suitable binding sequences or molecules are well known in the art (e.g., Digoxigenin, FAM, Biotin, HA tag, Myc tag, Flag tag, His tag). Any binding sequence which has a suitable binding partner (e.g. an antibody or antigen binding fragment thereof) is suitable for use as the functional handle of an RNA reporter molecule.

In some embodiments the nucleic acid detection system can be used in resource-limited settings. In some embodiments the nucleic acid detection system can be used in research (such as academic, government institute, pharmaceutical and biotechnology companies). In some embodiments the nucleic acid detection system can be used in a medical or healthcare setting, such as clinics (localised and delocalised health-centres, primary care, resource limited settings or localised and delocalised diagnostic laboratories).

Oligonucleotide analysis needs are related to clinical diagnosis and cell biology characterization. In some embodiments the nucleic acid detection system can be used for one or more of the following diagnostic purposes:
a. Gene mutation identification, such as to identify someone's increased risk of a disease, the presence of a genetic disease and monitor patient treatment.
b. Identification of different viral strains, such as to monitor outbreaks and track patient treatment
c. Determine the presence of a virus, such as to diagnose a patient with a viral infection. In some embodiments the diagnostics assay can be used to diagnose a patient with a SARS-CoV-2 infection.
d. Detection of the presence of a certain oligonucleotide sequence, such as to diagnose non-communicable diseases via the detection of disease biomarkers.
e. Quantification of up regulation and downregulation of mRNA and non-coding RNA, such as to diagnose a disease, or to monitor changes in a cell culture or tissue treatment.

In specific embodiments, the nucleic acid detection system of the invention can be used to analyse a viral outbreak in the field, that would like to determine the viral strain immediately. The qualitative results could be read with a simple device such as a smartphone.

In specific embodiments, the nucleic acid detection system of the invention can be used to test some materials with cells and to identify changes in mRNA regulation without needing to use complex, temperature controlled amplification methods such as PCR.

In specific embodiments, the nucleic acid detection system of the invention can be used to correlate the findings of a new protein biomarker with a gene mutation.

In specific embodiments, the nucleic acid detection system of the invention can be used determine a viral strain in a biological sample or patient without having to ship the samples to a centralised diagnostic laboratory.

In specific embodiments, the nucleic acid detection system of the invention can be used to diagnose one or more diseases in a patient. In specific embodiments, the nucleic acid detection system of the invention can be used to diagnose infectious diseases like HIV, tuberculosis, SARS-CoV-2. In specific embodiments, the nucleic acid detection system of the invention can be used to diagnose respiratory infections. In specific embodiments, the nucleic acid detection system of the invention can be used to diagnose non-communicable diseases like cardiovascular diseases, cancer or Alzheimer's disease.

In specific embodiments, the nucleic acid detection system of the invention can be used to diagnose SARS-CoV-2 presence in a swab sample, such as a nasopharyngeal swab sample. In specific embodiments, the nucleic acid detection system of the invention can be used to diagnose IncLIPCAR in patients at risk of heart failure. In specific embodiments, the nucleic acid detection system of the invention can be used to determine miR150-5p concentration as a predictive biomarker for preterm birth. In specific embodiments, the nucleic acid detection system of the invention can be used to determine miR141 levels in human serum for diagnosis of human prostate cancer. In specific embodiments, the nucleic acid detection system of the invention can be used to determine miR143-3p levels in cardiomyocyte cells differentiated from pluripotent stem cells (iPSC).

### BRIEF DESCRIPTION OF THE DRAWINGS

Embodiments of the present invention will now be described, by way of example only, with reference to the accompanying drawings, in which:

FIG. 1—CrisprZyme assay scheme. Schematic of the combination of a Cas-based reaction with a Nanozyme Linked Immunosorbent Assay (NLISA) proposed in this study. Target RNA is mixed with the gRNA-Cas13 complex and reporter RNA to develop the CRISPR reaction. Subsequently, the mixture is added to an immunoassay plate precoated with anti-6-carboxyfluorescein antibody (anti-FAM). The unbound reporter RNA is washed, and the nanozymes are added to form a complex through the bound reporter RNA. Finally, the substrate is added for colour development.

FIG. 2—Pt@Au functionalised with streptavidin show the best NLISA performance. a. Characterisation of the functionalised Pt@Au by S/N ratio and b. dynamic light scattering (DLS) with PBST and PBST supplemented with two different blocking agents: beta-casein and bovine serum albumin (BSA). c. Structural characterisation of non-functionalised and streptavidin-functionalised Pt@Au using transmission electron microscopy (TEM) and brightfield scanning-transmission electron microscopy (BF-STEM). TEM and BF-STEM images show that functionalised particles exhibited a thin layer of amorphous substances on the surface, presumably due to the presence of the functionalised streptavidin (arrows). d-e, STEM-Energy Dispersive X-ray Spectroscopy (EDS) analysis of streptavidin-functionalised Pt@Au. d, Representative high-angle annular dark-field (HAADF)-STEM image and EDS elemental mapping (Pt and Au). A merged image of Pt and Au maps is shown. e, Representative EDS spectra recorded from the whole area of the individual particle. Control corresponds to non-functionalised particles. Data represent individual measurements. Error bars represent SD (n>3 measurements). A₄₅₀: Absorbance measure at 450 nm.

FIG. 3—CrisprZyme detects synthetic RNA down to pM concentration. a, Number distribution of the hydrodynamic diameter of the Pt@Au prepared by overgrowing different amounts of Pt onto the surface of AuNP seeds or 120 nm Pt@Au. Data represent the mean+/−SD (n=3 measurements). b, Sigmoidal regression curve of the reporter RNA with streptavidin-Pt@Au of different sizes. Data represent the mean+/−SD (n=2 replicates). A₄₅₀: Absorbance measure at 450 nm. c, Photograph of CrisprZyme results in a 384-well plate; two replicates were performed for each concentration. Low concentration of target RNA to the left; high concentration to the right (5-fold dilutions of synthetic RNA in duplicate from right to left—2000 pM, 400 pM, 80 pM, 16 pM, 3.2 pM and 0 pM). d, Sigmoidal regression curve parameters. The data have been extracted from the four-parameter equation (Eq. 1) used to fit the standard curve. e, Sigmoidal regression of the CrisprZyme of a synthetic RNA target. Data were obtained measuring the absorbance of each well with a plate reader at 450 nm (A₄₅₀). f, Sigmoidal regression of the CrisprZyme of a synthetic RNA target. Data were obtained taking a photo of the results and recording the blue intensity of the central pixel. e,f, Data represent the mean+/−SD (n=2 replicates).

FIG. 4—CrisprZyme expands the dynamic range of Cas13-based diagnostics enabling the quantitative sensing of different non-coding RNA species. a, Comparison of the dynamic range of Cas13-based reaction and CrisprZyme. IC10 and IC90 values have been used to define the dynamic range of each assay. The data have been extracted from the four-parameter equation (FIG. 11) used to fit the standard curves shown in FIGS. 4b and 4c. Lighter shade indicates the concentration range for which quantitative measurements can be obtained. b, Standard curve for the detection of serial dilutions of synthetic RNA with Cas13-based reaction only. RFU 490ex/520em: Relative fluorescent units after excitation at 490 nm wavelength and measurements of emitted light at 520 nm. c, Standard curves for the detection of serial dilutions of synthetic RNA with CrisprZyme. b,c, Data and error bars represent mean±±SD (N≥2 experiments, n=2 replicates). d, Comparison of the performance of CrisprZyme with different ncRNA targets, Inc-LIPCAR: long non-coding RNA LIPCAR, miR-223: microRNA 223, Control: Synthetic RNA control, circ-AURKA: circular AURKA RNA. Data represent individual measurements. Error bars represent SD (n=2 replicates). e, Detection of miR-223 with CrisprZyme or rt-qPCR in 5 different samples at the indicated concentrations. Ct: Cycle threshold for PCR reactions. Data represent individual measurements. Error bars represent SD (n≥2 replicates). c,d,e, A₄₅₀ Absorbance measure at 450 nm.

FIG. 5—Quantification of different ncRNA species from cell culture and human plasma or tissue. a, Schematic representation of the sample analysis experimental workflow. b, Schematic of upregulated miRs upon differentiation of induced pluripotent stem cells (iPSCs) to cardiomyocytes (CMs) in vitro (left). Expression of miR-143-3p in iPSCs and CMs as measured by CrisprZyme. (N=4 samples, n=2 replicates) c, Schematic of upregulated IncRNAs in plasma of patients with heart failure. Expression of Inc-LIPCAR in patients with heart failure and a control group as measured by CrisprZyme. hsTNT: high sensitivity Troponin T. Data represent mean (N≥25 samples, n=2 replicates). d, Schematic of upregulated (black) circRNAs (grey) in tissue biopsies from patients with prostate cancer. Expression of circular AURKA RNA (circ-AURKA) in biopsies of prostate adenocarcinoma (ACA) and neuroendocrine prostate carcinoma (NEPC) as measured by CrisprZyme. Data represent mean (N=10 samples, n=2 replicates) (b,c,d). A₄₅₀: Absorbance measure at 450 nm. Box and whiskers plots represent median and quartiles. Mann Whittney test.

FIG. 6—Signal to noise (S/N) ratio achieved in the NLISA format with differently functionalized Pt@Au (streptavidin or neutravidin). a,b, Effect of different pH of buffers for the electrostatic interaction. a, Streptavidin functionalized particles were added at 0.5 pM b, Neutravidin functionalized particles were added at 1.25 pM c,d, Different amounts of added protein for functionalization. a,b,c,d, Data and error bars represent mean±SD (n≥1 replicates). A₄₅₀: Absorbance measure at 450 nm.

FIG. 7—Representative TEM images of streptavidin-functionalized Pt@Au. Scale bars: length of the bar as indicated below the bar.

FIG. 8—Structural and elemental analysis of Pt@Au. a, Representative HAADF-STEM images and EDS elemental mapping (Pt, Au, O, C, N) of Pt@Au. Merged images of Pt and Au maps are shown. b, Intensity line profile across the HAADF image. c. EDS point spectra obtained from different regions on Pt@Au (periphery (point 1; left) and core (point 2; right)) that correspond to the x-marked positions on each image.

FIG. 9—Effect of different components on the performance of CrisprZyme. a, Effect of buffer salts in the mastermix. Cleavage buffer (20 mM HEPES, 60 mM NaCl, 9 mM MgCl₂, pH 6), nuclease buffer (40 mM Tris-HCl, 60 mM NaCl, 6 mM MgCl₂, pH 7.3), NEB2 buffer (10 mM Tris-HCl, 50 mM NaCl, 10 mM MgCl₂, 1 mM DTT, pH 7.9) b, Effect of Cas13a concentration in the mastermix. c. Effect of the molar equivalents of gRNA:Cas13. d, Effect of the reporter RNA concentration in the mastermix. e. Effect of the presence of background RNA in spiked samples solution, polyA. a,b,c,d,e, Each experiment was conducted sequentially keeping the rest of parameters of the reaction constant. Data and error bars represent mean±SD (n=2 replicates). Results were normalized defining 0% as the smallest mean in each data set and 100% as the largest mean in each dataset. A₄₅₀: Absorbance measure at 450 nm.

FIG. 10—CrisprZyme simplicity evaluation. a, CrisprZyme at different temperatures. Data and error bars represent mean±SD (n=2 replicates). b, CrisprZyme in different plate sizes. Data and error bars represent mean±SD (n≥≥1 replicates). a,b, A₄₅₀: Absorbance measure at 450 nm.

FIG. 11—Sigmoidal regression curve parameters. Values represent mean±SD (N=2 experiments, n=2 replicates). LOD: limit of detection.

FIG. 12—Synthetic Targets

FIG. 13—Primers

FIG. 14—gRNA constructs

FIG. 15—A. 2D assay varying the concentration of AntiFAM and different amounts of particles. AntiFAM was coated at a concentration of 100 ng/mL. B. Calibration curve of different reporters. These reporters showed a LOD in the fM range of the NLISA.

FIG. 16—Proposed mechanism using new reporter RNA. Top drawing showing the assay in the presence of target, bottom drawing showing the assay in the absence of target. Briefly, in the presence of target RNA, the reporter RNA with three functional handles is cleaved by the Cas protein leaving in solution a part of the reporter RNA with only two functional handles. The cleaved reporter RNA is not captured by the first set of capture antibody (anti-digoxigenin) and is able to form the complex in the last step of the assay between streptavidin and anti-FITC PtNC (platinum nanocatalysts). This assay design provides increased colorimetric signal at higher concentrations of target RNA.

FIG. 17—Pt@Au functionalised with streptavidin shows the best NLISA performance. Sigmoidal regression curve of the reporter RNA with streptavidin-Pt@Au of different sizes. Data represent the mean+/−SD (n=2 replicates). A450: Absorbance measure at 450 nm.

FIG. 18—CrisprZyme detects synthetic RNA down to pM concentration. a, Photograph of CrisprZyme results in a 384-well plate; six replicates were performed for each concentration. Low concentration of target RNA to the left; high concentration to the right. b, Sigmoidal regression of the CrisprZyme of a IncRNA target. Data were obtained measuring the absorbance of each well with a plate reader at 450 nm (A450). c, Sigmoidal regression of the CrisprZyme of a IncRNA target. Data were obtained taking a photo of the results and recording the blue intensity of each well set as a region of interest. b,c, Data represent the mean+/−SD (n=6 replicates). d, Schematic of the combination of a Cas-based reaction with a nanozyme-amplified lateral flow assay proposed in this study. Target RNA is mixed with the gRNA-Cas13 complex and reporter RNA to develop the CRISPR reaction. Subsequently, streptavidin-functionalised nanozymes were mixed with CRISPR reaction product containing the biotinylated reporter RNA to form a complex. A test strip preprinted with anti-6-carboxyfluorescein antibody (anti-FAM) was used to draw the mixture. The uncleaved reporter RNA-nanozymes complexes were captured at the test line. Finally, the substrate is added for colour development. e, Detection of a serial dilution of Inc-LIPCAR with nanozyme-amplified lateral flow assay. Photographs show the test bands of the lateral flow test strips after completion of the assay without (top) and with (bottom) the substrate added for enhanced signal. f, Sigmoidal regression of the Inc-LIPCAR target for the nanozyme-amplified lateral flow assay. Data were obtained by normalising the pixel density of the test line relative to the internal control line of the test strip. g, Sigmoidal regression curve parameters. The data have been extracted from the four-parameter equation (Eq. 2) used to fit the standard curve.

FIG. 19—CrisprZyme expands the dynamic range of Cas13-based diagnostics enabling the quantitative sensing of different non-coding RNA species. a, Standard curves for the detection of serial dilutions of a IncRNA with CrisprZyme or combination of rt-RPA and CrisprZyme. Data represent the mean+/−SD (n=6 replicates). b, Comparison of the performance of CrisprZyme with different ncRNA targets, Inc-LIPCAR: long non-coding RNA LIPCAR, miR-223: microRNA 223, ncRNA: synthetic RNA, circ-AURKA: circular AURKA RNA. Data represent the mean+/−SD (n≥3 replicates).

FIG. 20—2D assay comparing anti-FAM and streptavidin-Pt@Au concentrations. Data show single measurements. A450: Absorbance measure at 450 nm.

FIG. 21—CrisprZyme simplicity evaluation. CrisprZyme with other non-targeting RNA from HEK cells. Data and error bars represent mean±SD (n=6 replicates). a,b Results were normalized defining 0% as the smallest mean in each data set and 100% as the largest mean in each dataset. A450: Absorbance measure at 450 nm.

FIG. 22—Cost comparison of different molecular diagnostic technologies and CrisprZyme. Information and prices extracted from suppliers' websites. Sherlock and CrisprZyme price accounts for reagent price and not commercialised assay prices. CrisprZyme assay price is GBP190/364 reactions.

FIG. 23—LIPCAR detection based on fluorescence readout. Sigmoidal regression of the Cas13a reaction using LwaCas13a for Inc-LIPCAR target RNA detection. Data were obtained measuring the fluorescence of each well with a plate reader at room temperature (λex=490 nm λem=520 nm). Data and error bars represent mean±SD (n=3 replicates).

FIG. 24—CrisprZyme with LbuCas13a. LbuCas13a reaction was done at 37° C. Data and error bars represent mean±SD (n=3 replicates).

FIG. 25—Time comparison of time to run the proposed assays.

FIG. 26—Sigmoidal regression curve parameters. Values represent mean±SD (n=3-6 replicates). LOD: limit of detection.

### DETAILED DESCRIPTION OF THE INVENTION

In order for the present invention to be more readily understood, certain terms are first defined below. Additional definitions for the following terms and other terms are set forth throughout the specification.

Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%), 6%), 5%, 4%, 3%, 2%, 1%), or less in either direction (greater than or less than) of the stated reference value unless otherwise stated or otherwise evident from the context (except where such number would exceed 100% of a possible value).

The term “comparable”, as used herein, refers to a system, set of conditions, effects, or results that is/are sufficiently similar to a test system, set of conditions, effects, or results, to permit scientifically legitimate comparison. Those of ordinary skill in the art will appreciate and understand which systems, sets of conditions, effects, or results are sufficiently similar to be “comparable” to any particular test system, set of conditions, effects, or results as described herein.

The term “correlates”, as used herein, has its ordinary meaning of “showing a correlation with”. Those of ordinary skill in the art will appreciate that two features, items or values show a correlation with one another if they show a tendency to appear and/or to vary, together. In some embodiments, a correlation is statistically significant when its p-value is less than 0.05; in some embodiments, a correlation is statistically significant when its p-value is less than 0.01. In some embodiments, correlation is assessed by regression analysis. In some embodiments, a correlation is a correlation coefficient.

As used herein, a “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids.

As used herein, the term “subject”, “individual”, or “patient” refers to any organism upon which embodiments of the invention may be used or administered, e.g., for experimental, diagnostic, prophylactic, and/or therapeutic purposes. Typical subjects include animals {e.g., mammals such as mice, rats, rabbits, non-human primates, and humans; insects; worms; etc.). In a preferred embodiment of the invention the subject is a human.

As used herein, a “reporter RNA” or “reporter RNA molecule” or “reporter construct” refers to a molecule that can be cleaved or otherwise deactivated by an activated CRISPR system effector protein described herein. The reporter RNA is configured so that the generation or detection of a detectable signal is not achieved unless the CRISPR effector system is activated. A positive detectable signal may be any signal that can be detected using optical, fluorescent, chemiluminescent, electrochemical or other detection methods known in the art. The reporter RNA may prevent the generation of a detectable positive signal or mask the presence of a detectable positive signal until the reporter RNA is modified by CRISPR effector protein activity. In some embodiments a first signal may be detected when an unmodified reporter construct is present (i.e. a negative detectable signal), which then converts to a second signal (e.g. a positive detectable signal) upon modification of the reporter construct by the activated CRISPR effector protein.

Clustered Regularly Interspaced Short Palindromic Repeats (CRISPR) and CRISPR-associated (CRISPR-Cas) adaptive immune systems contain programmable endonucleases, such as Cas9 and Cpf1 [28,29]. Although both Cas9 and Cpf1 target DNA, single effector RNA-guided RNases have been recently discovered [32] and characterized [31,33]), including Cas13a, providing a platform for specific RNA sensing. Cas12 proteins can also be used to detect target DNA.

RNA-guided RNases can be easily and conveniently reprogrammed using CRISPR guide RNA (gRNAs) to cleave target RNAs. Unlike the DNA endonucleases Cas9 and Cpf1, which cleave only its DNA target, RNA-guided RNases, like Cas13a, remains active after cleaving its RNA target, leading to “collateral” cleavage of non-targeted RNAs in proximity [31].

The embodiments disclosed herein utilize RNA targeting effectors to provide a robust CRISPR-based diagnostic with high sensitivity. Embodiments disclosed herein can differentiate targets from non-targets based on single base pair differences. Moreover, the embodiments disclosed herein can be prepared in freeze-dried format for convenient distribution and point-of-care (POC) applications. Such embodiments are useful in multiple scenarios in human health including, for example, detection of disease-associated non-coding RNA (ncRNA), viral detection, bacterial strain typing, sensitive genotyping, and detection of disease-associated cell free DNA.

In one aspect, the embodiments disclosed herein are directed to a nucleic acid detection system comprising a CRISPR system, a Nanozyme-Linked ImmunoSorbent Assay (NLISA) and one or more guide RNAs designed to bind to corresponding target molecules.

In another aspect, the embodiments disclosed herein are directed to a diagnostic device comprising a CRISPR effector protein, one or more guide RNAs designed to bind to a corresponding target molecule and a catalytic nanoparticle. The device may be a microfluidic based device, a wearable device, or a device comprising a flexible material substrate on which the components are retained.

In another aspect, the embodiments disclosed herein are directed to a method for detecting target nucleic acids in a sample comprising distributing a sample or set of samples into a set of individual discrete volumes, each individual discrete volume comprising a CRISPR effector protein and one or more guide RNAs designed to bind to one target oligonucleotides.

CRISPR Effector Proteins. In general, a CRISPR-Cas or CRISPR system as used in herein and in documents, such as [30], refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus.

In general, a CRISPR system is characterized by elements that promote the formation of a CRISPR complex at the site of a target sequence. Cas13a has been described in the prior art [31,32]. Cas13b has also been described in the prior art [33].

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

The nucleic acid molecule encoding a CRISPR effector protein, in particular Cas13a, is advantageously codon optimized CRISPR effector protein. An example of a codon optimized sequence, is in this instance a sequence optimized for expression in eukaryotes, e.g., humans (i.e. being optimized for expression in humans), or for another eukaryote, animal or mammal as herein discussed. Whilst this is preferred, it will be appreciated that other examples are possible and codon optimization for a host species other than human, or for codon optimization for specific organs is known.

In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded.

In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules.

The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at [34] and these tables can be adapted in a number of ways (see [35]). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

In some embodiments, one or more elements of a nucleic acid-targeting system is derived from a particular organism comprising an endogenous CRISPR RNA-targeting system.

In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of Cas13a or Cas13b orthologs provided herein. In certain example embodiments, the effector protein comprises a single HEPN domain. In certain other example embodiments, the effector protein comprises two HEPN domains.

Additional effectors for use according to the invention can be identified by their proximity to casl genes, for example, though not limited to, within the region 20 kb from the start of the casl gene and 20 kb from the end of the casl gene. In certain embodiments, the effector protein comprises at least one HEPN domain and at least 500 amino acids, and wherein the Cas13a effector protein is naturally present in a prokaryotic genome within 20 kb upstream or downstream of a Cas gene or a CRISPR array.

Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain example embodiments, the Cas13a effector protein is naturally present in a prokaryotic genome within 20 kb upstream or downstream of a Cas 1 gene.

Cas proteins can be prepared in accordance with known methods in the art. For example, Cas proteins can be isolated following recombinant expression in a cell culture system. A Cas protein suitable for implementing the invention can be prepared by any routine method available to the skilled person.

The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related.

In some embodiments, the Type VI RNA-targeting Cas enzyme is Cas13a. In other example embodiments, the Type VI RNA-targeting Cas enzyme is Cas 13b. In particular embodiments, the homologue or orthologue of a Type VI protein such as Cas13a as referred to herein has a sequence homology or identity of at least 30%, or at least 40%, or at least 50%, or at least 60%, or at least 70%, or at least 80%, more preferably at least 85%, even more preferably at least 90%, such as for instance at least 95% with a Type VI protein such as Cas13a (e.g., based on the wild-type sequence of any of Leptotrichia wadei(F0279) Cas13a, Leptotrichia shahii Cas13a, Lachnospiraceae bacterium MA2020 Cas13a, Lachnospiraceae bacterium NK4A179 Cas13a, Clostridium aminophilum (DSM 10710) Cas13a, Carnobacterium gallinarum (DSM 4847) Cas13a, Paludibacter propionicigenes (WB4) Cas13a, Listeria weihenstephanensis (FSL R9-0317) Cas13a, Listeriaceae bacterium (FSL M6-0635) Cas13a, Listeria newyorkensis (FSL M6-0635) Cas13a, Rhodobacter capsulatus (SB 1003) Cas13a, Rhodobacter capsulatus (R121) Cas13a, Rhodobacter capsulatus (DE442) Cas13a, Leptotrichia wadei (Lw2) Cas13a, Leptotrichia buccalis (Lbu) Cas13a, or Listeria seeligeri Cas13a).

In further embodiments, the homologue or orthologue of a Type VI protein such as Cas13a as referred to herein has a sequence identity of at least 30%, or at least 40%, or at least 50%, or at least 60%, or at least 70%, or at least 80%, more preferably at least 85%, even more preferably at least 90%, such as for instance at least 95% with the wild type Cas13a (e.g., based on the wild-type sequence of any of Leptotrichia wadei (F0279) Cas13a, Leptotrichia shahii Cas13a, Lachnospiraceae bacterium MA2020 Cas13a, Lachnospiraceae bacterium NK4A179 Cas13a, Clostridium aminophilum (DSM 10710) Cas13a, Carnobacterium gallinarum (DSM 4847) Cas13a, Paludibacter propionicigenes (WB4) Cas13a, Listeria weihenstephanensis (FSL R9-0317) Cas13a, Listeriaceae bacterium (FSL M6-0635) Cas13a, Listeria newyorkensis (FSL M6-0635) Cas13a, Rhodobacter capsulatus (SB 1003) Cas13a, Rhodobacter capsulatus (R121) Cas13a, Rhodobacter capsulatus (DE442) Cas13a, Leptotrichia wadei (Lw2) Cas13a, Leptotrichia buccalis (Lbu) Cas13a, or Listeria seeligeri Cas13a).

The term “homology,” as used herein, refers to a degree of complementarity. There can be partial homology or complete homology (i.e., identity). A partially complementary sequence that at least partially inhibits a completely complementary sequence from hybridizing to a target nucleic acid is referred to using the functional term “substantially homologous.” When used in reference to a double-stranded nucleic acid sequence such as a cDNA or genomic clone, the term “substantially homologous,” as used herein, refers to a sequence that can hybridize to a strand of the double-stranded nucleic acid sequence under conditions of low stringency. When used in reference to a single-stranded nucleic acid sequence, the term “substantially homologous,” as used herein, refers to a probe that can hybridize to (i.e., is the complement of) the single-stranded nucleic acid template sequence under conditions of low stringency. In some embodiments, sequences described as “homologous” may have an equivalent degree of “identity” to the specified sequence.

“Identical” or “identity” as used herein in the context of two or more nucleic acids or polypeptide sequences means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

### Kits

Diagnostic elements or systems described herein can be provided in a kit. In some instances, the kit includes (a) a container that contains a diagnostic element described herein and, optionally (b) informational material. The informational material can be descriptive, instructional, marketing or other material that relates to the methods described herein and/or the use of a diagnostic element, e.g., for making a diagnosis.

The informational material of the kits is not limited in its form. In some instances, the informational material can include information about production of a diagnostic element, molecular weight of a diagnostic element, concentration, date of expiration, batch or production site information, and so forth. In other situations, the informational material relates to methods of using a diagnostic element, e.g., in a suitable amount, manner, or mode of use (e.g., a method of use described herein).

In some cases, the informational material, e.g., instructions, is provided in printed matter, e.g., a printed text, drawing, and/or photograph, e.g., a label or printed sheet. The informational material can also be provided in other formats, such as Braille, computer readable material, video recording, or audio recording. In other instances, the informational material of the kit is contact information, e.g., a physical address, email address, website, or telephone number, where a user of the kit can obtain substantive information about a diagnostic element therein and/or their use in the methods described herein. The informational material can also be provided in any combination of formats.

In addition to a diagnostic element, the kit can include other components, such as a solvent or buffer, a stabilizer, or a preservative. The kit can also include further elements, e.g., a second or third element, e.g., other diagnostic elements. The components can be provided in any form, e.g., liquid, dried or lyophilized form. The components can be substantially pure (although they can be combined together or delivered separate from one another) and/or sterile. When the components are provided in a liquid solution, the liquid solution can be an aqueous solution, such as a sterile aqueous solution. When the components are provided as a dried form, reconstitution generally is by the addition of a suitable solvent. The solvent, e.g., sterile water or buffer, can optionally be provided in the kit.

The kit can include one or more containers for a diagnostic element or other components. In some cases, the kit contains separate containers, dividers or compartments for a diagnostic element and informational material. For example, a diagnostic element can be contained in a bottle or vial, and the informational material can be contained in a plastic sleeve or packet. In other situations, the separate elements of the kit are contained within a single, undivided container. For example, a diagnostic element can be contained in a bottle or vial that has attached thereto the informational material in the form of a label. In some cases, the kit can include a plurality (e.g., a pack) of individual containers, each containing one or more units of a diagnostic element described herein. The containers of the kits can be air tight, waterproof (e.g., impermeable to changes in moisture or evaporation), and/or light-tight.

### Catalytic Nanoparticles

The catalytic nanoparticles described herein can be used in a method of catalysis. More specifically, the catalytic nanoparticles can be brought into contact with a reactant in a reaction area. The reactant can be any reactant capable of forming a reaction product via a reaction catalyzed by the catalyst of the catalytic nanoparticle. There are various methods of contacting the catalytic nanoparticle with the reactant. In one embodiment, the catalytic nanoparticle can be a heterogeneous catalyst. As such, the catalytic nanoparticle can be immobilized to a solid substrate and a fluid including the reactant can be brought into contact with the catalytic nanoparticle so as to allow the reactant to adsorb onto the catalytic nanoparticle. The fluid can be a gas, a liquid, or a combination thereof. In one embodiment, the catalytic nanoparticle can be fixed to a porous material across which or through which the reactant fluid flows, thus bringing the reactant into contact with the catalytic nanoparticle. In other embodiments, catalytic nanoparticles can form at least a part of a packed reaction bed through with the reaction fluid is passed, thus bringing the reactant into contact with the catalytic nanoparticle.

In other embodiments, the catalytic nanoparticle can be a homogeneous catalyst. In this case, the catalytic nanoparticle can be dispersed in a reaction fluid. As the catalytic nanoparticle is dispersed throughout the reaction fluid it can come into contact with the reactant and catalyse the production of a reaction product in the fluid. In some embodiments, the reaction fluid can be a solution. Due to the stability of the disclosed catalytic nanoparticles, they can be used in a broad range of reaction environments previously limited by support material stability and reaction environment incompatibility. In some embodiments, the solution can have a pH of less than or equal to 14, less than or equal to 13, less than or equal to 12, less than or equal to 11, less than or equal to 10, less than or equal to 9, less than or equal to 8, less than or equal to 7, less than or equal to 6, less than or equal to 5, less than or equal to 4, less than or equal to 3, less than or equal to 2 or less than or equal to 1. In some embodiments, the solution can have a pH of less than or equal to 5, less than or equal to 3, or less than or equal to 1. In other embodiments, the solution can have a pH of greater than or equal to 14, greater than or equal to 13, greater than or equal to 12, greater than or equal to 11, greater than or equal to 10, greater than or equal to 9, greater than or equal to 8, greater than or equal to 7, greater than or equal to 6, greater than or equal to 5, greater than or equal to 4, greater than or equal to 3, greater than or equal to 2 or greater than or equal to 1. In other embodiments, the solution can have a pH of greater than or equal to 8, greater than or equal to 10, greater than or equal to 12, or greater than or equal to 14. In other embodiments, the solution can have a pH of greater than or equal to 8, greater than or equal to 10, greater than or equal to 12, or greater than or equal to 14. In one embodiment, the solution can have a pH of less than or equal to 5. In a preferred embodiment, the solution can have a pH of about 5.

The method of catalysis can also include facilitating a catalytic interaction between the catalytic nanoparticle and the reactant. This can be done in a number of ways. In some embodiments, facilitating a catalytic interaction can include adjusting the pH of the reaction fluid to a suitable pH for catalysis. In some embodiments, facilitating a catalytic interaction can include adjusting a temperature of the reaction fluid to a suitable temperature for catalysis. In some embodiments, facilitating catalytic interaction can include adjusting the ionic strength or tonicity of the reaction fluid. In some embodiments, facilitating a catalytic interaction can include adequately mixing the reaction fluid and/or catalytic nanoparticle to increase the interaction therebetween. In some embodiments, facilitating a catalytic interaction can include directing the flow of the reaction fluid toward and/or across the catalytic nanoparticles. In some embodiments, facilitating a catalytic interaction can include providing a co-catalyst necessary for or beneficial to catalysis of a desired reaction. Other parameters can also be adjusted or employed to facilitate a catalytic interaction between the catalytic nanoparticle and the reactant, such as introduction of electromagnetic radiation, or any other suitable parameter. The catalytic nanoparticles can be used as catalyst in aqueous, polar, or nonpolar solutions.

### EXAMPLES

### Example 1—Materials and Methods

Buffers. RNase-free ultrapure distilled water (UPDW, Invitrogen) was used in all experiments. All buffers were prepared in RNase-free conditions. Phosphate buffer saline (PBS) is 0.01 M phosphate buffer in a 0.8% w/v saline solution, pH 7.5. Coating buffer is a 0.05 M carbonate-bicarbonate buffer, pH 9.6. PBST is PBS with 0.05% v/v Tween 20. Citrate buffer is 50 mM sodium citrate, pH 5.0. The substrate solution contains 0.01% w/v 3,3′,5,5′-tetramethylbenzidine (TMB) and 0.02% v/v H₂O₂ in citrate buffer. 10× Cleavage buffer is 200 mM HEPES, 600 mM NaCl, 90 mM MgCl₂, pH 6.

Pt@Au synthesis. 188 nm Pt@Au was synthesised by further overgrowing Pt onto 120 nm Pt@Au. The 120 nm Pt@Au preparation was described elsewhere [16]. Briefly, 835 μL of UPDW, 165 μL of Pt@Au (120 nm, 500 pM), 20 μL of polyvinylpyrrolidone (PVP, 20% w/v, 10 kDa, Sigma), 40 μL of L-ascorbic acid (100 mg/mL, Sigma), 40 μL of H₂PtCl₆×6H₂O (100 mM, Sigma) were added into a 1.5 mL glass vial in this order of addition. It was vortexed and immediately incubated at 65° C. for 1 h. Pt@Au was then cooled to room temperature, and excess reagents were removed through three sequential washing cycles at 7000 G for 5 min with UPDW. The product was resuspended in UPDW to have a final volume of 165 μL of Pt@Au (500 pM).

Pt@Au characterisation. All batches of Pt@Au were diluted to 6.25 pM in UPDW and characterised using Zetasizer nano series ZEN3600 to measure the charge and particle size based on zeta potential and Dynamic Light Scattering (DLS), respectively. Transmission electron microscopy (TEM) imaging, scanning transmission electron microscopy (STEM) imaging, and energy dispersive X-ray spectroscopy (EDS) analyses were performed on a JEOL JEM-2100F field emission electron microscope operating at 200 kV, equipped with Gatan Orius SC 1000 CCD camera (2 k×4 k), Gatan annular bright field (BF), Gatan high-angle annular dark-field (HAADF), and EDS detectors (Oxford Instruments INCA EDS 80 mm X-Max detector system with STEM capability). Prior to the imaging and analyses, the samples were prepared by placing a 2 μL droplet of the nanoparticle dispersion on a 200-mesh carbon-coated copper grid (Electron Microscopy Science, USA). AZtecTEM Software (Oxford Instruments) was used for all EDS data acquisition and processing, and TruMap™ mode in AZtecTEM was used for the elemental mapping.

Pt@Au functionalisation. Pt@Au was functionalised with streptavidin or neutravidin by mixing 200 μL Pt@Au (500 pM), 20 μL phosphate buffer (50 mM, pH 6.4) and 20 μL of protein (1 mg/mL). The mixture was shaken at 700 rpm for 3 hours at room temperature. 100 μL of blocking protein (beta-casein or BSA at 1 mg/mL) or PBST was added into the mixture. It was then shaken at 700 rpm for 1 hour at room temperature. Excess reagents were removed through three sequential washing cycles at 7000 G for 5 min with PBST. The product was resuspended in PBST to have a final volume of 200 μL of streptavidin-Pt@Au or neutravidin-Pt@Au (500 pM).

Nanozyme-linked Immunosorbent Assay (NLISA). A microtiter plate (384 wells, Maxisorp™, Nunc) was coated with anti-FAM antibody (100 ng/mL in coating buffer, 40 μL per well, Abcam) for 3 h at r.t. and covered with an adhesive plate sealer. The plate was washed three times with PBST (100 μL per well), and 28 μL of PBST were added per well followed by the solution containing the reporter RNA (5′-FAM-UUUUUC-Biotin-3′, from 10 nM to 10 fM and 0 in PBST, 14 μL per well) or the LwaCas13a reaction mixture (14 μL per well). After 30 min at r.t., the plate was washed as before, and a solution of streptavidin-Pt@Au (0.5 pM in PBST, 40 μL per well) was added to the wells and incubated for 30 minutes at r.t. The plate was washed again, and the substrate solution was added (40 μL per well). Colour development was stopped after 30 min at r.t. with 4 N H₂SO₄ (20 μL per well). Signal readout was plotted based on absorbance at 450 nm (Spectramax M5, Molecular Devices) or central pixel blue-intensity of each well measured using image software (FIJI) of a photo of the plate (Surface Pro 4 rear camera, 8 MP).

Target RNA. Synthetic target RNA was supplied by Integrated DNA Technologies. The RNA used in this study is summarised in (FIG. 12). rt-RPA primers are complementary to the RNA target sequence. They were designed such that the forward primer is at the 5′ end of the target site that is bound by the gRNA's spacer, while the reverse primer is at the 3′ end. The forward primers also contain a T7 promoter sequence, enabling DNA amplicons to be first transcribed to RNA prior to Cas13-based detection. The primers were ordered as DNA (FIG. 13, Biomers). LwaCas13a gRNAs contained 28 nucleotide-long spacers complementary to the target site.

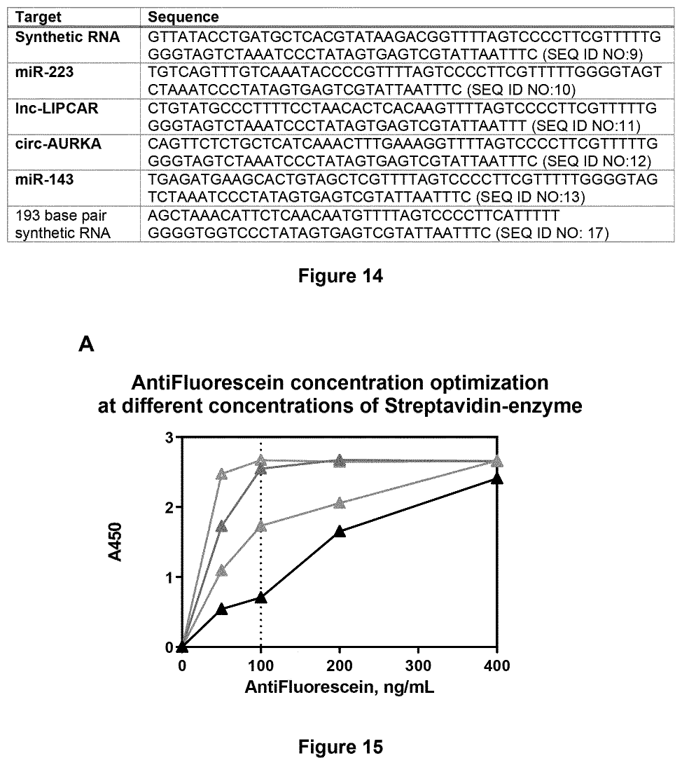

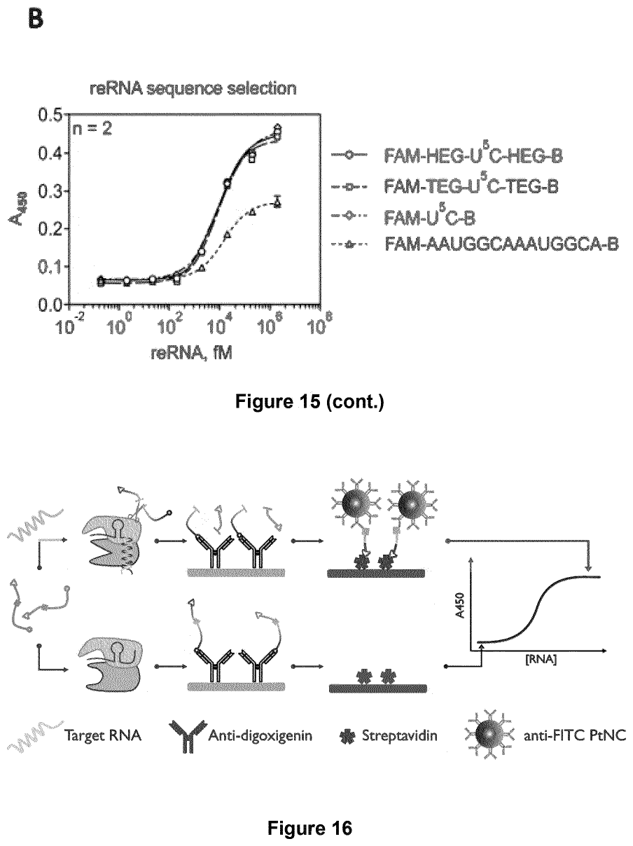

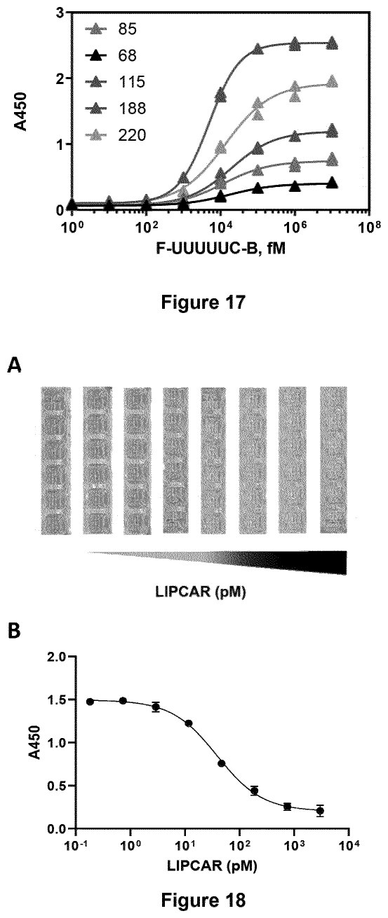

For miRNA targets, the spacers were designed to include the entire miRNA length. Constructs were ordered as DNA (FIG. 14, Biomers/Eurofins) with an appended T7 promoter sequence for in vitro RNA transcription.

gRNA production. Synthesis of gRNAs was done using the HiScribe T7 Quick High Yield RNA Synthesis kit (New England Biolabs) according to the manufacturer's instructions and purified using the Monarch® RNA Cleanup Kit (50 μg, New England Biolabs). DNA oligonucleotides containing a T7 promoter sequence served as templates. gRNA purity was checked using Bioanalyzer (Agilent) Small RNA Analysis Kit following the supplier's protocol.

LwaCas13a reaction. A Master Mix was prepared with a concentration of 3× of Cleavage buffer, 1.5 U/μL Murine RNase Buffer (New England Biolabs), 375 nM of RNase Alert V2 (Thermo Fisher Scientific), 135 nM of LwaCas13a (Genscript) and 67.5 nM gRNA. 10 μL of synthetic RNA standards (from 1 μM to 10 pM and 0, prepared in 1 ng/μL of PolyA RNA carrier) or samples were mixed with 5 μL of Cas13 reaction Master Mix in a 384-well black clear bottom plate (Corning) and let to react for 3 h at 25 or 37° C. Fluorescence was measured at 490/520 nm each 5 minutes for 3 h (Spectramax M5, Molecular Devices).

When combined with NLISA (CrisprZyme), the Master Mix was prepared with a concentration of 3× of Cleavage buffer, containing 1.5 U/μL Murine RNase Buffer (New England Biolabs), 0.75 nM of 5′-FAM-UUUUUC-Biotin-3′ (Integrated DNA Technologies), 300 nM of LwaCas13a (GeneScript) and 360 nM gRNA. 10 μL of synthetic RNA standards (from 2000 pM to 1.5 pM and 0, prepared in 10 ng/μL of PolyA carrier) or samples were mixed with 5 μL of Cas13 reaction Master Mix in a 384-well PCR plate (Thermo Fisher Scientific) and let to react for 3 h at r.t. 14 μL were then provided as input for the NLISA. Reactions with LbuCas13a were performed similarly, with final concentrations of 10 mM Tris-HCl buffer, 10 mM NaCl, 1.5 mM MgCl2, 1 U/μL Murine RNase Buffer (New England Biolabs), 1.25 ng/μL HEK293T RNA, 0.1 nM of 5′-FAM-UUUUUC-Biotin-3′ (Integrated DNA Technologies), 100 nM of LbuCas13a and 65.5 nM gRNA.

Lateral flow assay (LFA). The lateral flow strips with antiFITC test line were produced using an automated liquid dispenser (BioDot System AD3220). 0.5 mg/mL of filtered (0.2 uM filter) anti-FITC antibody (Abcam 19224, lot GR175456-62) was dispensed at a height of 5 mm from the bottom of the nitrocellulose (CN95 Unisart@Nitrocellulose Membrane, Sartorius) before being dried overnight at 37° C. Lateral flow half-dipstick assays were then assembled onto backing card (Kenosha, KN-PS1060.19) with overlapping absorbent pad material (Ahlstrom-munksjo, KN-222-20.1), before being cut into 4 mm wide test strips.

The LFAs were run in half-dipstick format by dipping the test strips into a 96-well plate (Corning #3641, flat bottom, non-binding surface) containing 10 μL of Cas reaction product, 50 μL PBST, 10 μL of streptavidin-functionalised Pt@Au (1 pM, blocked with beta casein). After the solution had fully wicked up the strip (ca. 12 min), the strip was then dipped in another well containing 100 μL of PBST for 10 min to wash through any unbound nanoparticles or reporter RNA. Subsequently, the strip was submerged in another well for 10 min filled with 330 μL (enough solution to cover test line on strip in well) freshly prepared PtNC substrate solution containing 1× Pierce™ CN/DAB (4-chloro-1-naphthol/3,3′-diaminobenzidine, tetrahydrochloride) Substrate Kit (Thermo Scientific), 12% (w/w) hydrogen peroxide (Sigma), 50% (v/v) stable peroxide buffer. Finally, the strip was moved into a well containing 330 μL purified water for 10 min to stop the reaction and was dried under ambient condition for 5 min. Strips were imaged with an iPhone XS mobile phone camera. Test line intensities were quantified using ImageJ by first converting the image to grayscale (32 bit) before drawing a rectangle the width of the lateral flow strips and length long enough to include an internal control of one of the background grid lines. Using the gel analyzer tool, the pixel density of each test line was integrated before being normalised relative to the pixel density of the photo grid.

Real-time Recombinase Polymerase Amplification (rt-RPA)—rt-RPA was performed using the TwistAmp Basic Kit (TwistDx) as per the manufacturer's instructions and with the following modifications. The total reaction volume was 20 μL. First, 3.08 μL of RNA target and 1.92 μL of a 10 μM 1:1 mix of forward and reverse primers were heated for 10 min at 65° C. A master mix was then prepared for the resuspension of TwistAmp Basic reaction pellets, where each pellet is to be resuspended with 29.5 μL Rehydration buffer, 2.05 μL UPDW, 0.95 μL Dithiothreitol (Sigma-Aldrich) at 1 M, 2.5 μl GoScript reverse transcriptase (Promega) at 160 U/μL, and 2.5 μL MgAOc at 280 mM. 15 μL of the resuspended TwistAmp Basic reactions was added to each 5 μL RNA target-primer mixture, resulting in forward and reverse primers at 480 nM, Dithiothreitol at 19 mM, and GoScript reverse transcriptase at 8 U/μL. Each reaction was incubated at 42° C. for 60 min.

Real-time quantitative polymerase chain reaction (rt-qPCR). miScript II RT Kit (Qiagen) was used for reverse transcription and miScript SYBR Green PCR Kit (Qiagen, miScript Primer Assay MS00003871) for qPCR following the supplier's protocol. Reverse transcription was performed with 10 μL of input, synthetic RNA standards (from 1000 to 0.1 fM and NTC, prepared in 1 ng/μL of PolyA carrier) or samples in 0.2-mL PCR tubes. qPCR was performed with 1 μL as input in a 384-well PCR-plate on a QuantStudio™ 6 cycler (ThermoFisher). Samples were interpolated using standard regression and samples with a concentration of miR-223>1 pM were selected and analysed by CrisprZyme.

Cell culture and cell differentiation. The human episomal iPSC line (Thermo Fisher) was maintained in the complete Essential 8™ medium (Thermo Fisher) on 6-well plates coated with Matrigel© (Corning) diluted in DMEM/F12 (Thermo Fisher). The cells were regularly split at 1:12 ratio every 4 days using 0.5 mM EDTA. The medium was supplemented with 10 μM Rock inhibitor Y-27632 (STEMCELL Technologies) for the first 24 h after passaging to avoid cell dissociation-induced apoptosis. Cardiac differentiation was optimised from the previously reported protocol [36] as follows: the iPSC medium was changed to the differentiation medium, RPMI supplemented with 2% v/v B27-insulin supplement (Thermo Fisher), 4 days after the split, when cells reached˜85% confluence. From day 0 to day 2, the differentiation medium was supplemented with 6 μM CHIR99021 (tebu-bio) and replaced with the fresh differentiation medium on day 2.

From day 3 to day 5, the differentiation medium supplemented with 2.5 μM Wnt-C59 (Stratech) was applied. On day 5, medium was replaced with the fresh differentiation medium. Medium was switched to RPMI supplemented with 2% v/v B27 supplement (Thermo Fisher) on day 7 and replaced with fresh medium every other day. Spontaneous contraction of the cells was observed from day 7. Medium was switched to the RPMI without glucose supplemented with 2% v/v B27 supplement (Thermo Fisher) and 5 mM sodium lactate (Merck) and changed every other day for the metabolic selection of cardiomyocytes from day 11 to day 17. ncRNA was extracted on day 17 following the protocol described below.

Patient cohort. Plasma samples for the evaluation of Inc-LIPCAR were obtained from discarded material from clinical samples initially obtained from adults (>18 years) presenting to Massachusetts General Hospital with chest pain and tested for high-sensitivity Troponin T (hsTnT). The material was excess to clinical needs and selected based on hsTnT values and storage at 4° C. for <12 hours after initial blood draw. Plasma was then frozen at −80° C. prior to research use. The study was granted exemption from informed consent due to the use of anonymized discarded clinical samples and was approved by the Mass General Brigham IRB (Protocol #: 2019P002499).

RNA specimens extracted from tissue biopsies for the measurement of circ-AURKA were obtained through the Prostate Cancer Biorepository Network (PCBN), a US Department of Defense (DOD)/Congressionally Directed Medical Research Program (CDMRP) bioresource. All samples were de-identified (MIT exempt determination E-1564). Primary adenocarcinoma samples were provided by Johns Hopkins University and neuroendocrine castration-resistant prostate cancer samples were provided by the University of Washington through RNA extraction of metastatic cancer tissue.

Non-coding RNA (ncRNA) extraction from samples. ncRNA was extracted from a cell pellet or 200 μL of blood using the miRNeasy Micro Kit (Qiagen) following the supplier's protocol. 3.5 μL of the Spike-In Control (Ce-miR-39, 1.6×10⁸ copies/μL) was added after the QIAzol Reagent. ncRNAs were eluted from the RNeasyMinElute spin column with two washes of 14 and 8 μL of UPDW (total RNA>30 ng/μL, A260/280>1). ncRNAs were diluted to 2.5 ng/μL of total RNA to be used as input for rt-RPA (3.08 μL) and LwaCas13a reaction (10 μL).

Data and statistical Analysis. All data analysis was conducted in GraphPad 9.0.0 (Prism). All the sample sizes and statistical tests are specified in the figure legends. Calibration curves were fitted to a four-parameter equation according to the following formula: Y=Bottom+(Top-Bottom)/(1+10\hat{ }((Log EC50−X)*HillSlope)), where Top is the maximum signal, Bottom is the minimum signal, Top and Bottom are plateaus in the units of the Y axis, EC50 is the concentration producing 50% of the maximal signal, and Hillslope is the slope at the inflection point of the sigmoid curve. Limit of detection (LOD) was defined as the 10% signal of the Bottom signal. Results were normalized defining 0% as the smallest mean in each data set and 100% as the largest mean in each dataset.

### Example 2—Nanozyme Functionalisation and Characterisation

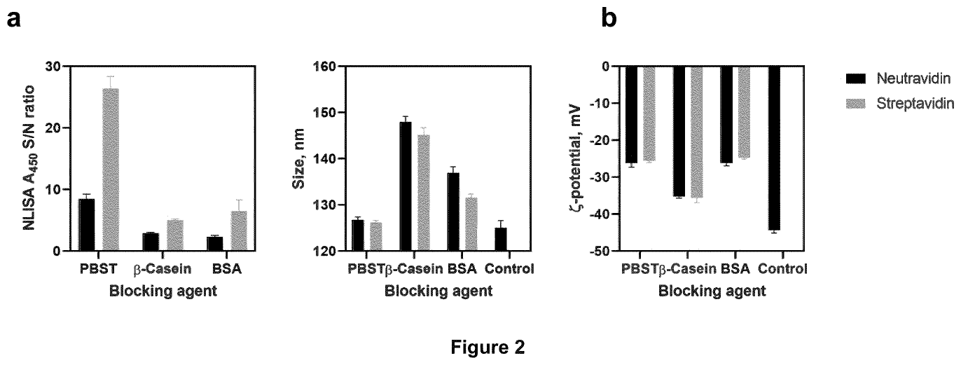

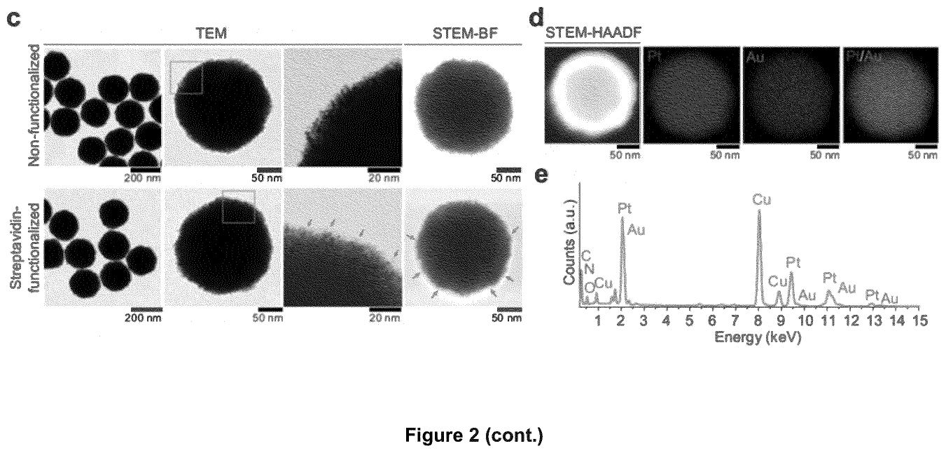

Platinum and gold (Pt@Au) nanozymes were chosen due to their higher catalytic activity when compared to other enzymes and nanozymes [16]. Briefly, Pt@Au were prepared by overgrowing platinum on a 15 nm gold nanoparticle a seed, in the presence of polyvinylpyrrolidone (PVP) and L-ascorbic acid which function as a stabiliser and a reducing agent for the platinum salt, respectively [16](FIG. 2a).

To be able to bind the particles to on end of the reporter RNA, where biotin is present, Pt@Au particles were functionalised with a biotin-binding protein to be able to bind them to the biotinylated reporter RNA. First, the pH, the protein concentration and the blocking agents of the functionalization step were studied to reach the optimal binding meaning the highest signal to noise (S/N) ratio in the NLISA. different functionalisation procedures were tested for optimal binding through the variation of pH, protein concentrations and blocking agents. It was found that pH 6.4-6.5 showed a higher ratio (FIG. 6), slightly above the isoelectric point of the biotin-binding protein (5 and 6.3 for streptavidin and neutravidin, respectively).

Next, we aimed to improve the S/N ratio further by testing different biotin-binding proteins and blocking agents (FIG. 2b). A combination of the biotin-binding protein streptavidin combined with phosphate-buffered saline solution containing 0.05% v/v Tween20 (PBST) without any blocking protein was selected. Protein blocking through addition of beta-casein or bovine serum albumin (BSA) was not needed to avoid any non-specific interaction on the NLISA plate.

The particles were characterised by dynamic light scattering (DLS) to evaluate the functionalised particle characteristics. Polydispersity index (PDI) was reported to be below 0.074, showing that the particles are highly monodisperse.

Furthermore, the ζ-potential change, from −44.3 mV to −25.6 mV, confirmed the adsorption of the biotin-binding proteins on the surface of the Pt@Au. Only the blocking with beta-casein showed a different surface potential, suggesting that beta-casein surface coverage dominates over the biotin-binding protein. Transmission electron microscopy (TEM) and scanning transmission electron microscopy (STEM) brightfield (BF) images showed spherical nanoparticles of ca. 200 nm in diameter with nanometer-sized pores (ca. 1-2 nm) throughout the surface (FIG. 2c, FIG. 7).

The nanoscale pores are selectively accessible to small molecules (e.g., H₂O₂) and significantly increase the surface area for catalytic amplification, as we previously reported [16]. On the other hand, functionalising with streptavidin did not induce any noticeable change to the overall morphology and size of Pt@Au. Notably, however, it was observed that a thin layer of amorphous substance on the surface of Pt@Au was formed, which presumably indicates the successful functionalisation of the protein layer on the surface (FIG. 2c).

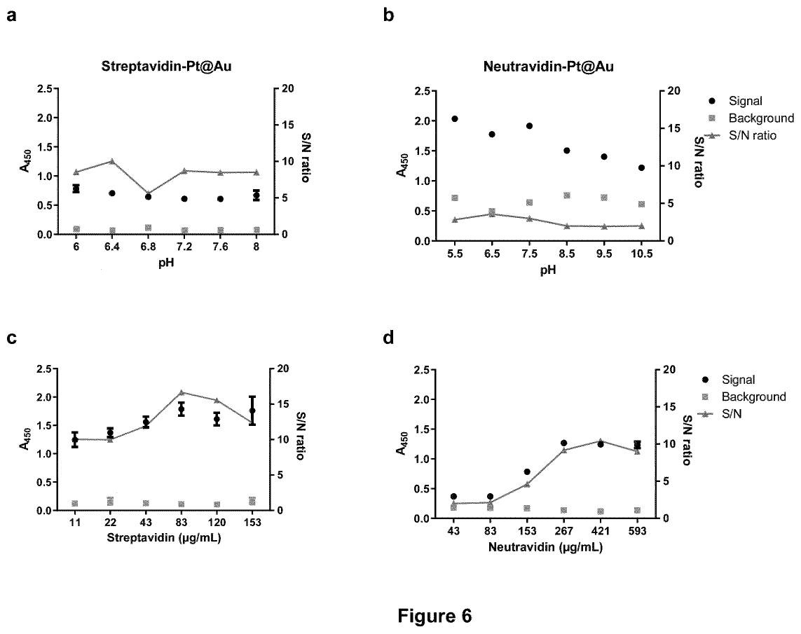

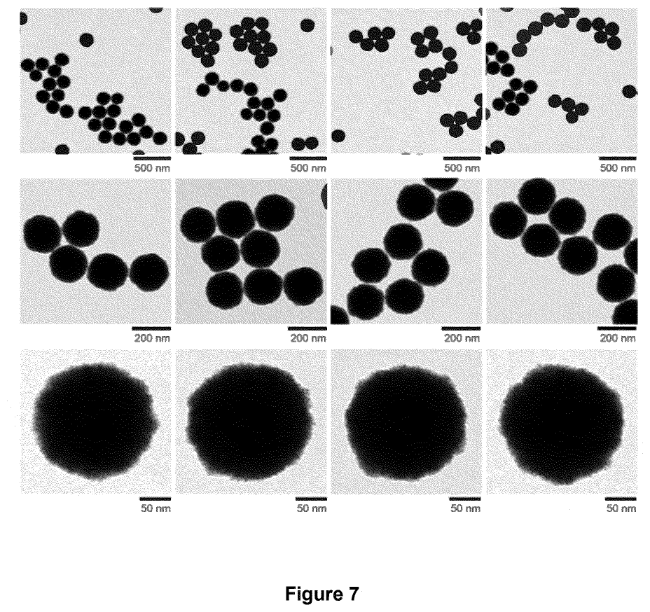

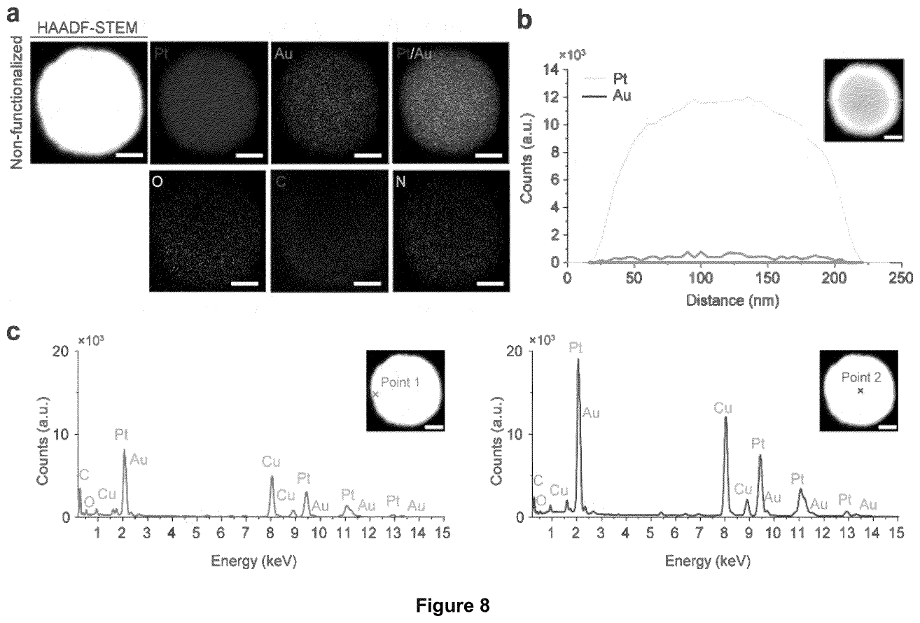

High-angle annular dark-field STEM (HAADF-STEM) was performed that shows the atomic (Z) number contrast and applied energy dispersive X-ray spectroscopy (EDS) to analyse the elemental distribution in Pt@Au and streptavidin-Pt@Au (FIG. 2d-2e, FIG. 8). The EDS elemental maps and intensity point/line profile analyses revealed that Pt and Au were present throughout the inner particle while Pt was the main element found in the periphery region, suggesting a growth mechanism involving multiple Au seeds and overgrown Pt layers.

### Example 3—Optimisation of the NLISA

Once the streptavidin-Pt@Au functionalization was optimized. The nanozymes were incorporated into the NLISA assay design. This assay consisted of four sequential steps: the addition of (1) antibodies directed against 6-carboxyfluorescein (anti-FAM) to the plate, (2) the reporter RNA with two functional handles (a FAM and a biotin molecule at each end, 5′-FAM-UUUUUC-Biotin-3′), (3) streptavidin functionalised nanoparticles (streptavidin-Pt@Au), and (4) a final addition of the chromogenic substrate, 3,3′,5,5′-Tetramethylbenzidine (TMB), for colour development.

TMB is a suitable colourless chromogenic substrate that becomes coloured when oxidized by Pt@Au nanozymes which use hydrogen peroxide as a cofactor. The coloured substrate can be protonated by the addition of diluted sulfuric acid resulting in a yellow solution with absorption maxima at 450 nm. Between each of the described steps, an extra wash step is performed with buffer, except after addition of the chromogenic substrate.

The complex formed between the anti-FAM and streptavidin-Pt@Au through the bridge created by the reporter RNA determines the reporter RNA concentration.

In this study, a four-parameter sigmoidal regression curve was used to process the data where the concentration of analyte is in log scale. The concentration of analyte is defined as the concentration of target RNA in a sample, to allow comparison of the concentrations reported here with other publications. This regression, Equation 1, can define the dynamic range of the assay between 10% (EC₁₀) and 90% (EC₉₀) of the maximum and minimum signal (Top and Bottom). The hillslope describes the steepness and indicates the quantification capability, where a value close to 1 enables the best quantification accuracy:

$$Y = Bottom + \frac{Top - Bottom}{1 + {10}^{( LogEC 50 - X ) \times HillSlope}} \qquad Equation 1$$

This equation can alternatively be expressed as:

$$Equation 1 ( alt . labels )$$

$$Abs = {Signal}_{min} + \frac{{Signal}_{max} - {Signal}_{min}}{1 + {10}^{( LogEC 50 - Log ( concentration ) ) \times HillSlope}}$$

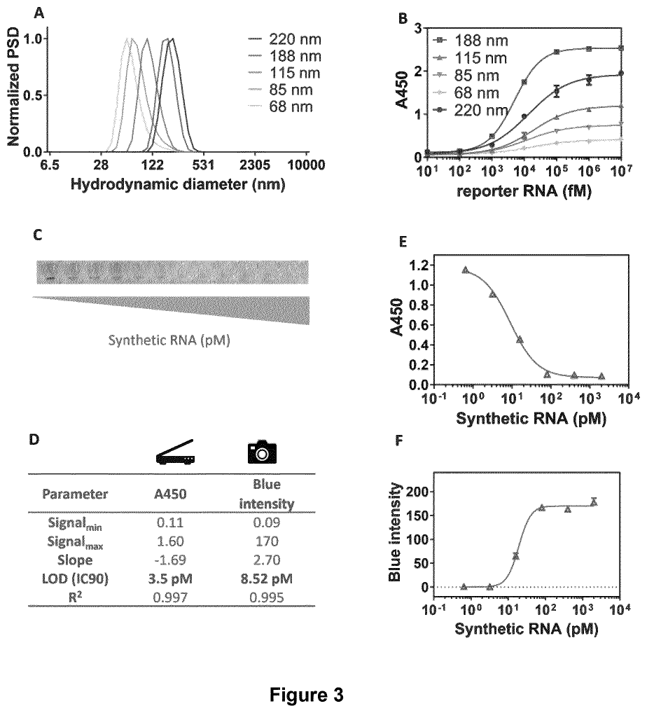

To achieve the lowest limit of detection (LOD) of the reporter RNA concentration, calculated as EC₁₀, the effect of nanozyme size was tested. We first overgrew different amounts of Pt onto the 15-nm Au seeds to prepare particles with a range of diameters from 68 to 220 nm (FIG. 3a). Then, a two-dimensional titration assay was conducted to determine the required concentration of anti-FAM to form a single monolayer of immunoreagent and streptavidin-Pt@Au to provide an absorbance of ˜1, selected arbitrarily outside the absorbance saturation range of the chromogenic substrate solution. We found that 100 ng/mL of anti-FAM and 333 fM of streptavidin-Pt@Au was the best combination.

We also evaluated the sensitivity for the reporter RNA using the different particle sizes (FIG. 3b). We observed that a bigger particle size resulted in higher absorbance without compromising the LOD. Bigger particle size enables a larger catalytic surface area per binding event, thus increasing the detection capability. Using 188 nm particles, we achieved a sensitivity for the reported RNA of 550 fM. These optimized conditions were used in the experiments described below.

Four-parameter sigmoidal regression curve where the concentration of analyte is in log scale. The concentration of analyte is defined as the concentration of target RNA in a sample, to allow comparison of the concentrations reported here with other publications. This regression, Equation 2, can define the dynamic range of the assay between 10% (EC10) and 90% (EC90) of the maximum and minimum signal. The hillslope describes the steepness and indicates the quantification capability, where a value close to 1 enables the best quantification accuracy.

In some embodiments, Equation 1 is used to determine the dynamic range of the assay, for example between 10% (EC₁₀) and 90% (EC₉₀) of the maximum and minimum signal (Top and Bottom).

### Example 4—RNA Quantification Using CrisprZyme

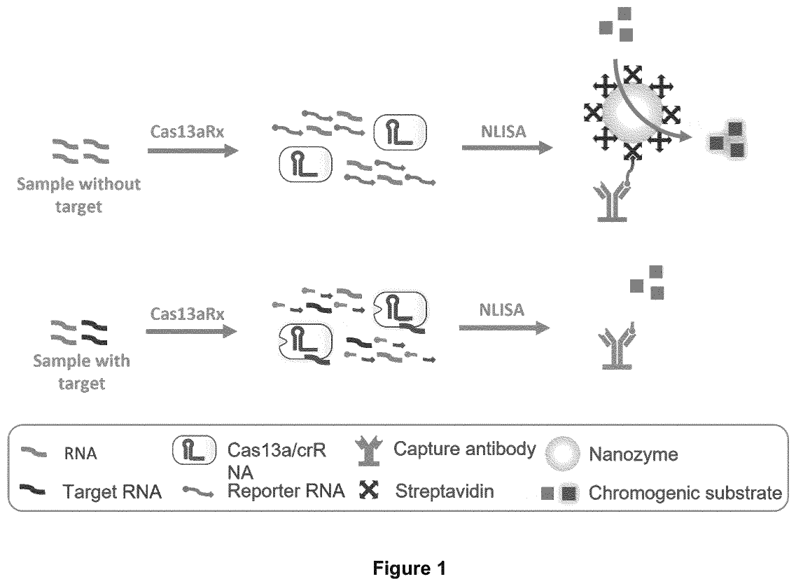

To develop a sensitive assay for the detection of target RNA, we combined the optimized procedure for the reporter RNA quantification by NLISA with the Cas13-based reaction (CrisprZyme, FIG. 1). We performed the optimization experiments with a synthetic 71-nucleotide RNA as a target analyte.

Briefly, a mastermix solution that contains Cas13 from Leptotrichia wadeii (LwaCas13a), gRNA, and reporter RNA, was mixed with the target RNA to trigger the cleavage of the reporter RNA. The reaction product which contained different levels of cleaved reporter RNA depending on the presence of target RNA was then added to the NLISA to quantify the amount of cleaved reporter RNA.

NLISA is able to quantify the amount of cleaved reporter RNA since the cleavage of reporter RNA prevents the binding between the anti-FAM antibody and the streptavidin functionalized nanozyme. The lack of binding reduces the linking of nanozymes particles to the solid support, unbound particles are removed during the washing step. This reduction in the number of nanozymes resulted in a lower catalytic activity, and less chromogenic substrate oxidation. In conclusion, the presence of target RNA triggers reporter RNA cleavage which prevents the binding of the nanozymes and inhibits colour development, whereas the absence of target allows the nanozyme-triggered colour change.

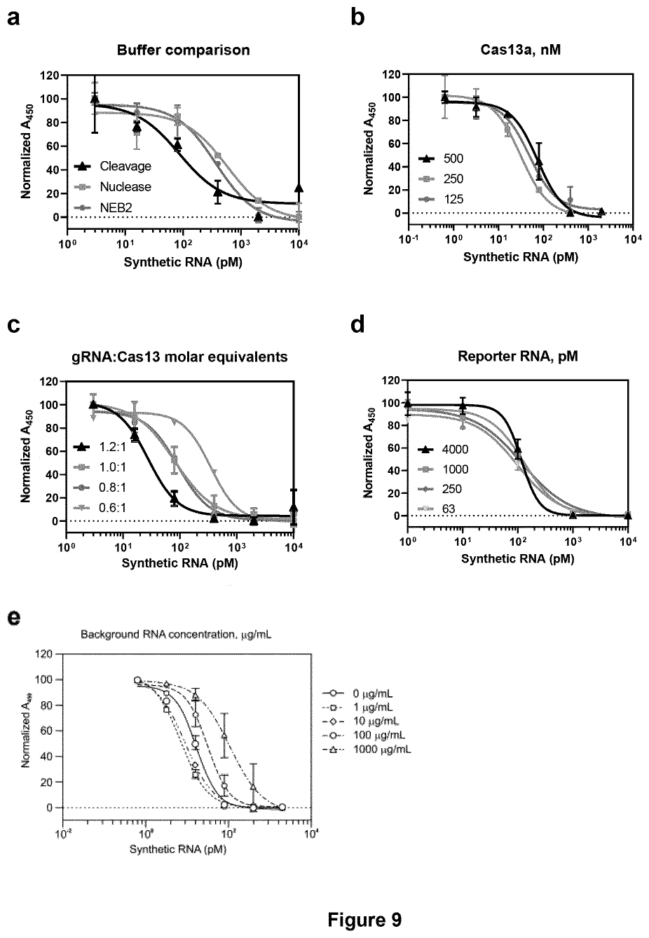

We evaluated all the components of the reaction including buffers, enzyme and reporter concentrations (FIG. 9) to obtain the best LOD. We observed that the best reporter RNA concentration was 0.75 nM, which is more than two orders of magnitude lower than the concentration in the mastermixes reported in the literature [37]. This is due to the high sensitivity of NLISA, which allows for quantification of reporter RNA at lower concentrations than the fluorescent readout.

We also tested the ratio of gRNA to Cas13, results showed that a molar ratio of 1.2:1 (gRNA:Cas13) is able to improve the sensitivity by 10-fold.

The optimised CrisprZyme assay colorimetric readout can be observed by naked eye (FIG. 3c). Quantification of the colour change with measuring the absorbance at 450 nm (A₄₅₀) with a spectrometer showed an LOD of 3.5 pM (FIG. 3d,e), while image analysis of the blue intensity in the central pixel of each well resulted in an LOD of 8.5 pM (FIG. 3d,f). While the latter allowed for analysis with only a camera, a higher slope of the regression curve reduced the accuracy of the system to a semi-quantitative level.

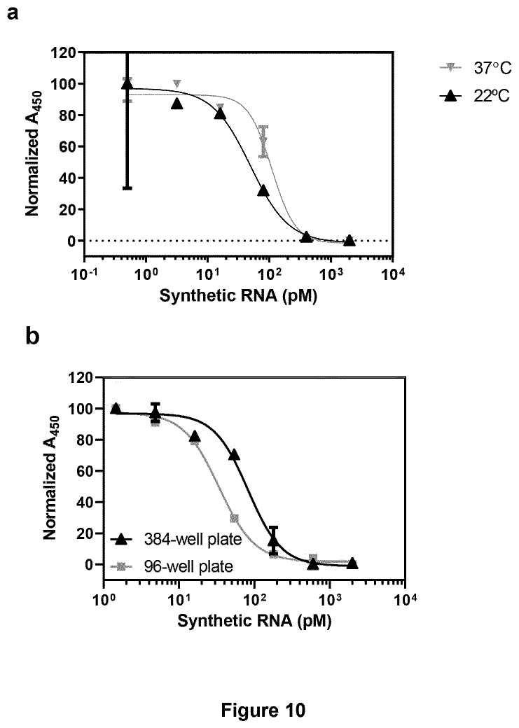

To confirm assay versatility in resource-limitted settings, we next tested whether CrisprZyme could be conducted at room temperature, overcoming the need for thermal cyclers or heating devices. Performing CrisprZyme at room temperature (˜22° C.) resulted in a similar LOD as incubation at 37° C. (FIG. 10a). Furthermore, the assay also showed comparable results in both 384- and 96-well plates (FIG. 10b), allowing for high-throughput applications.

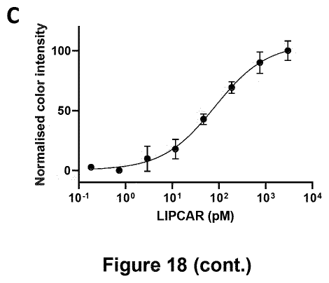

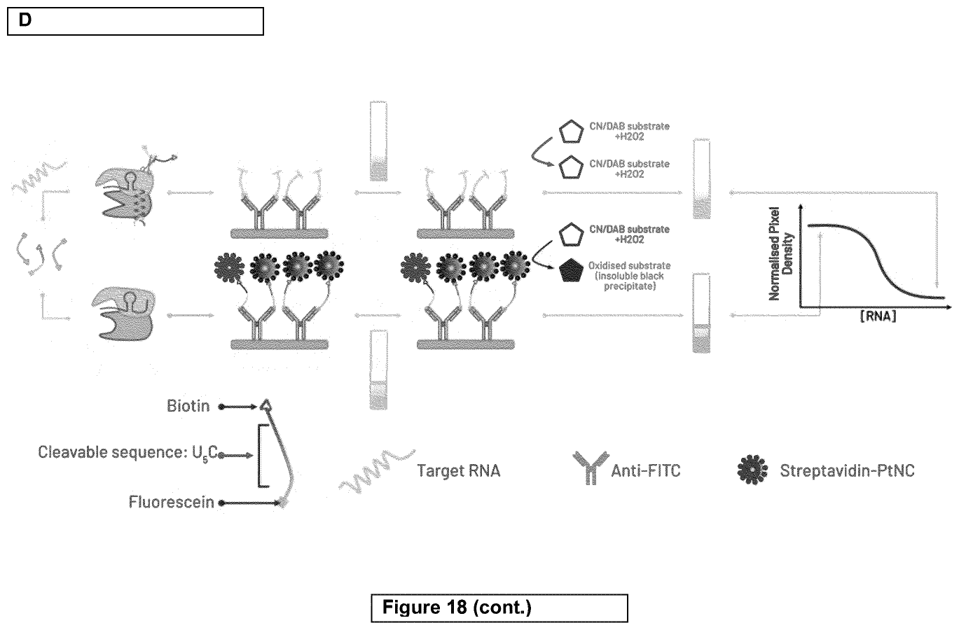

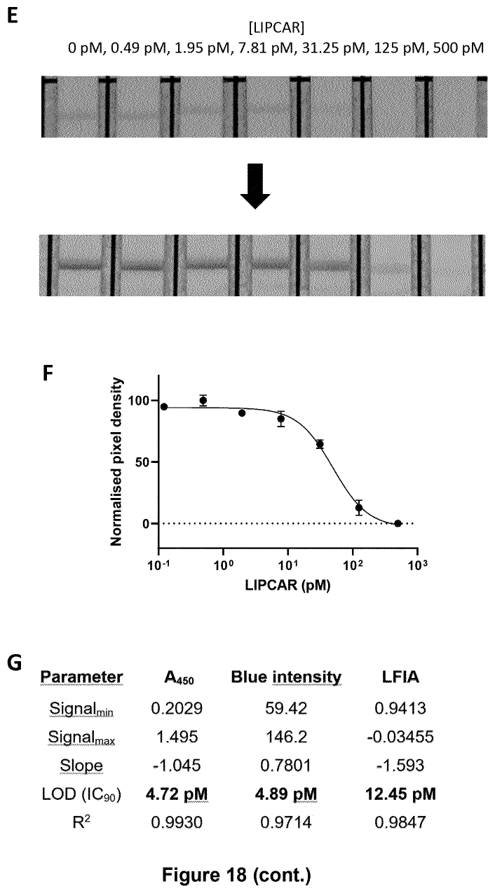

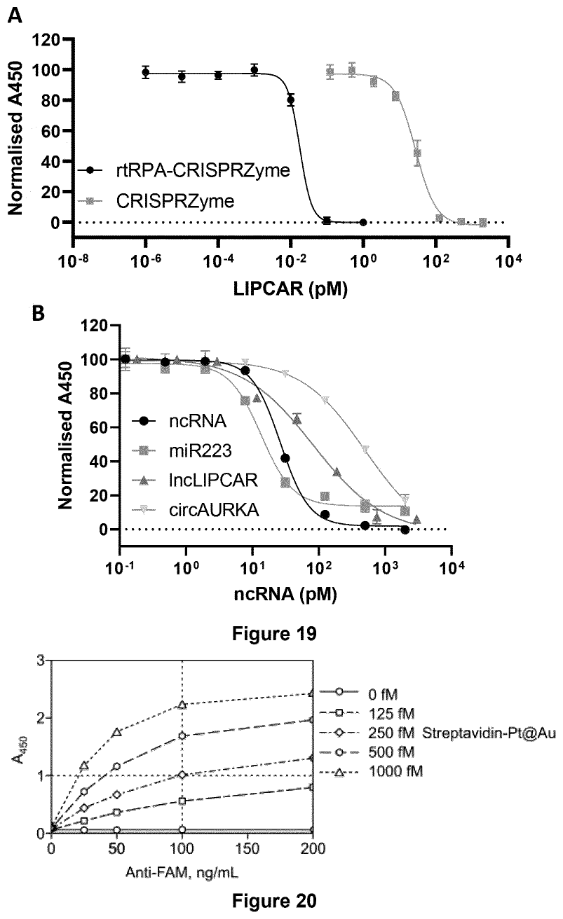

Quantification of the colour change with a plate reader showed an LOD of 4.72 pM through the measurement of absorbance at 450 nm (A₄₅₀) (FIG. 18b,g), while image analysis of the blue intensity in the central pixel of each well resulted in an LOD of 4.89 pM (FIG. 18c,g). Importantly, all LwaCas13a-based CrisprZyme assays were fully run at ambient temperature. Next, we adapted CrisprZyme for the readout with a lateral flow assay (LFA) (FIG. 18d). Target RNA is mixed with the gRNA-Cas13 complex and reporter RNA in the CRISPR reaction. Subsequently, streptavidin-functionalised nanozymes are mixed with the CRISPR reaction product containing the biotinylated reporter RNA to form a complex. A test strip preprinted with anti-FAM composed of nitrocellulose membrane and an absorbent pad was used to draw the mixture driven by capillary action. This allowed the capture of the non-cleaved reporter RNA-nanozyme complex on the test line. When exposed to the substrate solution, the nanozymes on the test line catalysed the substrate oxidation generating an insoluble black line. By quantifying the pixel density on the test line on the image taken using a smartphone (FIG. 18e), we achieved a LOD of 12.5 pM (FIG. 18f, g). It is noteworthy that β-casein blocking on the particles is required to minimise the non-specific binding on the LFAs.

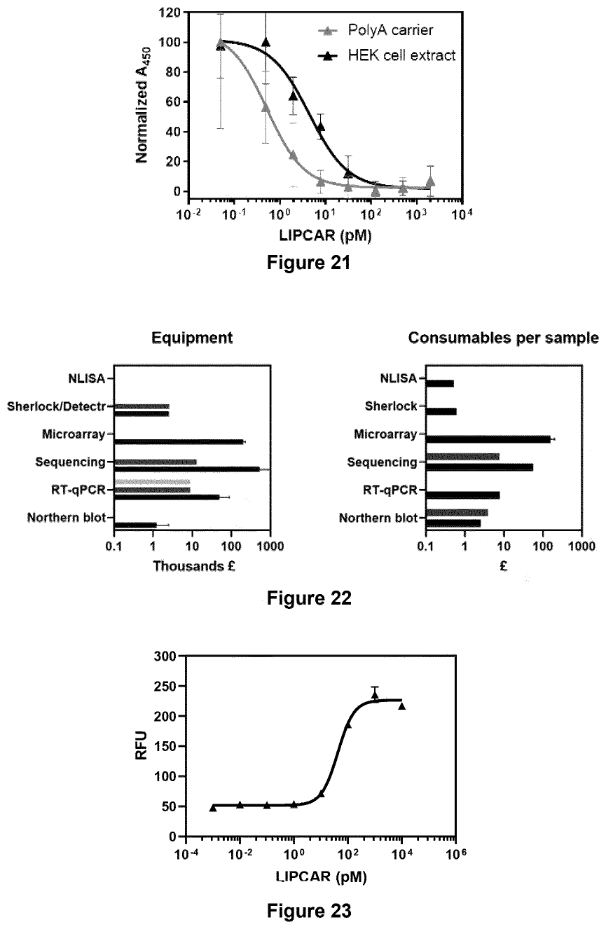

To confirm the assay's simplicity, we next tested whether CrisprZyme could be run in both 96- and 384-well plates (FIG. 10b), allowing for high-throughput applications. The presence of non-targeting RNA strands in the background HEK RNA (FIG. 21) resulted in a decrease in sensitivity of one order of magnitude. The assay time of the nanozyme part of CrisprZyme was dependent on the readout technology ranging between 30 min for lateral flow to 90 min for the plate reader format (FIG. 26). Combined with the Cas reaction (3 h) the total assay time was 3.5 h for lateral flow or 4.5 h for the plate reader. Further work should address shorter Cas reaction times followed by nanozyme-based readouts. Also, a full comparison of molecular diagnostic technology price (FIG. 23) shows that our technology compares in consumable price to SHERLOCK and DETECTR strategies, without the requirement of purchasing small equipment.

### Example 5—Expanding the Dynamic Range of Cas13-Based Diagnostics

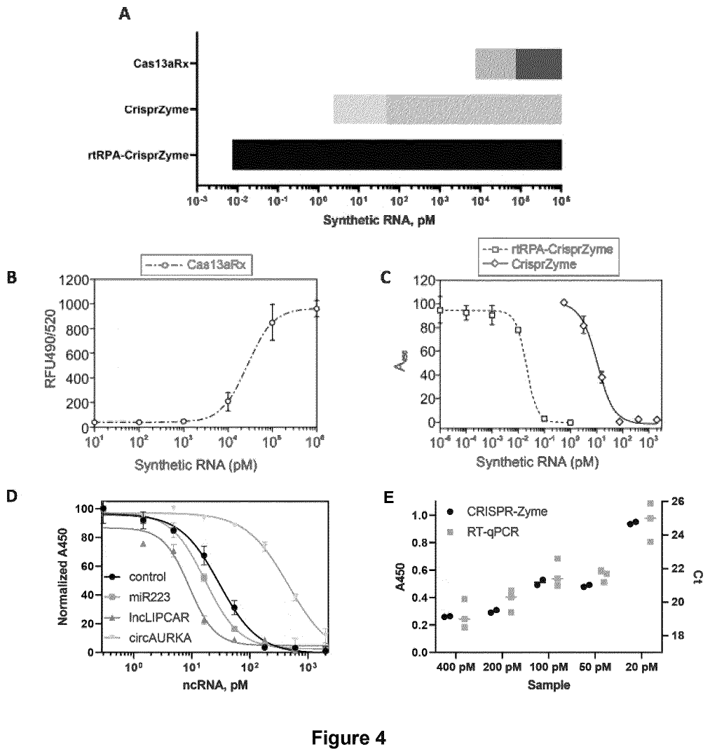

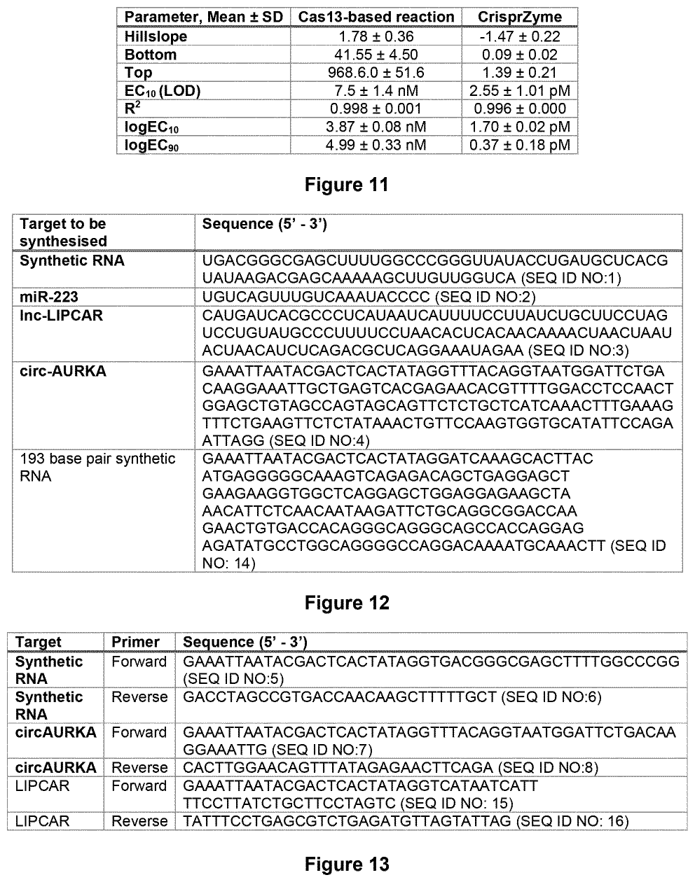

To understand the enhancement of signal produced by the introduction of the NLISA we tested the Cas13-based assay for their dynamic range (FIG. 4a). The conventional fluorescence-based Cas13 reaction using fluorescence readout reached an LOD of 7.5±1.4 nM, and its standard curve showed a steep slope (1.88±0.36), consistent with previously reported results [38](FIG. 4b, FIG. 11).

In contrast, CrisprZyme showed an improved LOD of 2.55±1.01 pM, three orders of magnitude lower than the Cas13 reaction alone (FIG. 4c, FIG. 11), which is comparable to other amplification-free strategies that require a microfluidic sensor with electrochemical readout [6]. Furthermore, the shallower slope of CrisprZyme, −1.47±0.22, allows for the use of this technology for accurate quantification, rather than only qualitative detection.

To further improve the LOD of the assay, we combined CrisprZyme with a preamplification step as described in the SHERLOCK technology [2]. We included reverse transcription recombinase polymerase amplification (rt-RPA), followed by T7-based RNA transcription, Cas13 detection and NLISA. This further enhanced the sensitivity as compared to Cas13 alone up to six orders of magnitude to an LOD of 7.61±1.36 fM (FIG. 4c, FIG. 11).

However, due to exponential target amplification through rt-RPA, the slope was steeper (−2.13±0.08) limiting its use in quantitative RNA detection and non-communicable diseases. Furthermore, despite the sensitivity enhancement of the NLISA for preamplification-free RNA detection, we discovered that the LOD of the preamplification-based assay is determined by rt-RPA's capability to create copies of the target analyte and inclusion of the NLISA thus does not necessarily reach a lower LOD.

Given the high dynamic range and low LOD of CrisprZyme, we used the assay for the detection of different non-coding RNA species since they represent promising biomarkers of human disease [39,40,41] but can be challenging to detect [42,43].

CrisprZyme was able to quantify synthetic standards of microRNA-223 (miR-223), aurora kinase A (AURKA) circular RNA (circ-AURKA) and long non-coding RNA LIPCAR (Inc-LIPCAR), all of them showing LODs in the picomolar range (FIG. 4d). To test the assay correlation with a reference standard, we compared the detection of miR-223 by CrisprZyme and rt-qPCR (FIG. 4e). Testing of five different samples containing miR-223 at concentrations between 20 pM and 400 pM showed a high degree of correlation between the two assays, covering cycle threshold (Cₜ) values between 19 and 25 for rt-qPCR reactions.

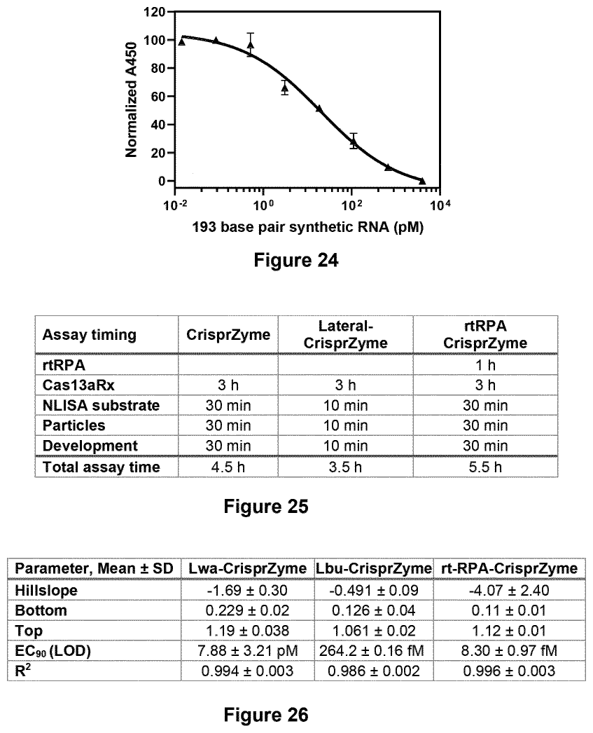

To assess target RNA concentrations amenable for quantitative detection, we tested different Cas13-based assays for their dynamic range. The conventional fluorescence-based Cas13 assay achieved an LOD in the pM range which is consistent with previously reported results (FIG. 24). CrisprZyme showed an LOD of 7.89±3.52 pM, (FIG. 27), and is comparable to other amplification-free strategies that require an electrochemical-powered readout. Importantly, the LOD was similar when using lateral flow as readout or performing the reaction at room temperature underlining its potential for point-of-care applications. Furthermore, a shallow slope of −1.69±0.30 would allow for the use of this technology in a quantitative format. Since various different Cas enzymes exist whose diverse properties can be leveraged for diagnostics, we next tested if CrisprZyme could be combined with LbuCas13a instead of LwaCas13a (FIG. 25 and FIG. 27). While the CRISPR reaction components need to be adapted to meet the requirements of LbuCas13a as previously described, the nanozyme-based readout did not require modifications underscoring its versatility. Importantly, LbuCas13a increased the LOD of the assay to the femtomolar range (264.2±0.20 fM), which further extends the spectrum of potential target analytes.

To further evaluate CrisprZyme's versatility, we investigated the additive combination of CrisprZyme catalysis and rt-RPA amplification including a preamplification step as described in the SHERLOCK technology. We included reverse transcription recombinase polymerase amplification (rt-RPA), followed by T7-based RNA transcription, Cas13 detection and NLISA. This enhanced the sensitivity to an LOD of 8.30±1.06 fM (FIG. 27). However, due to exponential target amplification through rt-RPA, the slope was steeper (−4.07±2.63) limiting its use in quantitative RNA detection and non-communicable diseases. Furthermore, despite the sensitivity enhancement of the NLISA for preamplification-free RNA detection, we discovered that the LOD of the preamplification-based assay is determined by rt-RPA's capability to create copies of the target analyte and inclusion of the NLISA thus does not necessarily reach a lower LOD.

### Example 6—CrisprZyme-Based Detection of ncRNAs in Cell Culture and Human Disease

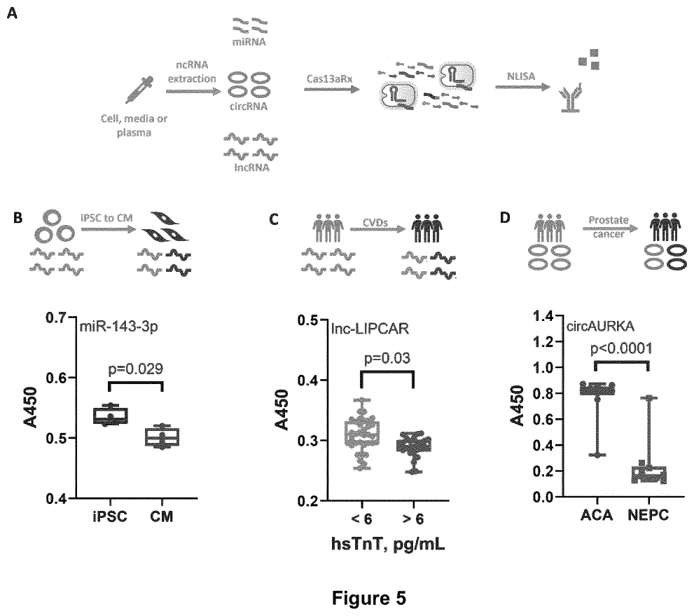

To demonstrate the capability of our newly developed assay, we applied it to sense different ncRNAs (FIG. 5a). We aimed to monitor in vitro differentiation from induced pluripotent stem cells (iPSCs) to cardiomyocytes since cell-type specific expression of miRs can serve as an indicator for cell conversion. We explored the detection of miR-143-3p (FIG. 5b).

CrisprZyme successfully detected upregulation of miR-143-3p with statistical significance in induced cardiomyocytes without preamplification, which was in agreement with previous studies [44].

To prove the usability of CrisprZyme for the diagnosis of cardiovascular events, we tested the blood of 72 patients presenting to emergency rooms with chest pain for expression of Inc-LIPCAR (FIG. 5c). Results showed upregulation of Inc-LIPCAR in patients with levels of high-sensitivity Troponin T (hsTNT) above 6 μg/mL, indicating CrisprZyme's ability to identify patients undergoing a cardiovascular event and correlate it with the levels of hsTnT.

Finally, circ-RNAs can be differentially expressed in human disease and thereby serve as a diagnostic biomarker [45]. Thus, we aimed to differentiate neuroendocrine prostate carcinoma (NEPC) from prostate adenocarcinoma (ACA) by measuring expression of circular Aurora Kinase A RNA (circ-AURKA), which has been identified previously as a marker of NEPC [45](FIG. 5d).

Since circ-AURKA expression is very low in ACA, we aimed for a qualitative assay that detected circ-AURKA in NEPC using rt-RPA and T7 transcription for preamplification of the target region followed by its detection through CrisprZyme. To specifically sense the circular isoform, we positioned the gRNA at the back-splicing junction and used divergent rt-RPA primer for preamplification. CrisprZyme detected circ-AURKA in total RNA isolates from 9 of 10 NEPC biopsies, while only 1 of 10 ACA biopsies were circ-AURKA positive.

### REFERENCES

[1] Pardee, K. et al. Rapid, Low-Cost Detection of Zika Virus Using Programmable Biomolecular Components. Cell 165, 1255-1266 (2016).
[2] Gootenberg, J. S. et a. Nucleic acid detection with CRISPR-Cas13a/C2c2. Science (80-). 356, 438-442 (2017).
[3] Gootenberg, J. S. et a. Multiplexed and portable nucleic acid detection platform with Cas13, Cas12a and Csm6. Science (80-). 360, 439-444 (2018).
[4] Chen, J. S. et al. CRISPR-Cas12a target binding unleashes indiscriminate single-stranded DNase activity. Science 360, 436-439 (2018).
[5] Lee, R. A. et al. Ultrasensitive CRISPR-based diagnostic for field-applicable detection of Plasmodium species in symptomatic and asymptomatic malaria. Proc. Natl. Acad. Sci. U.S.A (2020) doi:10.1073/pnas.2010196117.
[6] Bruch, R. et al. CRISPR/Cas13a-Powered Electrochemical Microfluidic Biosensor for Nucleic Acid Amplification-Free miRNA Diagnostics. Adv. Mater. 31, el 905311 (2019).
[7] Kaminski, M. M. et al. A CRISPR-based assay for the detection of opportunistic infections post-transplantation and for the monitoring of transplant rejection. Nat. Biomed. Eng. 4, 601-609 (2020).
[8] Li, L. et a. HOLMESv2: A CRISPR-Cas12b-Assisted Platform for Nucleic Acid Detection and DNA Methylation Quantitation. ACS Synth. Biol. 8, 2228-2237 (2019).
[9] Dai, Y. et al. Exploring the Trans-Cleavage Activity of CRISPR-Cas12a (cpf1) for the Development of a Universal Electrochemical Biosensor. Angew. Chem. Int. Ed. Engl. 58, 17399-17405 (2019).
[10] Li, S.-Y. et al. CRISPR-Cas12a-assisted nucleic acid detection. Cell Discov. 4, 20 (2018).
[11] Wong, Y.-P., Othman, S., Lau, Y.-L., Radu, S. & Chee, H.-Y. Loop-mediated isothermal amplification (LAMP): a versatile technique for detection of micro-organisms. J. Appl. Microbiol. 124, 626-643 (2018).
[12] Wang, D.-G., Brewster, J. D., Paul, M. & Tomasula, P. M. Two methods for increased specificity and sensitivity in loop-mediated isothermal amplification. Molecules 20, 6048-6059 (2015).
[13] Santiago-Frangos, A. et al. Intrinsic Signal Amplification by Type-III CRISPR-Cas Systems Provides a Sequence-Specific Viral Diagnostic. medRxiv 2020.10.14.20212670 (2020) doi:10.1101/2020.10.14.20212670.
[14] Fozouni, P. et al. Amplification-free detection of SARS-CoV-2 with CRISPR-Cas13a and mobile phone microscopy. Cell 184, 323-333.e9 (2021).
[15] Ding, X. et al. Ultrasensitive and visual detection of SARS-CoV-2 using all-in-one dual CRISPR-Cas12a assay. Nat. Commun. 11, 1-10 (2020).
[16] Loynachan, C. N. et al. Platinum Nanocatalyst Amplification: Redefining the Gold Standard for Lateral Flow Immunoassays with Ultrabroad Dynamic Range. ACS Nano (2018) doi:10.1021/acsnano.7b06229.
[17] Wei, H. & Wang, E. Nanomaterials with enzyme-like characteristics (nanozymes): next-generation artificial enzymes. Chem. Soc. Rev. 42, 6060-93 (2013).
[18] Veitch, N. C. Horseradish peroxidase: A modern view of a classic enzyme. Phytochemistry 65, 249-259 (2004).
[19] Josephy, D., Eling, T. & Mason, R. The Horseradish Peroxidase-catalyzed Oxidation of 3,5,3′,5′-Tetramethylbenzidine. J. Biol. Chem. 257, 3669-3675 (1982).
[20] Gao, L. et al. Intrinsic peroxidase-like activity of ferromagnetic nanoparticles. Nat. Nanotechnol. 2, 577-83 (2007).
[21] Jiang, B. et al. Standardized assays for determining the catalytic activity and kinetics of peroxidase-like nanozymes. Nat. Protoc. 13, 1506-1520 (2018).
[22] Lin, Y., Ren, J. & Qu, X. Nano-gold as artificial enzymes: hidden talents. Adv. Mater. 26, 4200-17 (2014).
[23] Gao, Z., Xu, M., Lu, M., Chen, G. & Tang, D. Urchin-like (gold core)@(platinum shell) nanohybrids: A highly efficient peroxidase-mimetic system for in Situ amplified colorimetric immunoassay. Biosens. Bioelectron. 70, 194-201 (2015).
[24] Xia, X. et al. Pd-Ir Core-Shell Nanocubes: A Type of Highly Efficient and Versatile Peroxidase Mimic. ACS Nano 9, 9994-10004 (2015).
[25] Xi Z, Wei K, Wang Q, Kim M J, Sun S, Fung V, Xia X. Nickel-Platinum Nanoparticles as Peroxidase Mimics with a Record High Catalytic Efficiency. J Am Chem Soc. 2021 Feb. 24; 143(7):2660-2664.
[26] Ye, H. et al. An Enzyme-Free Signal Amplification Technique for Ultrasensitive Colorimetric Assay of Disease Biomarkers. ACS Nano 11, 2052-2059 (2017).
[27]C. N. Loynachan, M. R. Thomas, E. R. Gray, D. A. Richards, J. Kim, B. S. Miller, J. C. Brookes, S. Agarwal, V. Chudasama, R. A. McKendry, M. M. Stevens, Platinum Nanocatalyst Amplification: Redefining the Gold Standard for Lateral Flow Immunoassays with Ultrabroad Dynamic Range, ACS Nano 12(1) (2018) 279-288.
[28] Shmakov, S., Smargon, A., Scott, D. et al. Diversity and evolution of class 2 CRISPR-Cas systems. Nat Rev Microbiol 15, 169-182 (2017).
[29] Zetsche et al.,Cpf1 Is a Single RNA-Guided Endonuclease of a Class 2 CRISPR-Cas System, Cell, Volume 163, Issue 3, 22 Oct. 2015, Pages 759-771 [30] WO 2014/093622
[31] Abudayyeh et al. (2016) “C2c2 is a single-component programmable RNA-guided RNA-targeting CRISPR effector”; Science; DOI: 10.1126/science.aaf5573
[32] Shmakov et al. (2015) “Discovery and Functional Characterization of Diverse Class 2 CRISPR-Cas Systems”, Molecular Cell, DOI: dx.doi.org/10.1016/j.molcel.2015.10.008
[33] Smargon et al. (2017) “Cas13b Is a Type VI-B CRISPR-Associated RNA-Guided RNases Differentially Regulated by Accessory Proteins Csx27 and Csx28,” Molecular Cell. 65, 1-13; dx.doi.org/10.1016/j.molcel.2016.12.023
[34] kazusa.orjp/codon/
[35] Nakamura, Y., et al. “Codon usage tabulated from the international DNA sequence databases: status for the year 2000” Nucl. Acids Res. 28:292 (2000).
[36] Burridge, P. W. et al. Chemically defined generation of human cardiomyocytes. Nat. Methods 11, 855-860 (2014).
[37] Kellner, M. J., Koob, J. G., Gootenberg, J. S., Abudayyeh, O. O. & Zhang, F. SHERLOCK: nucleic acid detection with CRISPR nucleases. Nat. Protoc. 14, 2986-3012 (2019).
[38] Abudayyeh, O. O. et al. RNA targeting with CRISPR-Cas13. Nature 550, 280-284 (2017).
[39] Shi, T., Gao, G. & Cao, Y. Long Noncoding RNAs as Novel Biomarkers Have a Promising Future in Cancer Diagnostics. Dis. Markers 2016, U.S. Pat. No. 9,085,195 (2016).
[40] Beermann, J., Piccoli, M.-T., Viereck, J. & Thum, T. Non-coding RNAs in Development and Disease: Background, Mechanisms, and Therapeutic Approaches. Physiol. Rev. 96, 1297-1325 (2016).
[41] Wang, C. & Jing, Q. Non-coding RNAs as biomarkers for acute myocardial infarction. Acta Pharmacol. Sin. 39, 1110-1119 (2018).
[42] Zhu, C.-S. et al. Avenues Toward microRNA Detection In Vitro: A Review of Technical Advances and Challenges. Comput. Struct. Biotechnol. J. 17, 904-916 (2019).
[43] Dave, V. P. et al. MicroRNA amplification and detection technologies: opportunities and challenges for point of care diagnostics. Lab. Invest. 99, 452-469 (2019).
[44] Garate, X. et al. Identification of the miRNAome of early mesoderm progenitor cells and cardiomyocytes derived from human pluripotent stem cells. Sci. Rep. (2018) doi:10.1038/s41598-018-26156-3.
[45] Chen, S. et al. Widespread and Functional RNA Circularization in Localized Prostate Cancer. Cell (2019) doi:10.1016/j.cell.2019.01.025.

1. A nucleic acid detection system for detection of one or more target nucleic acids comprising:
a) a CRISPR detection composition comprising:
i. a CRISPR effector protein;
ii. one or more guide RNAs (gRNA), each of which is specific for the one or more target nucleic acids; and
iii. a reporter RNA molecule; and
b) a catalytic nanoparticle.

2. The nucleic acid detection system of claim 1, further comprising an RNase inhibitor.

3. The nucleic acid detection system of any preceding claim, wherein the CRISPR effector protein is Cas 13, Cas 12, Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3 or Csf4.

4. The nucleic acid detection system of any preceding claim, wherein the CRISPR effector protein is Cas 13, for example Cas13a or Cas13b.

5. The nucleic acid detection system of any preceding claim, wherein the CRISPR effector protein is Cas 12, for example Cas12a or Cas12b.

6. The nucleic acid detection system of any preceding claim, wherein the one or more target nucleic acids detected is RNA, optionally wherein the RNA is double stranded RNA or single stranded RNA.

7. The nucleic acid detection system of claim 6, wherein the CRISPR effector protein is Cas 13, for example Cas13a or Cas13b.

8. The nucleic acid detection system of any preceding claim, wherein the one or more target nucleic acids detected is DNA, optionally wherein the DNA is double stranded DNA or single stranded DNA.

9. The nucleic acid detection system of claim 8, wherein the CRISPR effector protein is Cas 12, for example Cas12a or Cas12b.

10. The nucleic acid detection system of any preceding claim, wherein the nucleic acid detection system comprises more than one guide RNA, each of which is specific for a different target nucleic acid.

11. The nucleic acid detection system of any preceding claim, wherein the nucleic acid detection system comprises 2, 3, 4, 5, 6, 7, 8, 9, 10, 15, 20, 25, 30, 35, 40, 45, 50, 55, 60, 65, 70, 75, 80, 85, 90, 95 or 100 or more guide RNAs, each of which is specific for a different target nucleic acid.

12. The nucleic acid detection system of any preceding claim, wherein the catalytic nanoparticle comprises one or more compounds selected from the list consisting of: platinum, iron oxide, carbon, gold, palladium, nickel and iridium.

13. The nucleic acid detection system of any preceding claim, wherein the catalytic nanoparticle is a platinum, iron oxide, carbon, gold, palladium, nickel or iridium nanoparticle.

14. The nucleic acid detection system of any preceding claim, wherein the catalytic nanoparticle comprises platinum and gold.

15. The nucleic acid detection system of any preceding claim, wherein the catalytic nanoparticle is a platinum nanoparticle, for example a porous platinum nanoparticle.

16. The nucleic acid detection system of any preceding claim, wherein the average diameter of the catalytic nanoparticle is from about 1 nm to about 10 μm, for example about 1 nm to about 9 μm, about 1 nm to about 8 μm, about 1 nm to about 7 μm, about 1 nm to about 6 μm, about 1 nm to about 5 μm, about 1 nm to about 4 μm, about 1 nm to about 3 μm, about 1 nm to about 2 μm, about 1 nm to about 1 μm, about 1 nm to about 900 nm, about 1 nm to about 800 nm, about 1 nm to about 700 nm, about 1 nm to about 600 nm, about 1 nm to about 500 nm, about 1 nm to about 400 nm, about 1 nm to about 350 nm, about 1 nm to about 300 nm, about 1 nm to about 250 nm, about 1 nm to about 200 nm, about 1 nm to about 150 nm, about 1 nm to about 100 nm, about 1 nm to about 50 nm, about 1 nm to about 40 nm, about 1 nm to about 30 nm, about 1 nm to about 20 nm, about 1 nm to about 10 nm, about 10 nm to about 10 μm, about 10 nm to about 9 μm, about 10 nm to about 8 μm, about 10 nm to about 7 μm, about 10 nm to about 6 μm, about 10 nm to about 5 μm, about 10 nm to about 4 μm, about 10 nm to about 3 μm, about 10 nm to about 2 μm, about 10 nm to about 1 μm, about 10 nm to about 900 nm, about 10 nm to about 800 nm, about 10 nm to about 700 nm, about 10 nm to about 600 nm, about 10 nm to about 500 nm, about 10 nm to about 400 nm, about 10 nm to about 350 nm, about 10 nm to about 300 nm, about 10 nm to about 250 nm, about 10 nm to about 200 nm, about 10 nm to about 150 nm, about 10 nm to about 100 nm, about 10 nm to about 50 nm, about 10 nm to about 40 nm, about 10 nm to about 30 nm, about 10 nm to about 20 nm, about 20 nm to about 10 μm, about 20 nm to about 9 μm, about 20 nm to about 8 μm, about 20 nm to about 7 μm, about 20 nm to about 6 μm, about 20 nm to about 5 μm, about 20 nm to about 4 μm, about 20 nm to about 3 μm, about 20 nm to about 2 μm, about 20 nm to about 1 μm, about 20 nm to about 900 nm, about 20 nm to about 800 nm, about 20 nm to about 700 nm, about 20 nm to about 600 nm, about 20 nm to about 500 nm, about 20 nm to about 400 nm, about 20 nm to about 350 nm, about 20 nm to about 300 nm, about 20 nm to about 250 nm, about 20 nm to about 200 nm, about 20 nm to about 150 nm, about 20 nm to about 100 nm, about 20 nm to about 50 nm, about 20 nm to about 40 nm, about 20 nm to about 30 nm, about 30 nm to about 10 μm, about 30 nm to about 9 μm, about 30 nm to about 8 μm, about 30 nm to about 7 μm, about 30 nm to about 6 μm, about 30 nm to about 5 μm, about 30 nm to about 4 μm, about 30 nm to about 3 μm, about 30 nm to about 2 μm, about 30 nm to about 1 μm, about 30 nm to about 900 nm, about 30 nm to about 800 nm, about 30 nm to about 700 nm, about 30 nm to about 600 nm, about 30 nm to about 500 nm, about 30 nm to about 400 nm, about 30 nm to about 350 nm, about 30 nm to about 300 nm, about 30 nm to about 250 nm, about 30 nm to about 200 nm, about 30 nm to about 150 nm, about 30 nm to about 100 nm, about 30 nm to about 50 nm, about 30 nm to about 40 nm, about 40 nm to about 10 μm, about 40 nm to about 9 μm, about 40 nm to about 8 μm, about 40 nm to about 7 μm, about 40 nm to about 6 μm, about 40 nm to about 5 μm, about 40 nm to about 4 μm, about 40 nm to about 3 μm, about 40 nm to about 2 μm, about 40 nm to about 1 μm, about 40 nm to about 900 nm, about 40 nm to about 800 nm, about 40 nm to about 700 nm, about 40 nm to about 600 nm, about 40 nm to about 500 nm, about 40 nm to about 400 nm, about 40 nm to about 350 nm, about 40 nm to about 300 nm, about 40 nm to about 250 nm, about 40 nm to about 200 nm, about 40 nm to about 150 nm, about 40 nm to about 100 nm, about 40 nm to about 50 nm, about 50 nm to about 10 μm, about 50 nm to about 9 μm, about 50 nm to about 8 μm, about 50 nm to about 7 μm, about 50 nm to about 6 μm, about 50 nm to about 5 μm, about 50 nm to about 4 μm, about 50 nm to about 3 μm, about 50 nm to about 2 μm, about 50 nm to about 1 μm, about 50 nm to about 900 nm, about 50 nm to about 800 nm, about 50 nm to about 700 nm, about 50 nm to about 600 nm, about 50 nm to about 500 nm, about 50 nm to about 400 nm, about 50 nm to about 350 nm, about 50 nm to about 300 nm, about 50 nm to about 250 nm, about 50 nm to about 200 nm, about 50 nm to about 150 nm, about 50 nm to about 100 nm, about 100 nm to about 10 μm, about 100 nm to about 9 μm, about 100 nm to about 8 μm, about 100 nm to about 7 μm, about 100 nm to about 6 μm, about 100 nm to about 5 μm, about 100 nm to about 4 μm, about 100 nm to about 3 μm, about 100 nm to about 2 μm, about 100 nm to about 1 μm, about 100 nm to about 900 nm, about 100 nm to about 800 nm, about 100 nm to about 700 nm, about 100 nm to about 600 nm, about 100 nm to about 500 nm, about 100 nm to about 400 nm, about 100 nm to about 350 nm, about 100 nm to about 300 nm, about 100 nm to about 250 nm, about 100 nm to about 200 nm, about 100 nm to about 150 nm, about 150 nm to about 10 μm, about 150 nm to about 9 μm, about 150 nm to about 8 μm, about 150 nm to about 7 μm, about 150 nm to about 6 μm, about 150 nm to about 5 μm, about 150 nm to about 4 μm, about 150 nm to about 3 μm, about 150 nm to about 2 μm, about 150 nm to about 1 μm, about 150 nm to about 900 nm, about 150 nm to about 800 nm, about 150 nm to about 700 nm, about 150 nm to about 600 nm, about 150 nm to about 500 nm, about 150 nm to about 400 nm, about 150 nm to about 350 nm, about 150 nm to about 300 nm, about 150 nm to about 250 nm, about 150 nm to about 200 nm, about 200 nm to about 10 μm, about 200 nm to about 9 μm, about 200 nm to about 8 μm, about 200 nm to about 7 μm, about 200 nm to about 6 μm, about 200 nm to about 5 μm, about 200 nm to about 4 μm, about 200 nm to about 3 μm, about 200 nm to about 2 μm, about 200 nm to about 1 μm, about 200 nm to about 900 nm, about 200 nm to about 800 nm, about 200 nm to about 700 nm, about 200 nm to about 600 nm, about 200 nm to about 500 nm, about 200 nm to about 400 nm, about 200 nm to about 350 nm, about 200 nm to about 300 nm, about 200 nm to about 250 nm, about 250 nm to about 10 μm, about 250 nm to about 9 μm, about 250 nm to about 8 μm, about 250 nm to about 7 μm, about 250 nm to about 6 μm, about 250 nm to about 5 μm, about 250 nm to about 4 μm, about 250 nm to about 3 μm, about 250 nm to about 2 μm, about 250 nm to about 1 μm, about 250 nm to about 900 nm, about 250 nm to about 800 nm, about 250 nm to about 700 nm, about 250 nm to about 600 nm, about 250 nm to about 500 nm, about 250 nm to about 400 nm, about 250 nm to about 350 nm, about 250 nm to about 300 nm, about 300 nm to about 10 μm, about 300 nm to about 9 μm, about 300 nm to about 8 μm, about 300 nm to about 7 μm, about 300 nm to about 6 μm, about 300 nm to about 5 μm, about 300 nm to about 4 μm, about 300 nm to about 3 μm, about 300 nm to about 2 μm, about 300 nm to about 1 μm, about 300 nm to about 900 nm, about 300 nm to about 800 nm, about 300 nm to about 700 nm, about 300 nm to about 600 nm, about 300 nm to about 500 nm, about 300 nm to about 400 nm, about 300 nm to about 350 nm, about 350 nm to about 10 μm, about 350 nm to about 9 μm, about 350 nm to about 8 μm, about 350 nm to about 7 μm, about 350 nm to about 6 μm, about 350 nm to about 5 μm, about 350 nm to about 4 μm, about 350 nm to about 3 μm, about 350 nm to about 2 μm, about 350 nm to about 1 μm, about 350 nm to about 900 nm, about 350 nm to about 800 nm, about 350 nm to about 700 nm, about 350 nm to about 600 nm, about 350 nm to about 500 nm, about 350 nm to about 400 nm, about 400 nm to about 10 μm, about 400 nm to about 9 μm, about 400 nm to about 8 μm, about 400 nm to about 7 μm, about 400 nm to about 6 μm, about 400 nm to about 5 μm, about 400 nm to about 4 μm, about 400 nm to about 3 μm, about 400 nm to about 2 μm, about 400 nm to about 1 μm, about 400 nm to about 900 nm, about 400 nm to about 800 nm, about 400 nm to about 700 nm, about 400 nm to about 600 nm, about 400 nm to about 500 nm, about 500 nm to about 10 μm, about 500 nm to about 9 μm, about 500 nm to about 8 μm, about 500 nm to about 7 μm, about 500 nm to about 6 μm, about 500 nm to about 5 μm, about 500 nm to about 4 μm, about 500 nm to about 3 μm, about 500 nm to about 2 μm, about 500 nm to about 1 μm, about 500 nm to about 900 nm, about 500 nm to about 800 nm, about 500 nm to about 700 nm, about 500 nm to about 600 nm, about 600 nm to about 10 μm, about 600 nm to about 9 μm, about 600 nm to about 8 μm, about 600 nm to about 7 μm, about 600 nm to about 6 μm, about 600 nm to about 5 μm, about 600 nm to about 4 μm, about 600 nm to about 3 μm, about 600 nm to about 2 μm, about 600 nm to about 1 μm, about 600 nm to about 900 nm, about 600 nm to about 800 nm, about 600 nm to about 700 nm, about 700 nm to about 10 μm, about 700 nm to about 9 μm, about 700 nm to about 8 μm, about 700 nm to about 7 μm, about 700 nm to about 6 μm, about 700 nm to about 5 μm, about 700 nm to about 4 μm, about 700 nm to about 3 μm, about 700 nm to about 2 μm, about 700 nm to about 1 μm, about 700 nm to about 900 nm, about 700 nm to about 800 nm, about 800 nm to about 10 μm, about 800 nm to about 9 μm, about 800 nm to about 8 μm, about 800 nm to about 7 μm, about 800 nm to about 6 μm, about 800 nm to about 5 μm, about 800 nm to about 4 μm, about 800 nm to about 3 μm, about 800 nm to about 2 μm, about 800 nm to about 1 μm, about 800 nm to about 900 nm, about 900 nm to about 10 μm, about 900 nm to about 9 μm, about 900 nm to about 8 μm, about 900 nm to about 7 μm, about 900 nm to about 6 μm, about 900 nm to about 5 μm, about 900 nm to about 4 μm, about 900 nm to about 3 μm, about 900 nm to about 2 μm, about 900 nm to about 1 μm, about 1 μm to about 10 μm, about 1 μm to about 9 μm, about 1 μm to about 8 μm, about 1 μm to about 7 μm, about 1 μm to about 6 μm, about 1 μm to about 5 μm, about 1 μm to about 4 μm, about 1 μm to about 3 μm, about 1 μm to about 2 μm, about 2 μm to about 10 μm, about 2 μm to about 9 μm, about 2 μm to about 8 μm, about 2 μm to about 7 μm, about 2 μm to about 6 μm, about 2 μm to about 5 μm, about 2 μm to about 4 μm, about 2 μm to about 3 μm, about 3 μm to about 10 μm, about 3 μm to about 9 μm, about 3 μm to about 8 μm, about 3 μm to about 7 μm, about 3 μm to about 6 μm, about 3 μm to about 5 μm, about 3 μm to about 4 μm, about 4 μm to about 10 μm, about 4 μm to about 9 μm, about 4 μm to about 8 μm, about 4 μm to about 7 μm, about 4 μm to about 6 μm, about 4 μm to about 5 μm, about 5 μm to about 10 μm, about 5 μm to about 9 μm, about 5 μm to about 8 μm, about 5 μm to about 7 μm, about 5 μm to about 6 μm, about 6 μm to about 10 μm, about 6 μm to about 9 μm, about 6 μm to about 8 μm, about 6 μm to about 7 μm, about 7 μm to about 10 μm, about 7 μm to about 9 μm, about 7 μm to about 8 μm, about 8 μm to about 10 μm, about 8 μm to about 9 μm or about 9 μm to about 10 μm.

17. The nucleic acid detection system of any preceding claim, wherein the average diameter of the catalytic nanoparticle is about 100 nm to 300 nm, for example about 150 nm to about 250 nm.

18. The nucleic acid detection system of any preceding claim, wherein the average diameter of the catalytic nanoparticle is about 188 nm.

19. The nucleic acid detection system of any preceding claim, wherein the catalytic nanoparticle is functionalised with high affinity binders such as biotin-binding proteins, antibodies, nanobodies, affibodies, peptides and/or aptamers.

20. The nucleic acid detection system of any preceding claim, wherein the catalytic nanoparticle is functionalised with streptavidin, neutravidin, antiFAM or antidigoxigenin.

21. The nucleic acid detection system of any preceding claim, wherein the catalytic nanoparticle is functionalised with streptavidin.

22. The nucleic acid detection system of any preceding claim, wherein the concentration of the CRISPR effector protein is from about 1 nM to about 500 nM, for example about 1 nM to about 450 nM, about 1 nM to about 400 nM, about 1 nM to about 350 nM, about 1 nM to about 300 nM, about 1 nM to about 250 nM, about 1 nM to about 200 nM, about 1 nM to about 150 nM, about 1 nM to about 100 nM, about 1 nM to about 90 nM, about 1 nM to about 80 nM, about 1 nM to about 70 nM, about 1 nM to about 60 nM, about 1 nM to about 50 nM, about 1 nM to about 40 nM, about 1 nM to about 30 nM, about 1 nM to about 20 nM, about 1 nM to about 10 nM, about 10 nM to about 500 nM, about 10 nM to about 450 nM, about 10 nM to about 400 nM, about 10 nM to about 350 nM, about 10 nM to about 300 nM, about 10 nM to about 250 nM, about 10 nM to about 200 nM, about 10 nM to about 150 nM, about 10 nM to about 100 nM, about 10 nM to about 90 nM, about 10 nM to about 80 nM, about 10 nM to about 70 nM, about 10 nM to about 60 nM, about 10 nM to about 50 nM, about 10 nM to about 40 nM, about 10 nM to about 30 nM, about 10 nM to about 20 nM, about 20 nM to about 500 nM, about 20 nM to about 450 nM, about 20 nM to about 400 nM, about 20 nM to about 350 nM, about 20 nM to about 300 nM, about 20 nM to about 250 nM, about 20 nM to about 200 nM, about 20 nM to about 150 nM, about 20 nM to about 100 nM, about 20 nM to about 90 nM, about 20 nM to about 80 nM, about 20 nM to about 70 nM, about 20 nM to about 60 nM, about 20 nM to about 50 nM, about 20 nM to about 40 nM, about 20 nM to about 30 nM, about 30 nM to about 500 nM, about 30 nM to about 450 nM, about 30 nM to about 400 nM, about 30 nM to about 350 nM, about 30 nM to about 300 nM, about 30 nM to about 250 nM, about 30 nM to about 200 nM, about 30 nM to about 150 nM, about 30 nM to about 100 nM, about 30 nM to about 90 nM, about 30 nM to about 80 nM, about 30 nM to about 70 nM, about 30 nM to about 60 nM, about 30 nM to about 50 nM, about 30 nM to about 40 nM, about 40 nM to about 500 nM, about 40 nM to about 450 nM, about 40 nM to about 400 nM, about 40 nM to about 350 nM, about 40 nM to about 300 nM, about 40 nM to about 250 nM, about 40 nM to about 200 nM, about 40 nM to about 150 nM, about 40 nM to about 100 nM, about 40 nM to about 90 nM, about 40 nM to about 80 nM, about 40 nM to about 70 nM, about 40 nM to about 60 nM, about 40 nM to about 50 nM, about 50 nM to about 500 nM, about 50 nM to about 450 nM, about 50 nM to about 400 nM, about 50 nM to about 350 nM, about 50 nM to about 300 nM, about 50 nM to about 250 nM, about 50 nM to about 200 nM, about 50 nM to about 150 nM, about 50 nM to about 100 nM, about 50 nM to about 90 nM, about 50 nM to about 80 nM, about 50 nM to about 70 nM, about 50 nM to about 60 nM, about 60 nM to about 500 nM, about 60 nM to about 450 nM, about 60 nM to about 400 nM, about 60 nM to about 350 nM, about 60 nM to about 300 nM, about 60 nM to about 250 nM, about 60 nM to about 200 nM, about 60 nM to about 150 nM, about 60 nM to about 100 nM, about 60 nM to about 90 nM, about 60 nM to about 80 nM, about 60 nM to about 70 nM, about 70 nM to about 500 nM, about 70 nM to about 450 nM, about 70 nM to about 400 nM, about 70 nM to about 350 nM, about 70 nM to about 300 nM, about 70 nM to about 250 nM, about 70 nM to about 200 nM, about 70 nM to about 150 nM, about 70 nM to about 100 nM, about 70 nM to about 90 nM, about 70 nM to about 80 nM, about 80 nM to about 500 nM, about 80 nM to about 450 nM, about 80 nM to about 400 nM, about 80 nM to about 350 nM, about 80 nM to about 300 nM, about 80 nM to about 250 nM, about 80 nM to about 200 nM, about 80 nM to about 150 nM, about 80 nM to about 100 nM, about 80 nM to about 90 nM, about 90 nM to about 500 nM, about 90 nM to about 450 nM, about 90 nM to about 400 nM, about 90 nM to about 350 nM, about 90 nM to about 300 nM, about 90 nM to about 250 nM, about 90 nM to about 200 nM, about 90 nM to about 150 nM, about 90 nM to about 100 nM, about 100 nM to about 500 nM, about 100 nM to about 450 nM, about 100 nM to about 400 nM, about 100 nM to about 350 nM, about 100 nM to about 300 nM, about 100 nM to about 250 nM, about 100 nM to about 200 nM, about 100 nM to about 150 nM, about 150 nM to about 500 nM, about 150 nM to about 450 nM, about 150 nM to about 400 nM, about 150 nM to about 350 nM, about 150 nM to about 300 nM, about 150 nM to about 250 nM, about 150 nM to about 200 nM, about 200 nM to about 500 nM, about 200 nM to about 450 nM, about 200 nM to about 400 nM, about 200 nM to about 350 nM, about 200 nM to about 300 nM, about 200 nM to about 250 nM, about 250 nM to about 500 nM, about 250 nM to about 450 nM, about 250 nM to about 400 nM, about 250 nM to about 350 nM, about 250 nM to about 300 nM, about 300 nM to about 500 nM, about 300 nM to about 450 nM, about 300 nM to about 400 nM, about 300 nM to about 350 nM, about 350 nM to about 500 nM, about 350 nM to about 450 nM, about 350 nM to about 400 nM, about 400 nM to about 500 nM, about 400 nM to about 450 nM or about 450 nM to about 500 nM.

23. The nucleic acid detection system of any preceding claim, wherein the concentration of the CRISPR effector protein is from about 100 nM to 500 nM, for example about 200 nM to about 400 nM.

24. The nucleic acid detection system of any preceding claim, wherein the concentration of the CRISPR effector protein is about 300 nM.

25. The nucleic acid detection system of any preceding claim, wherein the CRISPR effector protein is derived from a bacterial strain selected from the list consisting of: Leptotrichia wadei (F0279), Leptotrichia shahii, Lachnospiraceae bacterium (MA2020), Lachnospiraceae bacterium (NK4A179), Clostridium aminophilum (DSM 10710), Carnobacterium gallinarum (DSM 4847), Paludibacter propionicigenes (WB4), Listeria weihenstephanensis (FSL R9-0317), Listeriaceae bacterium (FSL M6-0635), Listeria newyorkensis (FSL M6-0635), Rhodobacter capsulatus (SB 1003), Rhodobacter capsulatus (R121), Rhodobacter capsulatus (DE442), Leptotrichia wadei (Lw2), Leptotrichia buccalis (Lbu) and Listeria seeligeri.

26. The nucleic acid detection system of any preceding claim, wherein the CRISPR effector protein is derived from Leptotrichia wadei.

27. The nucleic acid detection system of any preceding claim, wherein the CRISPR effector protein is a Cas13 protein, such as a Cas13a or Cas13b protein, derived from a bacterial strain selected from the list consisting of: Leptotrichia wadei (F0279), Leptotrichia shahii, Lachnospiraceae bacterium (MA2020), Lachnospiraceae bacterium (NK4A179), Clostridium aminophilum (DSM 10710), Carnobacterium gallinarum (DSM 4847), Paludibacter propionicigenes (WB4), Listeria weihenstephanensis (FSL R9-0317), Listeriaceae bacterium (FSL M6-0635), Listeria newyorkensis (FSL M6-0635), Rhodobacter capsulatus (SB 1003), Rhodobacter capsulatus (R121), Rhodobacter capsulatus (DE442), Leptotrichia wadei (Lw2), Leptotrichia buccalis (Lbu) and Listeria seeligeri.

28. The nucleic acid detection system of any preceding claim, wherein the CRISPR effector protein is a Cas13 protein, such as a Cas13a or Cas13b protein, derived from Leptotrichia wadei.

29. The nucleic acid detection system of any preceding claim, wherein the CRISPR effector protein is a Cas12 protein, such as a Cas12a or Cas12b protein, derived from a bacterial strain selected from the list consisting of: Leptotrichia wadei (F0279), Leptotrichia shahii, Lachnospiraceae bacterium (MA2020), Lachnospiraceae bacterium (NK4A179), Clostridium aminophilum (DSM 10710), Carnobacterium gallinarum (DSM 4847), Paludibacter propionicigenes (WB4), Listeria weihenstephanensis (FSL R9-0317), Listeriaceae bacterium (FSL M6-0635), Listeria newyorkensis (FSL M6-0635), Rhodobacter capsulatus (SB 1003), Rhodobacter capsulatus (R121), Rhodobacter capsulatus (DE442), Leptotrichia wadei (Lw2), Leptotrichia buccalis (Lbu) and Listeria seeligeri.

30. The nucleic acid detection system of any preceding claim, wherein the CRISPR effector protein is a Cas12 protein, such as a Cas12a or Cas12b protein, derived from Leptotrichia wadei.

31. The nucleic acid detection system of any preceding claim, wherein the concentration of the reporter RNA molecule is from about 0.1 nM to about 500 nM, for example about 0.1 nM to about 400 nM, about 0.1 nM to about 300 nM, about 0.1 nM to about 200 nM, about 0.1 nM to about 100 nM, about 0.1 nM to about 90 nM, about 0.1 nM to about 80 nM, about 0.1 nM to about 70 nM, about 0.1 nM to about 60 nM, about 0.1 nM to about 50 nM, about 0.1 nM to about 40 nM, about 0.1 nM to about 30 nM, about 0.1 nM to about 20 nM, about 0.1 nM to about 10 nM, about 0.1 nM to about 9 nM, about 0.1 nM to about 8 nM, about 0.1 nM to about 7 nM, about 0.1 nM to about 6 nM, about 0.1 nM to about 5 nM, about 0.1 nM to about 4 nM, about 0.1 nM to about 3 nM, about 0.1 nM to about 2 nM, about 0.1 nM to about 1 nM, about 0.1 nM to about 0.9 nM, about 0.1 nM to about 0.8 nM, about 0.1 nM to about 0.7 nM, about 0.1 nM to about 0.6 nM, about 0.1 nM to about 0.5 nM, about 0.1 nM to about 0.4 nM, about 0.1 nM to about 0.3 nM, about 0.1 nM to about 0.2 nM, about 0.2 nM to about 500 nM, about 0.2 nM to about 400 nM, about 0.2 nM to about 300 nM, about 0.2 nM to about 200 nM, about 0.2 nM to about 100 nM, about 0.2 nM to about 90 nM, about 0.2 nM to about 80 nM, about 0.2 nM to about 70 nM, about 0.2 nM to about 60 nM, about 0.2 nM to about 50 nM, about 0.2 nM to about 40 nM, about 0.2 nM to about 30 nM, about 0.2 nM to about 20 nM, about 0.2 nM to about 10 nM, about 0.2 nM to about 9 nM, about 0.2 nM to about 8 nM, about 0.2 nM to about 7 nM, about 0.2 nM to about 6 nM, about 0.2 nM to about 5 nM, about 0.2 nM to about 4 nM, about 0.2 nM to about 3 nM, about 0.2 nM to about 2 nM, about 0.2 nM to about 1 nM, about 0.2 nM to about 0.9 nM, about 0.2 nM to about 0.8 nM, about 0.2 nM to about 0.7 nM, about 0.2 nM to about 0.6 nM, about 0.2 nM to about 0.5 nM, about 0.2 nM to about 0.4 nM, about 0.2 nM to about 0.3 nM, about 0.3 nM to about 500 nM, about 0.3 nM to about 400 nM, about 0.3 nM to about 300 nM, about 0.3 nM to about 200 nM, about 0.3 nM to about 100 nM, about 0.3 nM to about 90 nM, about 0.3 nM to about 80 nM, about 0.3 nM to about 70 nM, about 0.3 nM to about 60 nM, about 0.3 nM to about 50 nM, about 0.3 nM to about 40 nM, about 0.3 nM to about 30 nM, about 0.3 nM to about 20 nM, about 0.3 nM to about 10 nM, about 0.3 nM to about 9 nM, about 0.3 nM to about 8 nM, about 0.3 nM to about 7 nM, about 0.3 nM to about 6 nM, about 0.3 nM to about 5 nM, about 0.3 nM to about 4 nM, about 0.3 nM to about 3 nM, about 0.3 nM to about 2 nM, about 0.3 nM to about 1 nM, about 0.3 nM to about 0.9 nM, about 0.3 nM to about 0.8 nM, about 0.3 nM to about 0.7 nM, about 0.3 nM to about 0.6 nM, about 0.3 nM to about 0.5 nM, about 0.3 nM to about 0.4 nM, about 0.4 nM to about 500 nM, about 0.4 nM to about 400 nM, about 0.4 nM to about 300 nM, about 0.4 nM to about 200 nM, about 0.4 nM to about 100 nM, about 0.4 nM to about 90 nM, about 0.4 nM to about 80 nM, about 0.4 nM to about 70 nM, about 0.4 nM to about 60 nM, about 0.4 nM to about 50 nM, about 0.4 nM to about 40 nM, about 0.4 nM to about 30 nM, about 0.4 nM to about 20 nM, about 0.4 nM to about 10 nM, about 0.4 nM to about 9 nM, about 0.4 nM to about 8 nM, about 0.4 nM to about 7 nM, about 0.4 nM to about 6 nM, about 0.4 nM to about 5 nM, about 0.4 nM to about 4 nM, about 0.4 nM to about 3 nM, about 0.4 nM to about 2 nM, about 0.4 nM to about 1 nM, about 0.4 nM to about 0.9 nM, about 0.4 nM to about 0.8 nM, about 0.4 nM to about 0.7 nM, about 0.4 nM to about 0.6 nM, about 0.4 nM to about 0.5 nM, about 0.5 nM to about 500 nM, about 0.5 nM to about 400 nM, about 0.5 nM to about 300 nM, about 0.5 nM to about 200 nM, about 0.5 nM to about 100 nM, about 0.5 nM to about 90 nM, about 0.5 nM to about 80 nM, about 0.5 nM to about 70 nM, about 0.5 nM to about 60 nM, about 0.5 nM to about 50 nM, about 0.5 nM to about 40 nM, about 0.5 nM to about 30 nM, about 0.5 nM to about 20 nM, about 0.5 nM to about 10 nM, about 0.5 nM to about 9 nM, about 0.5 nM to about 8 nM, about 0.5 nM to about 7 nM, about 0.5 nM to about 6 nM, about 0.5 nM to about 5 nM, about 0.5 nM to about 4 nM, about 0.5 nM to about 3 nM, about 0.5 nM to about 2 nM, about 0.5 nM to about 1 nM, about 0.5 nM to about 0.9 nM, about 0.5 nM to about 0.8 nM, about 0.5 nM to about 0.7 nM, about 0.5 nM to about 0.6 nM, about 0.6 nM to about 500 nM, about 0.6 nM to about 400 nM, about 0.6 nM to about 300 nM, about 0.6 nM to about 200 nM, about 0.6 nM to about 100 nM, about 0.6 nM to about 90 nM, about 0.6 nM to about 80 nM, about 0.6 nM to about 70 nM, about 0.6 nM to about 60 nM, about 0.6 nM to about 50 nM, about 0.6 nM to about 40 nM, about 0.6 nM to about 30 nM, about 0.6 nM to about 20 nM, about 0.6 nM to about 10 nM, about 0.6 nM to about 9 nM, about 0.6 nM to about 8 nM, about 0.6 nM to about 7 nM, about 0.6 nM to about 6 nM, about 0.6 nM to about 5 nM, about 0.6 nM to about 4 nM, about 0.6 nM to about 3 nM, about 0.6 nM to about 2 nM, about 0.6 nM to about 1 nM, about 0.6 nM to about 0.9 nM, about 0.6 nM to about 0.8 nM, about 0.6 nM to about 0.7 nM, about 0.7 nM to about 500 nM, about 0.7 nM to about 400 nM, about 0.7 nM to about 300 nM, about 0.7 nM to about 200 nM, about 0.7 nM to about 100 nM, about 0.7 nM to about 90 nM, about 0.7 nM to about 80 nM, about 0.7 nM to about 70 nM, about 0.7 nM to about 60 nM, about 0.7 nM to about 50 nM, about 0.7 nM to about 40 nM, about 0.7 nM to about 30 nM, about 0.7 nM to about 20 nM, about 0.7 nM to about 10 nM, about 0.7 nM to about 9 nM, about 0.7 nM to about 8 nM, about 0.7 nM to about 7 nM, about 0.7 nM to about 6 nM, about 0.7 nM to about 5 nM, about 0.7 nM to about 4 nM, about 0.7 nM to about 3 nM, about 0.7 nM to about 2 nM, about 0.7 nM to about 1 nM, about 0.7 nM to about 0.9 nM, about 0.7 nM to about 0.8 nM, about 0.8 nM to about 500 nM, about 0.8 nM to about 400 nM, about 0.8 nM to about 300 nM, about 0.8 nM to about 200 nM, about 0.8 nM to about 100 nM, about 0.8 nM to about 90 nM, about 0.8 nM to about 80 nM, about 0.8 nM to about 70 nM, about 0.8 nM to about 60 nM, about 0.8 nM to about 50 nM, about 0.8 nM to about 40 nM, about 0.8 nM to about 30 nM, about 0.8 nM to about 20 nM, about 0.8 nM to about 10 nM, about 0.8 nM to about 9 nM, about 0.8 nM to about 8 nM, about 0.8 nM to about 7 nM, about 0.8 nM to about 6 nM, about 0.8 nM to about 5 nM, about 0.8 nM to about 4 nM, about 0.8 nM to about 3 nM, about 0.8 nM to about 2 nM, about 0.8 nM to about 1 nM, about 0.8 nM to about 0.9 nM, about 0.9 nM to about 500 nM, about 0.9 nM to about 400 nM, about 0.9 nM to about 300 nM, about 0.9 nM to about 200 nM, about 0.9 nM to about 100 nM, about 0.9 nM to about 90 nM, about 0.9 nM to about 80 nM, about 0.9 nM to about 70 nM, about 0.9 nM to about 60 nM, about 0.9 nM to about 50 nM, about 0.9 nM to about 40 nM, about 0.9 nM to about 30 nM, about 0.9 nM to about 20 nM, about 0.9 nM to about 10 nM, about 0.9 nM to about 9 nM, about 0.9 nM to about 8 nM, about 0.9 nM to about 7 nM, about 0.9 nM to about 6 nM, about 0.9 nM to about 5 nM, about 0.9 nM to about 4 nM, about 0.9 nM to about 3 nM, about 0.9 nM to about 2 nM, about 0.9 nM to about 1 nM, about 1 nM to about 500 nM, about 1 nM to about 400 nM, about 1 nM to about 300 nM, about 1 nM to about 200 nM, about 1 nM to about 100 nM, about 1 nM to about 90 nM, about 1 nM to about 80 nM, about 1 nM to about 70 nM, about 1 nM to about 60 nM, about 1 nM to about 50 nM, about 1 nM to about 40 nM, about 1 nM to about 30 nM, about 1 nM to about 20 nM, about 1 nM to about 10 nM, about 1 nM to about 9 nM, about 1 nM to about 8 nM, about 1 nM to about 7 nM, about 1 nM to about 6 nM, about 1 nM to about 5 nM, about 1 nM to about 4 nM, about 1 nM to about 3 nM, about 1 nM to about 2 nM, about 2 nM to about 500 nM, about 2 nM to about 400 nM, about 2 nM to about 300 nM, about 2 nM to about 200 nM, about 2 nM to about 100 nM, about 2 nM to about 90 nM, about 2 nM to about 80 nM, about 2 nM to about 70 nM, about 2 nM to about 60 nM, about 2 nM to about 50 nM, about 2 nM to about 40 nM, about 2 nM to about 30 nM, about 2 nM to about 20 nM, about 2 nM to about 10 nM, about 2 nM to about 9 nM, about 2 nM to about 8 nM, about 2 nM to about 7 nM, about 2 nM to about 6 nM, about 2 nM to about 5 nM, about 2 nM to about 4 nM, about 2 nM to about 3 nM, about 3 nM to about 500 nM, about 3 nM to about 400 nM, about 3 nM to about 300 nM, about 3 nM to about 200 nM, about 3 nM to about 100 nM, about 3 nM to about 90 nM, about 3 nM to about 80 nM, about 3 nM to about 70 nM, about 3 nM to about 60 nM, about 3 nM to about 50 nM, about 3 nM to about 40 nM, about 3 nM to about 30 nM, about 3 nM to about 20 nM, about 3 nM to about 10 nM, about 3 nM to about 9 nM, about 3 nM to about 8 nM, about 3 nM to about 7 nM, about 3 nM to about 6 nM, about 3 nM to about 5 nM, about 3 nM to about 4 nM, about 4 nM to about 500 nM, about 4 nM to about 400 nM, about 4 nM to about 300 nM, about 4 nM to about 200 nM, about 4 nM to about 100 nM, about 4 nM to about 90 nM, about 4 nM to about 80 nM, about 4 nM to about 70 nM, about 4 nM to about 60 nM, about 4 nM to about 50 nM, about 4 nM to about 40 nM, about 4 nM to about 30 nM, about 4 nM to about 20 nM, about 4 nM to about 10 nM, about 4 nM to about 9 nM, about 4 nM to about 8 nM, about 4 nM to about 7 nM, about 4 nM to about 6 nM, about 4 nM to about 5 nM, about 5 nM to about 500 nM, about 5 nM to about 400 nM, about 5 nM to about 300 nM, about 5 nM to about 200 nM, about 5 nM to about 100 nM, about 5 nM to about 90 nM, about 5 nM to about 80 nM, about 5 nM to about 70 nM, about 5 nM to about 60 nM, about 5 nM to about 50 nM, about 5 nM to about 40 nM, about 5 nM to about 30 nM, about 5 nM to about 20 nM, about 5 nM to about 10 nM, about 5 nM to about 9 nM, about 5 nM to about 8 nM, about 5 nM to about 7 nM, about 5 nM to about 6 nM, about 6 nM to about 500 nM, about 6 nM to about 400 nM, about 6 nM to about 300 nM, about 6 nM to about 200 nM, about 6 nM to about 100 nM, about 6 nM to about 90 nM, about 6 nM to about 80 nM, about 6 nM to about 70 nM, about 6 nM to about 60 nM, about 6 nM to about 50 nM, about 6 nM to about 40 nM, about 6 nM to about 30 nM, about 6 nM to about 20 nM, about 6 nM to about 10 nM, about 6 nM to about 9 nM, about 6 nM to about 8 nM, about 6 nM to about 7 nM, about 7 nM to about 500 nM, about 7 nM to about 400 nM, about 7 nM to about 300 nM, about 7 nM to about 200 nM, about 7 nM to about 100 nM, about 7 nM to about 90 nM, about 7 nM to about 80 nM, about 7 nM to about 70 nM, about 7 nM to about 60 nM, about 7 nM to about 50 nM, about 7 nM to about 40 nM, about 7 nM to about 30 nM, about 7 nM to about 20 nM, about 7 nM to about 10 nM, about 7 nM to about 9 nM, about 7 nM to about 8 nM, about 8 nM to about 500 nM, about 8 nM to about 400 nM, about 8 nM to about 300 nM, about 8 nM to about 200 nM, about 8 nM to about 100 nM, about 8 nM to about 90 nM, about 8 nM to about 80 nM, about 8 nM to about 70 nM, about 8 nM to about 60 nM, about 8 nM to about 50 nM, about 8 nM to about 40 nM, about 8 nM to about 30 nM, about 8 nM to about 20 nM, about 8 nM to about 10 nM, about 8 nM to about 9 nM, about 9 nM to about 500 nM, about 9 nM to about 400 nM, about 9 nM to about 300 nM, about 9 nM to about 200 nM, about 9 nM to about 100 nM, about 9 nM to about 90 nM, about 9 nM to about 80 nM, about 9 nM to about 70 nM, about 9 nM to about 60 nM, about 9 nM to about 50 nM, about 9 nM to about 40 nM, about 9 nM to about 30 nM, about 9 nM to about 20 nM, about 9 nM to about 10 nM, about 10 nM to about 500 nM, about 10 nM to about 400 nM, about 10 nM to about 300 nM, about 10 nM to about 200 nM, about 10 nM to about 100 nM, about 10 nM to about 90 nM, about 10 nM to about 80 nM, about 10 nM to about 70 nM, about 10 nM to about 60 nM, about 10 nM to about 50 nM, about 10 nM to about 40 nM, about 10 nM to about 30 nM, about 10 nM to about 20 nM, about 20 nM to about 500 nM, about 20 nM to about 400 nM, about 20 nM to about 300 nM, about 20 nM to about 200 nM, about 20 nM to about 100 nM, about 20 nM to about 90 nM, about 20 nM to about 80 nM, about 20 nM to about 70 nM, about 20 nM to about 60 nM, about 20 nM to about 50 nM, about 20 nM to about 40 nM, about 20 nM to about 30 nM, about 30 nM to about 500 nM, about 30 nM to about 400 nM, about 30 nM to about 300 nM, about 30 nM to about 200 nM, about 30 nM to about 100 nM, about 30 nM to about 90 nM, about 30 nM to about 80 nM, about 30 nM to about 70 nM, about 30 nM to about 60 nM, about 30 nM to about 50 nM, about 30 nM to about 40 nM, about 40 nM to about 500 nM, about 40 nM to about 400 nM, about 40 nM to about 300 nM, about 40 nM to about 200 nM, about 40 nM to about 100 nM, about 40 nM to about 90 nM, about 40 nM to about 80 nM, about 40 nM to about 70 nM, about 40 nM to about 60 nM, about 40 nM to about 50 nM, about 50 nM to about 500 nM, about 50 nM to about 400 nM, about 50 nM to about 300 nM, about 50 nM to about 200 nM, about 50 nM to about 100 nM, about 50 nM to about 90 nM, about 50 nM to about 80 nM, about 50 nM to about 70 nM, about 50 nM to about 60 nM, about 60 nM to about 500 nM, about 60 nM to about 400 nM, about 60 nM to about 300 nM, about 60 nM to about 200 nM, about 60 nM to about 100 nM, about 60 nM to about 90 nM, about 60 nM to about 80 nM, about 60 nM to about 70 nM, about 70 nM to about 500 nM, about 70 nM to about 400 nM, about 70 nM to about 300 nM, about 70 nM to about 200 nM, about 70 nM to about 100 nM, about 70 nM to about 90 nM, about 70 nM to about 80 nM, about 80 nM to about 500 nM, about 80 nM to about 400 nM, about 80 nM to about 300 nM, about 80 nM to about 200 nM, about 80 nM to about 100 nM, about 80 nM to about 90 nM, about 90 nM to about 500 nM, about 90 nM to about 400 nM, about 90 nM to about 300 nM, about 90 nM to about 200 nM, about 90 nM to about 100 nM, about 100 nM to about 500 nM, about 100 nM to about 400 nM, about 100 nM to about 300 nM, about 100 nM to about 200 nM, about 200 nM to about 500 nM, about 200 nM to about 400 nM, about 200 nM to about 300 nM, about 300 nM to about 500 nM, about 300 nM to about 400 nM or about 400 nM to about 500 nM.

32. The nucleic acid detection system of any preceding claim, wherein the concentration of the reporter RNA is from about 0.1 nM to 2 nM, for example about 0.5 nM to about 1 nM.

33. The nucleic acid detection system of any preceding claim, wherein the concentration of the reporter RNA is about 0.75 nM.

34. The nucleic acid detection system of any preceding claim, wherein the concentration of one or more of the components is the concentration of said component in the assay mastermix.

35. The nucleic acid detection system of any preceding claim, wherein the system is for use at a temperature of about 5° C. to about 50° C., for example about 5° C. to about 45° C., about 5° C. to about 40° C., about 5° C. to about 35° C., about 5° C. to about 30° C., about 5° C. to about 25° C., about 5° C. to about 20° C., about 5° C. to about 15° C., about 5° C. to about 10° C., about 10° C. to about 50° C., about 10° C. to about 45° C., about 10° C. to about 40° C., about 10° C. to about 35° C., about 10° C. to about 30° C., about 10° C. to about 25° C., about 10° C. to about 20° C., about 10° C. to about 15° C., about 15° C. to about 50° C., about 15° C. to about 45° C., about 15° C. to about 40° C., about 15° C. to about 35° C., about 15° C. to about 30° C., about 15° C. to about 25° C., about 15° C. to about 20° C., about 20° C. to about 50° C., about 20° C. to about 45° C., about 20° C. to about 40° C., about 20° C. to about 35° C., about 20° C. to about 30° C., about 20° C. to about 25° C., about 25° C. to about 50° C., about 25° C. to about 45° C., about 25° C. to about 40° C., about 25° C. to about 35° C., about 25° C. to about 30° C., about 30° C. to about 50° C., about 30° C. to about 45° C., about 30° C. to about 40° C., about 30° C. to about 35° C., about 35° C. to about 50° C., about 35° C. to about 45° C., about 35° C. to about 40° C., about 40° C. to about 50° C., about 40° C. to about 45° C. or about 45° C. to about 50° C.

36. The nucleic acid detection system of any preceding claim, wherein the system is for use at a temperature of about 15° C. to about 37° C., for example about 20° C. or about 25° C.

37. The nucleic acid detection system of any preceding claim, wherein the system is for use in a multiwell plate format, glass slides, nitrocellulose or microfluidic device.

38. The nucleic acid detection system of any preceding claim, wherein the system is for use in a 96-well or 384-well plate.

39. The nucleic acid detection system of any preceding claim, wherein the target nucleic acid is a messenger RNA (mRNA), non-coding RNA (ncRNA), microRNA (miRNA), a long non-coding RNA (IncRNA) or a circular RNA (circRNA).

40. The nucleic acid detection system of any preceding claim, wherein the target nucleic acid is a DNA molecule, such as genomic DNA (gDNA) or complementary DNA (cDNA).

41. The nucleic acid detection system of any preceding claim, wherein the target nucleic acid is from a viral, bacterial or human source.

42. The nucleic acid detection system of any preceding claim, wherein the target nucleic acid is from SARS-CoV-2.

43. The nucleic acid detection system of any preceding claim, wherein the target nucleic acid is a cardiovascular event biomarker or a prostate cancer biomarker.

44. A reporter RNA molecule for a CRISPR detection assay comprising at least two functional handles, for example two, three, four, five, six, seven, eight or more functional handles.

45. The reporter RNA molecule of claim 44, comprising at least three functional handles, for example three, four, five, six, seven, eight or more functional handles.

46. The reporter RNA molecule of claim 45, wherein the at least three functional handles comprise digoxigenin, biotin and FAM.

47. The reporter RNA molecule of any one of claims 44 to 46 comprising a first functional handle in the 5′-end.

48. The reporter RNA molecule of any one of claims 44 to 47 comprising a second functional handle in the 3′-end, which is different to the first functional handle and has orthogonal binding to at the first functional handle of the 5′-end.

49. The reporter RNA molecule of any one of claims 44 to 48 comprising a third functional handle with orthogonal binding to the first and second functional handles linked to either the 5′-end or 3′-end handle.

50. The reporter RNA molecule of claim 49, wherein the third functional handle is attached to the reporter RNA via a covalent bond.

51. The reporter RNA molecule of claim 50, wherein the covalent bond contains a spacer arm that can be any polymeric sequence comprising peptides, RNA or organic polymers.

52. The reporter RNA molecule of claim 51, wherein the spacer arm comprises a PEG sequence of up to 10 KDa, for example a spacer arm comprising triethyleneglycol.

53. The reporter RNA molecule of any one of claims 44 to 52 comprising an RNA sequence of up to 50 nucleobases to be cleaved by an effector protein, such up to 10, 20, 30 or 40 nucleobases.

54. The reporter RNA molecule of any one of claims 44 to 53 comprising an RNA sequence of 14 nucleobases to be cleaved by an effector protein.

55. The reporter RNA molecule of any one of claims 44 to 54 comprising an RNA sequence of 6 nucleobases to be cleaved by an effector protein.

56. A method of detecting a nucleic acid of interest comprising:
a. contacting a sample with a nucleic acid detection system for detection of one or more target nucleic acids comprising:
i. a CRISPR detection composition comprising:
I. a CRISPR effector protein;
II. one or more guide RNAs (gRNA), each of which is specific for the one or more target nucleic acids; and
III. a reporter RNA molecule; and
ii. a catalytic nanoparticle;
b. adding a chromogenic substrate; and
c. detecting the presence of the nucleic acid of interest based on the catalysis of the chromogenic substrate.

57. A method of detecting a nucleic acid of interest comprising:
a. contacting a sample with a nucleic acid detection system of any one of claims 1 to 43;
b. adding a chromogenic substrate; and
c. detecting the presence of the nucleic acid of interest based on the catalysis of the chromogenic substrate.

58. The method of detecting a nucleic acid of interest of claim 56 or claim 57, wherein the presence of the nucleic acid of interest is used to diagnose a disease.

59. The method of detecting a nucleic acid of interest of any one of claims 56 to 58, wherein the presence of the nucleic acid of interest is used to diagnose a disease selected from the list consisting of respiratory diseases, HIV, tuberculosis, SARS-CoV-2, cardiovascular diseases, cancer or Alzheimer's disease.

60. A method of diagnosing a disease in a patient comprising detecting a nucleic acid of interest in a biological sample derived from a patient comprising:
a. contacting a sample with a nucleic acid detection system for detection of one or more target nucleic acids comprising:
i. a CRISPR detection composition comprising:
I. a CRISPR effector protein;
II. one or more guide RNAs (gRNA), each of which is specific for the one or more target nucleic acids; and
III. a reporter RNA molecule; and
ii. a catalytic nanoparticle;
b. adding a chromogenic substrate;
c. detecting the presence of the nucleic acid of interest based on the catalysis of the chromogenic substrate;
d. diagnosing the disease based on the presence of the nucleic acid of interest.

61. A method of diagnosing a disease in a patient comprising detecting a nucleic acid of interest in a biological sample derived from a patient comprising:
a. contacting a sample with a nucleic acid detection system of any one of claims 1 to 43;
b. adding a chromogenic substrate; and
c. detecting the presence of the nucleic acid of interest based on the catalysis of the chromogenic substrate;
d. diagnosing the disease based on the presence of the nucleic acid of interest.

62. The method according to claim 61 wherein the biological sample is a cell extract, a blood sample, for example a whole blood sample or a blood fraction such as blood serum, a tissue biopsy, amniotic fluid; aqueous humour; bile; blood plasma; breast milk; cerebrospinal fluid (CSF), endolymph, extracellular fluid, exudate, gastric acid, hemolymph, interstitial fluid, lymph, mucus, pericardial fluid, peritoneal fluid, perspiration (sweat), phlegm, pus, saliva, semen, synovial fluid, tears, urine, vaginal fluids, vomit, sputum, other biofluid or swab sample.

63. The method according to any one of claims 61 to 62, wherein the biological sample is a cell extract, a blood sample or a tissue biopsy.

64. The method according to any one of claims 56 to 63 wherein detecting the presence of a nucleic acid of interest based on the catalysis of a chromogenic molecule comprises detecting the presence of a nucleic acid of interest based on catalysis of a chromogenic molecule.

65. The method according to any one of claims 56 to 63 wherein detecting the presence of a nucleic acid of interest based on the catalysis of a chromogenic molecule comprises detecting the presence of a nucleic acid of interest based on the absence of catalysis of a chromogenic molecule.

66. The method according to any one of claims 56 to 65, wherein the chromogenic substrate is selected from the list consisting of: TMB (3,3′,5,5′-tetramethylbenzidine), CN (4-chloro-1-naphthol) and DAB (3,3′-diaminobenzidine tetrahydrochloride), PNPP (p-Nitrophenyl Phosphate), ABTS (2,2′-Azinobis [3-ethylbenzothiazoline-6-sulfonic acid]), OPD (o-phenylenediamine dihydrochloride) and ONGP (ortho-Nitrophenyl-β-galactoside).

67. The method according to any one of claims 56 to 66, wherein the chromogenic substrate is 3,3′,5,5′-tetramethylbenzidine (TMB).

68. The method according to any one of claims 56 to 67, wherein the concentration of the chromogenic substrate is from about 0.001% (w/v) to about 0.1% (w/v), for example about 0.001% (w/v) to about 0.09% (w/v), about 0.001% (w/v) to about 0.08% (w/v), about 0.001% (w/v) to about 0.07% (w/v), about 0.001% (w/v) to about 0.06% (w/v), about 0.001% (w/v) to about 0.05% (w/v), about 0.001% (w/v) to about 0.04% (w/v), about 0.001% (w/v) to about 0.03% (w/v), about 0.001% (w/v) to about 0.02% (w/v), about 0.001% (w/v) to about 0.01% (w/v), about 0.001% (w/v) to about 0.005% (w/v), about 0.001% (w/v) to about 0.002% (w/v), about 0.002% (w/v) to about 0.1% (w/v), about 0.002% (w/v) to about 0.09% (w/v), about 0.002% (w/v) to about 0.08% (w/v), about 0.002% (w/v) to about 0.07% (w/v), about 0.002% (w/v) to about 0.06% (w/v), about 0.002% (w/v) to about 0.05% (w/v), about 0.002% (w/v) to about 0.04% (w/v), about 0.002% (w/v) to about 0.03% (w/v), about 0.002% (w/v) to about 0.02% (w/v), about 0.002% (w/v) to about 0.01% (w/v), about 0.002% (w/v) to about 0.005% (w/v), about 0.005% (w/v) to about 0.1% (w/v), about 0.005% (w/v) to about 0.09% (w/v), about 0.005% (w/v) to about 0.08% (w/v), about 0.005% (w/v) to about 0.07% (w/v), about 0.005% (w/v) to about 0.06% (w/v), about 0.005% (w/v) to about 0.05% (w/v), about 0.005% (w/v) to about 0.04% (w/v), about 0.005% (w/v) to about 0.03% (w/v), about 0.005% (w/v) to about 0.02% (w/v), about 0.005% (w/v) to about 0.01% (w/v), about 0.01% (w/v) to about 0.1% (w/v), about 0.01% (w/v) to about 0.09% (w/v), about 0.01% (w/v) to about 0.08% (w/v), about 0.01% (w/v) to about 0.07% (w/v), about 0.01% (w/v) to about 0.06% (w/v), about 0.01% (w/v) to about 0.05% (w/v), about 0.01% (w/v) to about 0.04% (w/v), about 0.01% (w/v) to about 0.03% (w/v), about 0.01% (w/v) to about 0.02% (w/v), about 0.02% (w/v) to about 0.1% (w/v), about 0.02% (w/v) to about 0.09% (w/v), about 0.02% (w/v) to about 0.08% (w/v), about 0.02% (w/v) to about 0.07% (w/v), about 0.02% (w/v) to about 0.06% (w/v), about 0.02% (w/v) to about 0.05% (w/v), about 0.02% (w/v) to about 0.04% (w/v), about 0.02% (w/v) to about 0.03% (w/v), about 0.03% (w/v) to about 0.1% (w/v), about 0.03% (w/v) to about 0.09% (w/v), about 0.03% (w/v) to about 0.08% (w/v), about 0.03% (w/v) to about 0.07% (w/v), about 0.03% (w/v) to about 0.06% (w/v), about 0.03% (w/v) to about 0.05% (w/v), about 0.03% (w/v) to about 0.04% (w/v), about 0.04% (w/v) to about 0.1% (w/v), about 0.04% (w/v) to about 0.09% (w/v), about 0.04% (w/v) to about 0.08% (w/v), about 0.04% (w/v) to about 0.07% (w/v), about 0.04% (w/v) to about 0.06% (w/v), about 0.04% (w/v) to about 0.05% (w/v), about 0.05% (w/v) to about 0.1% (w/v), about 0.05% (w/v) to about 0.09% (w/v), about 0.05% (w/v) to about 0.08% (w/v), about 0.05% (w/v) to about 0.07% (w/v), about 0.05% (w/v) to about 0.06% (w/v), about 0.06% (w/v) to about 0.1% (w/v), about 0.06% (w/v) to about 0.09% (w/v), about 0.06% (w/v) to about 0.08% (w/v), about 0.06% (w/v) to about 0.07% (w/v), about 0.07% (w/v) to about 0.1% (w/v), about 0.07% (w/v) to about 0.09% (w/v), about 0.07% (w/v) to about 0.08% (w/v), about 0.08% (w/v) to about 0.1% (w/v), about 0.08% (w/v) to about 0.09% (w/v) or about 0.09% (w/v) to about 0.1% (w/v).

69. The method according to any one of claims 56 to 68, wherein the concentration of the chromogenic substrate is from about 0.005% (w/v) to about 0.02% (w/v).

70. The method according to any one of claims 56 to 69, wherein the concentration of the chromogenic substrate is about 0.01% (w/v).

71. The method according to any one of claims 56 to 70, wherein the method further comprises addition of hydrogen peroxide (H₂O₂).

72. The method according to claim 71, wherein the concentration of hydrogen peroxide is from about 0.001% (w/v) to about 0.1% (w/v), for example about 0.001% (w/v) to about 0.09% (w/v), about 0.001% (w/v) to about 0.08% (w/v), about 0.001% (w/v) to about 0.07% (w/v), about 0.001% (w/v) to about 0.06% (w/v), about 0.001% (w/v) to about 0.05% (w/v), about 0.001% (w/v) to about 0.04% (w/v), about 0.001% (w/v) to about 0.03% (w/v), about 0.001% (w/v) to about 0.02% (w/v), about 0.001% (w/v) to about 0.01% (w/v), about 0.001% (w/v) to about 0.005% (w/v), about 0.001% (w/v) to about 0.002% (w/v), about 0.002% (w/v) to about 0.1% (w/v), about 0.002% (w/v) to about 0.09% (w/v), about 0.002% (w/v) to about 0.08% (w/v), about 0.002% (w/v) to about 0.07% (w/v), about 0.002% (w/v) to about 0.06% (w/v), about 0.002% (w/v) to about 0.05% (w/v), about 0.002% (w/v) to about 0.04% (w/v), about 0.002% (w/v) to about 0.03% (w/v), about 0.002% (w/v) to about 0.02% (w/v), about 0.002% (w/v) to about 0.01% (w/v), about 0.002% (w/v) to about 0.005% (w/v), about 0.005% (w/v) to about 0.1% (w/v), about 0.005% (w/v) to about 0.09% (w/v), about 0.005% (w/v) to about 0.08% (w/v), about 0.005% (w/v) to about 0.07% (w/v), about 0.005% (w/v) to about 0.06% (w/v), about 0.005% (w/v) to about 0.05% (w/v), about 0.005% (w/v) to about 0.04% (w/v), about 0.005% (w/v) to about 0.03% (w/v), about 0.005% (w/v) to about 0.02% (w/v), about 0.005% (w/v) to about 0.01% (w/v), about 0.01% (w/v) to about 0.1% (w/v), about 0.01% (w/v) to about 0.09% (w/v), about 0.01% (w/v) to about 0.08% (w/v), about 0.01% (w/v) to about 0.07% (w/v), about 0.01% (w/v) to about 0.06% (w/v), about 0.01% (w/v) to about 0.05% (w/v), about 0.01% (w/v) to about 0.04% (w/v), about 0.01% (w/v) to about 0.03% (w/v), about 0.01% (w/v) to about 0.02% (w/v), about 0.02% (w/v) to about 0.1% (w/v), about 0.02% (w/v) to about 0.09% (w/v), about 0.02% (w/v) to about 0.08% (w/v), about 0.02% (w/v) to about 0.07% (w/v), about 0.02% (w/v) to about 0.06% (w/v), about 0.02% (w/v) to about 0.05% (w/v), about 0.02% (w/v) to about 0.04% (w/v), about 0.02% (w/v) to about 0.03% (w/v), about 0.03% (w/v) to about 0.1% (w/v), about 0.03% (w/v) to about 0.09% (w/v), about 0.03% (w/v) to about 0.08% (w/v), about 0.03% (w/v) to about 0.07% (w/v), about 0.03% (w/v) to about 0.06% (w/v), about 0.03% (w/v) to about 0.05% (w/v), about 0.03% (w/v) to about 0.04% (w/v), about 0.04% (w/v) to about 0.1% (w/v), about 0.04% (w/v) to about 0.09% (w/v), about 0.04% (w/v) to about 0.08% (w/v), about 0.04% (w/v) to about 0.07% (w/v), about 0.04% (w/v) to about 0.06% (w/v), about 0.04% (w/v) to about 0.05% (w/v), about 0.05% (w/v) to about 0.1% (w/v), about 0.05% (w/v) to about 0.09% (w/v), about 0.05% (w/v) to about 0.08% (w/v), about 0.05% (w/v) to about 0.07% (w/v), about 0.05% (w/v) to about 0.06% (w/v), about 0.06% (w/v) to about 0.1% (w/v), about 0.06% (w/v) to about 0.09% (w/v), about 0.06% (w/v) to about 0.08% (w/v), about 0.06% (w/v) to about 0.07% (w/v), about 0.07% (w/v) to about 0.1% (w/v), about 0.07% (w/v) to about 0.09% (w/v), about 0.07% (w/v) to about 0.08% (w/v), about 0.08% (w/v) to about 0.1% (w/v), about 0.08% (w/v) to about 0.09% (w/v) or about 0.09% (w/v) to about 0.1% (w/v).

73. The method according to any one of claims 71 to 72, wherein the concentration of the hydrogen peroxide is from about 0.01% (w/v) to about 0.03% (w/v).

74. The method according to any one of claims 71 to 73, wherein the concentration of hydrogen peroxide is about 0.02% (w/v).

75. Use of a nucleic acid detection system of any one of claims 1 to 43 in detection of a nucleic acid of interest.

76. The use according to claim 75 wherein the detection of a nucleic acid of interest is in a biological sample derived from a subject.

77. A kit comprising:
a nucleic acid detection system according to any one of claims 1 to 43; and
a point-of-care diagnostic test apparatus.

78. The kit according to claim 77, wherein the diagnostic test apparatus is a lateral flow immunoassay or a microfluidic chip.

79. The kit according to any one of claims 77 or 78 further comprising instructional material, optionally wherein the instructional material comprises instructions for conducting a method of detecting a nucleic acid of interest or diagnosing a disease in a patient according to any one of claims 56 to 74.

80. The kit according to any one of claims 77 to 79, further comprising a chromogenic substrate.

81. The kit according to claim 80, wherein the chromogenic substrate is selected from the list consisting of: CN/DAB (4-chloro-1-naphthol/3,3′-diaminobenzidine,
tetrahydrochloride), TMB (3,3′,5,5′-tetramethylbenzidine) PNPP (p-Nitrophenyl Phosphate) ABTS (2,2′-Azinobis [3-ethylbenzothiazoline-6-sulfonic acid]), OPD (o-phenylenediamine dihydrochloride) and ONGP (ortho-Nitrophenyl-β-galactoside).

82. The kit according to any one of claims 80 to 81, wherein the chromogenic substrate is 3,3′,5,5′-tetramethylbenzidine (TMB).

83. A diagnostic assay apparatus comprising the nucleic acid detection system of any one of claims 1 to 43.

84. The kit according to claim 77, wherein the diagnostic test apparatus is a lateral flow immunoassay.

85. The kit according to claim 84, wherein the lateral flow immunoassay comprises a nitrocellulose test strip.

86. The kit according to claim 84 or claim 85, wherein the lateral flow immunoassay comprises an anti-digoxigenin antibody, a biotin-binding protein and/or an anti-FITC antibody.

87. The kit according to claim 84 or claim 85, wherein the lateral flow immunoassay comprises an anti-FITC antibody.

88. The kit according to any one of claims 80 to 81 wherein the chromogenic substrate is 4-chloro-1-naphthol/3,3′-diaminobenzidine, tetrahydrochloride (CN/DAB).


SEQUENCE LISTING (17 sequences)

SEQ ID NO: 1 - RNA, 71 nt, synthetic construct
  source 1..71: mol_type = other RNA
  misc_feature 1..71: note = Artificial sequence
tgacgggcgagcttttggcccgggttatacctgatgctcacgtataagacgagcaaaaagcttgttggtca

SEQ ID NO: 2 - RNA, 21 nt, synthetic construct
  source 1..21: mol_type = other RNA
  misc_feature 1..21: note = miR-223
tgtcagtttgtcaaatacccc

SEQ ID NO: 3 - RNA, 117 nt, synthetic construct
  source 1..117: mol_type = other RNA
  misc_feature 1..117: note = lnc-LIPCAR
catgatcacgccctcataatcattttccttatctgcttcctagtcctgtatgcccttttcctaacactcacaacaaaactaactaatactaacatctcagacgctcaggaaatagaa

SEQ ID NO: 4 - DNA, 187 bp, synthetic construct
  source 1..187: mol_type = other DNA
  misc_feature 1..187: note = circ-AURKA
gaaattaatacgactcactataggtttacaggtaatggattctgacaaggaaattgctgagtcacgagaacacgttttggacctccaactggagctgtagccagtagcagttctctgctcatcaaactttgaaagtttctgaagttctctataaactgttccaagtggtgcatattccagaattagg

SEQ ID NO: 5 - DNA, 47 bp, synthetic construct
  source 1..47: mol_type = other DNA
  misc_feature 1..47: note = Synthetic RNA forward primer
gaaattaatacgactcactataggtgacgggcgagcttttggcccgg

SEQ ID NO: 6 - DNA, 30 bp, synthetic construct
  source 1..30: mol_type = other DNA
  misc_feature 1..30: note = Synthetic RNA reverse primer
gacctagccgtgaccaacaagctttttgct

SEQ ID NO: 7 - DNA, 56 bp, synthetic construct
  source 1..56: mol_type = other DNA
  misc_feature 1..56: note = circAURKA forward primer
gaaattaatacgactcactataggtttacaggtaatggattctgacaaggaaattg

SEQ ID NO: 8 - DNA, 30 bp, synthetic construct
  source 1..30: mol_type = other DNA
  misc_feature 1..30: note = circAURKA reverse primer
cacttggaacagtttatagagaacttcaga

SEQ ID NO: 9 - DNA, 88 bp, synthetic construct
  source 1..88: mol_type = other DNA
  misc_feature 1..88: note = Synthetic RNA target sequence
gttatacctgatgctcacgtataagacggttttagtccccttcgtttttggggtagtctaaatccctatagtgagtcgtattaatttc

SEQ ID NO: 10 - DNA, 81 bp, synthetic construct
  source 1..81: mol_type = other DNA
  misc_feature 1..81: note = miR-223 target sequence
tgtcagtttgtcaaataccccgttttagtccccttcgtttttggggtagtctaaatccctatagtgagtcgtattaatttc

SEQ ID NO: 11 - DNA, 87 bp, synthetic construct
  source 1..87: mol_type = other DNA
  misc_feature 1..87: note = lnc-LIPCAR target sequence
ctgtatgcccttttcctaacactcacaagttttagtccccttcgtttttggggtagtctaaatccctatagtgagtcgtattaattt

SEQ ID NO: 12 - DNA, 88 bp, synthetic construct
  source 1..88: mol_type = other DNA
  misc_feature 1..88: note = circ-AURKA target sequence
cagttctctgctcatcaaactttgaaaggttttagtccccttcgtttttggggtagtctaaatccctatagtgagtcgtattaatttc

SEQ ID NO: 13 - DNA, 81 bp, synthetic construct
  source 1..81: mol_type = other DNA
  misc_feature 1..81: note = miR-143 target sequence
tgagatgaagcactgtagctcgttttagtccccttcgtttttggggtagtctaaatccctatagtgagtcgtattaatttc

SEQ ID NO: 14 - DNA, 215 bp, synthetic construct
  source 1..215: mol_type = other DNA
  misc_feature 1..215: note = 193 base pair synthetic RNA coding sequence
gaaattaatacgactcactataggatcaaagcacttacatgagggggcaaagtcagagacagctgaggagctgaagaaggtggctcaggagctggaggagaagctaaacattctcaacaataagattctgcaggcggaccaagaactgtgaccacagggcagggcagccaccaggagagatatgcctggcaggggccaggacaaaatgcaaactt

SEQ ID NO: 15 - DNA, 56 bp, synthetic construct
  source 1..56: mol_type = other DNA
  misc_feature 1..56: note = LIPCAR forward primer
gaaattaatacgactcactataggtcataatcattttccttatctgcttcctagtc

SEQ ID NO: 16 - DNA, 32 bp, synthetic construct
  source 1..32: mol_type = other DNA
  misc_feature 1..32: note = LIPCAR reverse primer sequence
tatttcctgagcgtctgagatgttagtattag

SEQ ID NO: 17 - DNA, 74 bp, synthetic construct
  misc_feature 1..74: note = 193 base pair synthetic RNA
  source 1..74: mol_type = other DNA
agctaaacattctcaacaatgttttagtccccttcatttttggggtggtccctatagtgagtcgtattaatttc